(19) United States
(12) Patent Application Publication
(10) Pub. No.: US 2025/0353809 A1
(43) Pub. Date: Nov. 20, 2025

(54) NITROGEN-CONTAINING CHAIN COMPOUND, PREPARATION METHOD, COMPOSITION CONTAINING SAID COMPOUND, AND USE THEREOF

(71) Applicant: SHANGHAI RNACURE BIOPHARMA CO., LTD., Shanghai (CN)

(72) Inventors: Jinzhong LIN, Shanghai (CN); Jing LU, Shanghai (CN); Hang YU, Shanghai (CN); Ting JIANG, Shanghai (CN); Fan ZHANG, Shanghai (CN); Boyang ZHANG, Shanghai (CN); Junxiang GUO, Shanghai (CN); Lin CHEN, Shanghai (CN); Bing WANG, Shanghai (CN)

(21) Appl. No.: 18/871,852

(22) PCT Filed: Jun. 6, 2023
(86) PCT No.: PCT/CN2023/098621

§ 371 Date: Dec. 5, 2024

(30) Foreign Application Priority Data
Jun. 6, 2022 (CN) 202210652431.0
Mar. 17, 2023 (CN) 202310272482.5
May 25, 2023 (CN) 202310603492.2

(51) Int. Cl. C07C 229/16; A61K 9/51; A61K 45/06; A61P 31/04; A61P 31/12

(52) U.S. Cl. CPC C07C 229/16; A61K 9/5123; A61K 45/06; A61P 31/04; A61P 31/12

(57) ABSTRACT

Disclosed are a nitrogen-containing chain compound, a preparation method, a composition containing said compound, and a use thereof. Provided is a nitrogen-containing chain compound represented by formula (I) or a pharmaceutically acceptable salt thereof. The nitrogen-containing chain compound represented by formula (I) may be used for preparing a lipid carrier. The prepared lipid carrier can encapsulate a nucleic acid drug, and may be used for delivering a nucleic acid prophylactic agent and/or therapeutic agent to mammalian cells and organs and producing an effect.

The present application claims priority to Chinese patent application 2022106524310, filed on Jun. 6, 2022, priority to Chinese patent application 2023102724825, filed on Mar. 17, 2023, and priority to Chinese patent application 2023106034922, filed on May 25, 2023, and the entire contents of the Chinese patent applications above are cited in the present application.

### TECHNICAL FIELD

The present disclosure relates to a nitrogen-containing chain compound, a preparation method, a composition containing said compound, and a use thereof.

### BACKGROUND

Nucleic acid drugs are an important direction in current basic and applied research. The nucleic acid drugs may be used to prevent and/or treat viral and bacterial infectious diseases, tumors, metabolic diseases, etc. The nucleic acid drugs have lower production costs and shorter cycle, which is conducive to the rapid development of personalized drugs. However, nucleic acids are electronegative large molecules that hardly penetrate through cell membranes, and meanwhile have poor stability. By developing various nucleic acid packaging and delivery systems, instability of the nucleic acid drugs may be overcome to some extent, and their delivery efficiency may be improved.

Lipid nanoparticles have been proven to be used as carriers for delivering bioactive substances (such as small molecule drugs, proteins, and nucleic acids) into cells and/or intracellular compartments. The optimization of a nucleic acid drug delivery system by designing and optimizing the type and amount of each component in the lipid nanoparticles is of great significance for improving the prevention and treatment efficacy of the nucleic acid drugs, especially a lipid compound that can be used to deliver an RNA prophylactic agent and/or therapeutic agent, and related methods and compositions.

### CONTENT OF THE PRESENT INVENTION

The present invention aims to provide a novel ionizable lipid compound for delivering a nucleic acid drug, which can increase types of ionizable lipid compounds and the selection of delivery carriers for a nucleic acid prophylactic agent and/or therapeutic agent. To solve the above technical problem, the present invention provides a nitrogen-containing chain compound, a preparation method, a composition containing said compound, and a use thereof. The composition of the present invention may be used for efficiently delivering a nucleic acid drug. The technical solutions of the present invention are as follows:

The present invention provides a nitrogen-containing chain compound represented by a formula (I) or a pharmaceutically acceptable salt thereof,

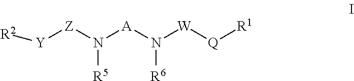

wherein, Z and W are independently C₃-C₁₀ alkylene;
Y and Q are independently;

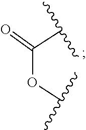 [SMILES: *OC(*)=O]

A is C₂-C₆ alkylene,

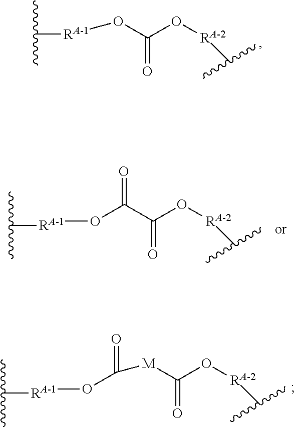

R^{A-1} and R^{A-2} are each independently C₂-C₆ alkylene;
M is C₁-C₆ alkylene;
R¹ and R² are independently C₆-C₂₀ alkyl;
R⁵ is C₂-C₁₀ alkyl that is unsubstituted or substituted with 1, 2, or 3 R⁵⁻¹;
each R⁵⁻¹ is independently hydroxyl or

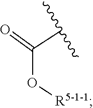

each R⁵⁻¹⁻¹ is independently C₆-C₂₀ alkyl;
R⁶ is C₂-C₁₀ alkyl that is unsubstituted or substituted with 1, 2, or 3 R⁶⁻¹;
each R⁶⁻¹ is independently hydroxyl or

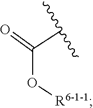

and
each R⁶⁻¹⁻¹ is independently C₆-C₂₀ alkyl.

The present invention provides a nitrogen-containing chain compound represented by a formula (I) or a pharmaceutically acceptable salt thereof,

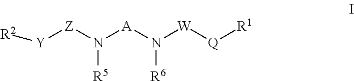

wherein, Z and W are independently C₃-C₁₀ alkylene;
Y and Q are independently

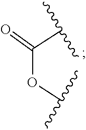 [SMILES: *OC(*)=O]

A is C₂-C₆ alkylene,

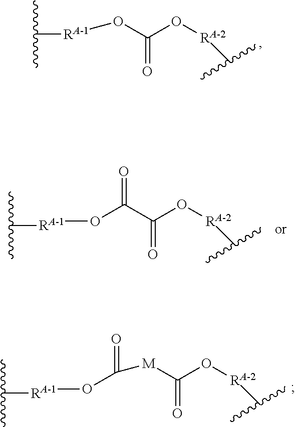

R^{A-1} and R^{A-2} are each independently C₂-C₆ alkylene;
M is C₁-C₆ alkylene;
R¹ and R² are independently C₆-C₂₀ alkyl;
R⁵ is C₂-C₁₀ alkyl that is unsubstituted or substituted with 1, 2, or 3 R⁶⁻¹;
each R⁵⁻¹ is independently hydroxyl or

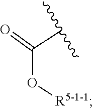

R⁵⁻¹⁻¹ is independently C₆-C₂₀ alkyl;
R⁶ is C₂-C₁₀ alkyl that is unsubstituted or substituted with 1, 2, or 3 R⁶⁻¹;
each R⁶⁻¹ is independently hydroxyl or

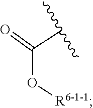

and
R⁶⁻¹⁻¹ is independently C₆-C₂₀ alkyl.

The present invention provides a nitrogen-containing chain compound represented by a formula (I) or a pharmaceutically acceptable salt thereof,

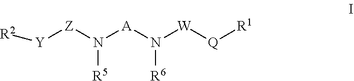

wherein, Z and W are independently C₄-C₁₀ alkylene;
Y and Q are independently

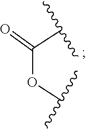 [SMILES: *OC(*)=O]

A is C₂-C₆ alkylene,

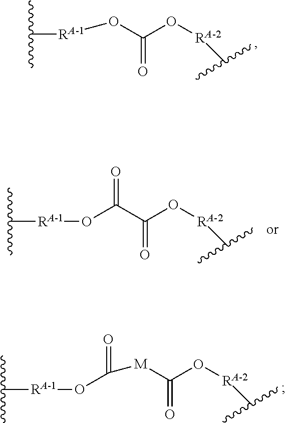

R^{A-1} and R^{A-2} are each independently C₂-C₆ alkylene;
M is C₁-C₆ alkylene;
R¹ and R² are independently C₆-C₂₀ alkyl;
R⁵ is C₂-C₁₀ alkyl that is unsubstituted or substituted with 1, 2, or 3 R⁵⁻¹;
each R⁵⁻¹ is independently hydroxyl or

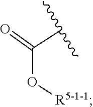

R⁵⁻¹⁻¹ is independently C₆-C₂₀ alkyl;
R⁶ is C₂-C₁₀ alkyl that is unsubstituted or substituted with 1, 2, or 3 R⁶⁻¹;
each R⁶⁻¹ is independently hydroxyl or

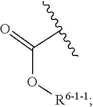

and
R⁶⁻¹⁻¹ is independently C₆-C₂₀ alkyl.

In a certain preferred solution, in the nitrogen-containing chain compound represented by the formula (I) or the pharmaceutically acceptable salt thereof, definitions of certain groups may be described below, while definitions of other groups may be described as any other solution (hereinafter referred to as “a certain preferred solution”): in Z, C₄-C₁₀ alkylene may be C₅-C₈ alkylene, preferably straight-chain alkane, such as

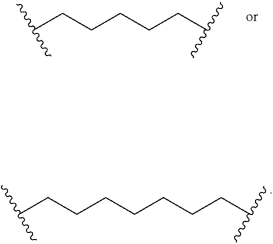 [SMILES: *CCCCC* ; *CCCCCCC*]

In a certain preferred solution, in Z, C₃-C₁₀ alkylene may be C₃-C₈ alkylene, preferably straight-chain alkane such as

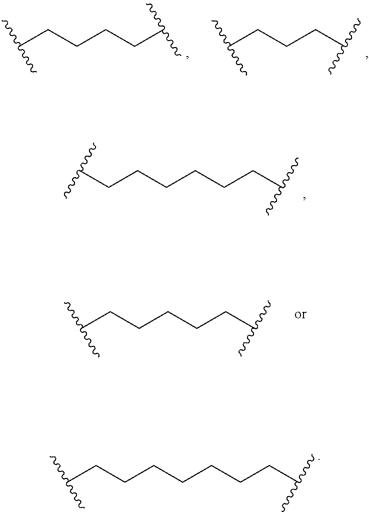 [SMILES: *CCCC* ; *CCC* ; *CCCCCC* ; *CCCCC* ; *CCCCCCC*]

In a certain preferred solution, in W, C₄-C₁₀ alkylene may be C₄-C₁₀ alkylene, and may further be C₅-C₈ alkylene, preferably straight-chain alkane, such as

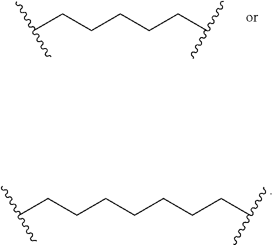 [SMILES: *CCCCC* ; *CCCCCCC*]

In a certain preferred solution, in W, C₃-C₁₀ alkylene may be C₃-C₈ alkylene, preferably straight-chain alkane, such as

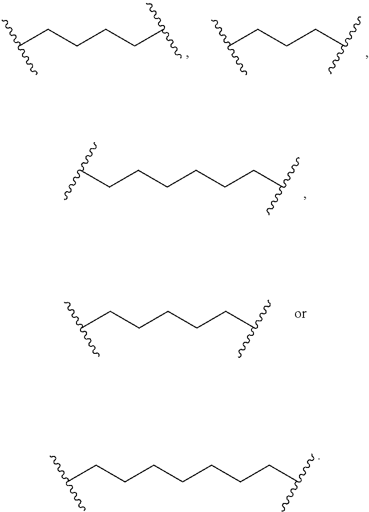 [SMILES: *CCCC* ; *CCC* ; *CCCCCC* ; *CCCCC* ; *CCCCCCC*]

In a certain preferred solution, in A, C₂-C₆ alkylene may be

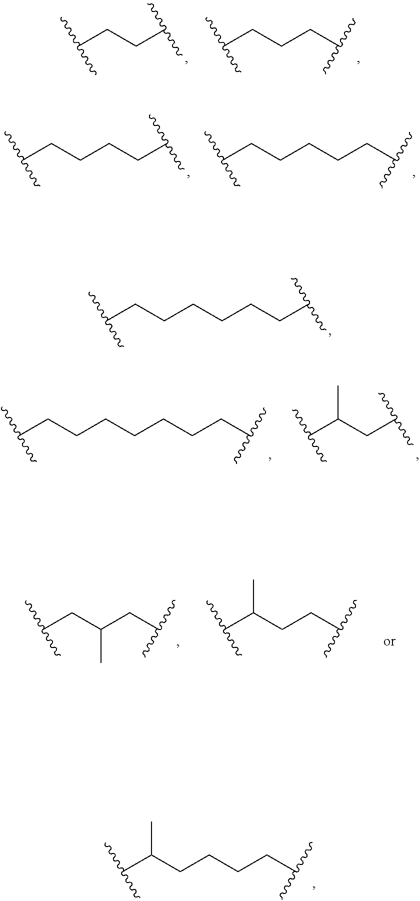

such as

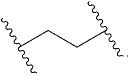 [SMILES: *CC*]

In a certain preferred solution, in A, C₂-C₆ alkylene may be

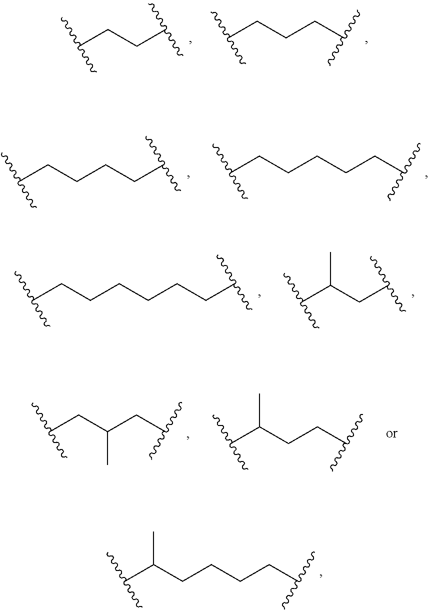

such as

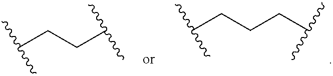

In a certain preferred solution, in R^{A-1}, C₂-C₆ alkylene may be

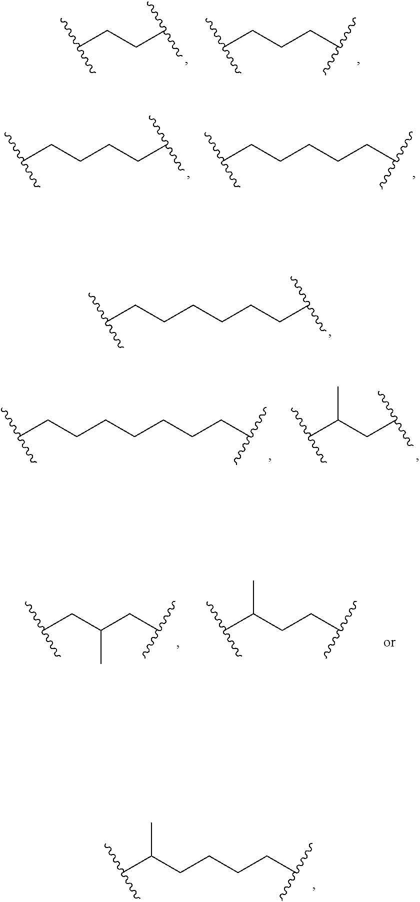

such as

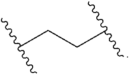 [SMILES: *CC*]

In a certain preferred solution, in R^{A-2}, C₂-C₆ alklene may be

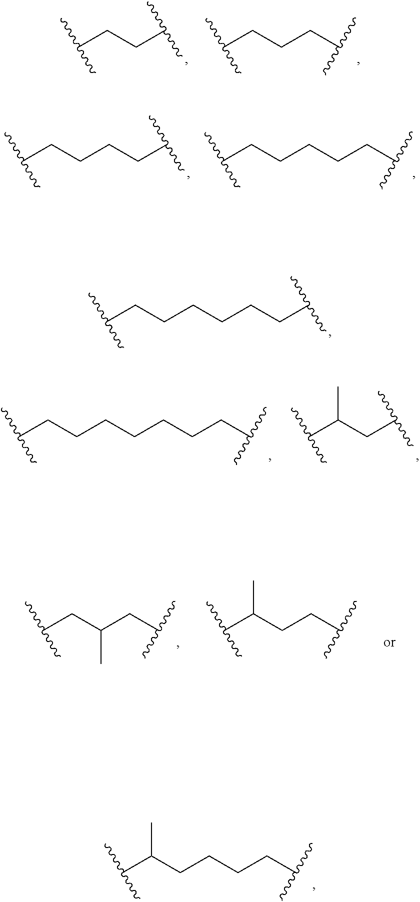

such as

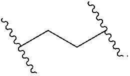 [SMILES: *CC*]

In a certain preferred solution in M, C₁-C₆ alkylene may be

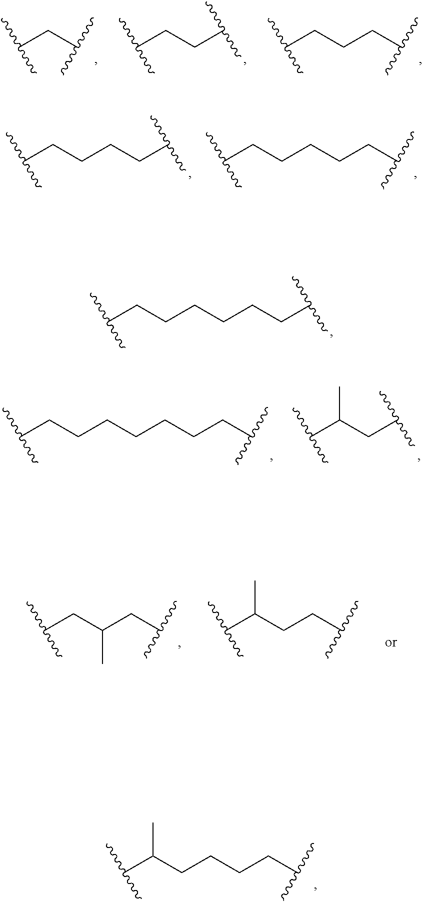

such as

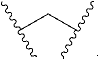 [SMILES: *C*]

In a certain preferred solution, in R¹, C₆-C₂₀ alkyl may be C₁₀-C₁₈, such as

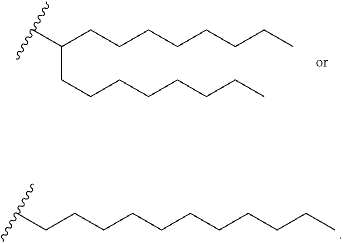 [SMILES: *C(CCCCCCCC)CCCCCCCC ; *CCCCCCCCCCC]

In a certain preferred solution, in R¹, C₆-C₂₀ alkyl may be C₁₀-C₁₉, such as

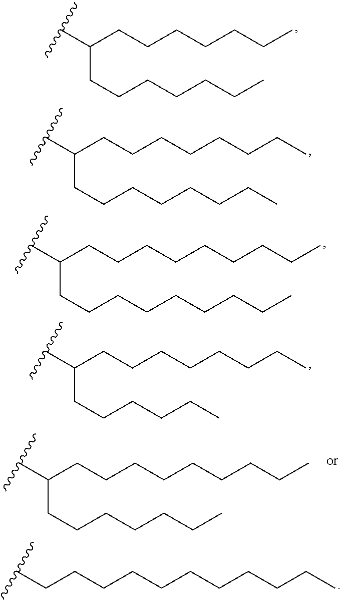

In a certain preferred solution, in R², C₆-C₂₀ alkyl may be C₁₀-C₁₈, such as

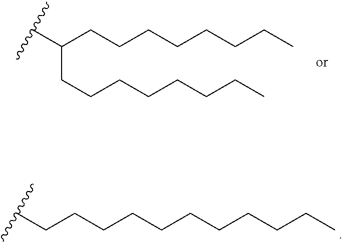 [SMILES: *C(CCCCCCCC)CCCCCCCC ; *CCCCCCCCCCC]

In a certain preferred solution, in R², C₆-C₂₀ alkyl may be C₁₀-C₁₉, such as

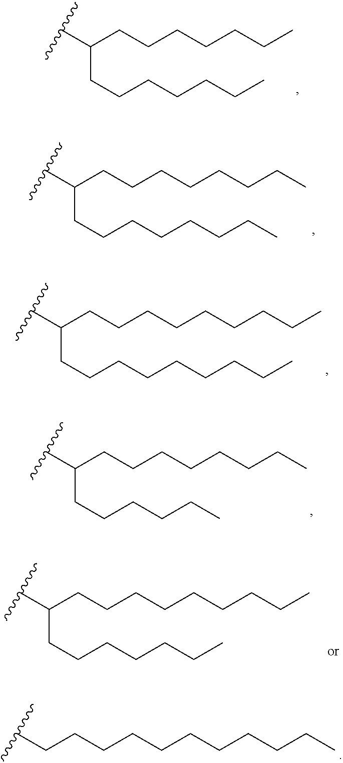 [SMILES: *C(CCCCCCC)CCCCCCC ; *C(CCCCCCCC)CCCCCCCC ; *C(CCCCCCCCC)CCCCCCCCC ; *C(CCCCCC)CCCCCCCC ; *C(CCCCCCC)CCCCCCCCC ; *CCCCCCCCCCC]

In a certain preferred solution, in R⁵, C₂-C₁₀ alkyl may be C₂-C₈ alkyl, such as

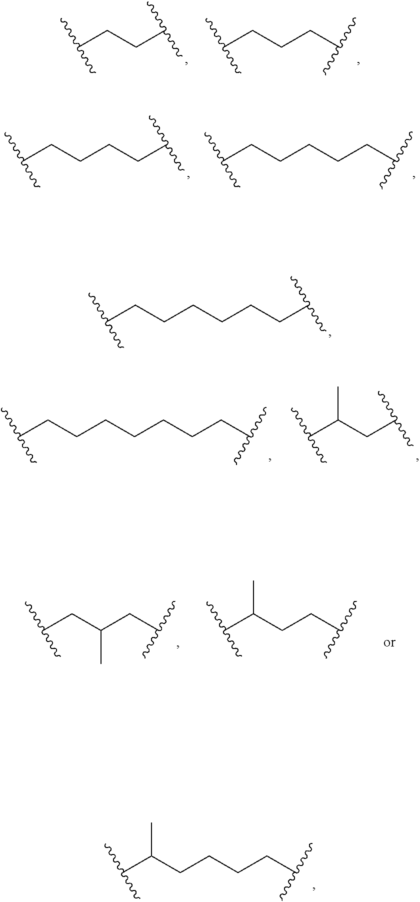

and also such as

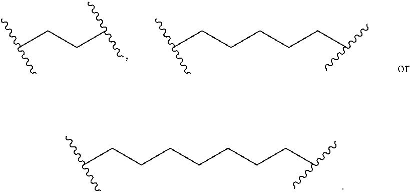

In a certain preferred solution, in R⁵⁻¹⁻¹, C₆-C₂₀ alkyl may be C₁₁-C₁₈, such as

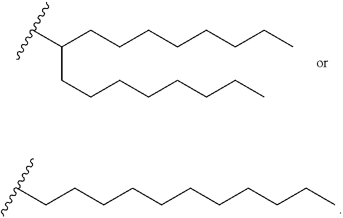 [SMILES: *C(CCCCCCCC)CCCCCCCC ; *CCCCCCCCCCC]

In a certain preferred solution, in R⁶, C₂-C₁₀ alkyl may be C₂-C₈ alkyl, such as

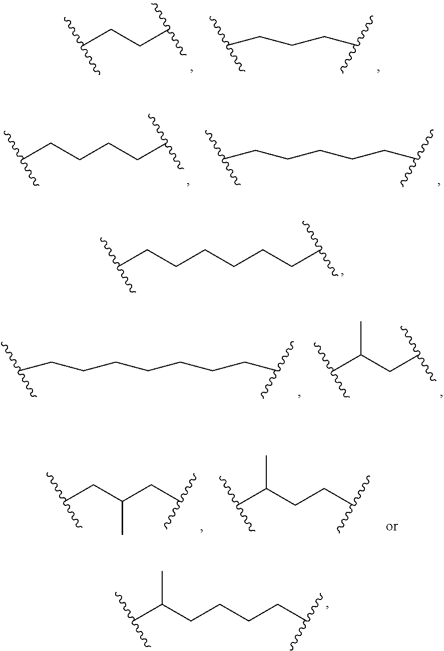

and also such as

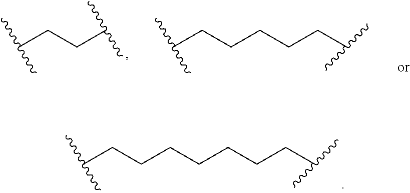

In a certain preferred solution, in R⁶⁻¹⁻¹, C₆-C₂₀ alkyl may be C₁₁-C₁₈, such as

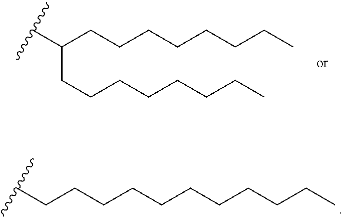 [SMILES: *C(CCCCCCCC)CCCCCCCC ; *CCCCCCCCCCC]

In a certain preferred solution, the nitrogen-containing chain compound represented by the formula (I) is a nitrogen-containing chain compound represented by a formula (I-a)

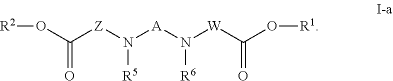

In a certain preferred solution, Y is

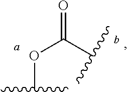

wherein a is connected to R², and b is connected to Z.

In a certain preferred solution, Q is

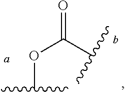

wherein a is connected to R¹, and b is connected to W.

In a certain preferred solution, Q and Y are the same.

In a certain preferred solution, Z and W are the same.

In a certain preferred solution, R¹ and R² are the same.

In a certain preferred solution, R⁵ and R⁶ are the same.

In a certain preferred solution, Z and W are independently C₅-C₈ alkylene.

In a certain preferred solution, Z and W are independently C₃-C₈ alkylene.

In a certain preferred solution, A is C₂-C₆ alkylene, or

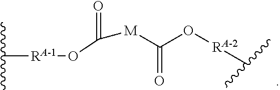

In a certain preferred solution, R^{A-1} and R^{A-2} are independently C₂-C₄ alkylene.

In a certain preferred solution, M is methylene.

In a certain preferred solution, R¹ and R² are independently C₁₀-C₁₈, such as C₁₀-C₁₂, and also such as

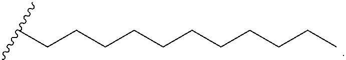 [SMILES: *CCCCCCCCCCC]

In a certain preferred solution, R¹ and R² are independently C₁₀-C₂₀ alkyl, preferably,

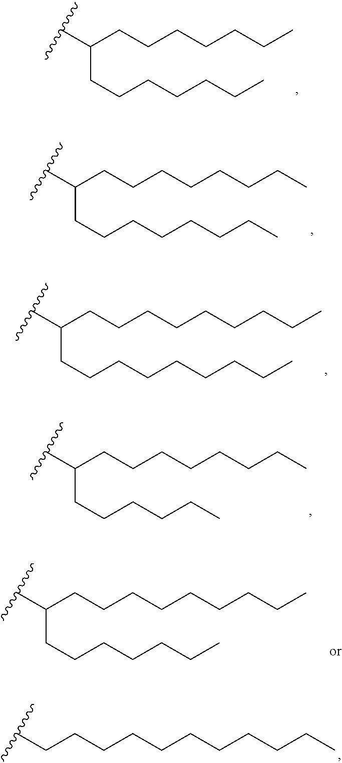 [SMILES: *C(CCCCCCC)CCCCCCC ; *C(CCCCCCCC)CCCCCCCC ; *C(CCCCCCCCC)CCCCCCCCC ; *C(CCCCCC)CCCCCCCC ; *C(CCCCCCC)CCCCCCCCC ; *CCCCCCCCCCC]

and more preferably

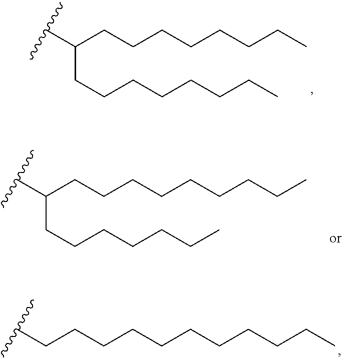 [SMILES: *C(CCCCCCCC)CCCCCCCC ; *C(CCCCCCC)CCCCCCCCC ; *CCCCCCCCCCC]

In a certain preferred solution, R⁵ is C₂-C₈ alkyl that is substituted with 1, 2, or 3 R⁵⁻¹.

In a certain preferred solution, R⁵⁻¹⁻¹ is C₁₀-C₁₈ alkyl, such as C₁₄-C₁₈ alkyl, and also such as

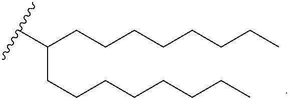 [SMILES: *C(CCCCCCCC)CCCCCCCC]

In a certain preferred solution, R⁶ is C₂-C₈ alkyl that is substituted with 1, 2, or 3 R⁶⁻¹.

In a certain preferred solution, R⁶⁻¹⁻¹ is C₁₀-C₁₈ alkyl, such as C₁₄-C₁₈ alkyl, and also such as

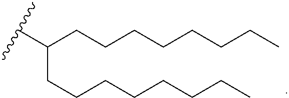 [SMILES: *C(CCCCCCCC)CCCCCCCC]

In a certain preferred solution, Y is

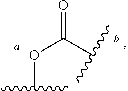

Wherein a is connected to R², and b is connected to Z;
Q is

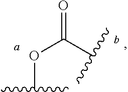

wherein a is connected to R¹, and b is connected to W;
Z and W are independently C₃-C₈ alkylene;
A is C₂-C₆ alkylene, or

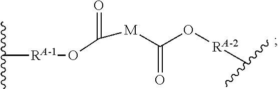

R^{A-1} and R^{A-2} are independently C₂-C₄ alkylene;
M is methylene;
R¹ and R² are independently C₁₀-C₂₀ alkyl;
R⁵ is C₂-C₈ alkyl that is substituted with 1, 2, or 3 R⁵⁻¹;
R⁵⁻¹⁻¹ is C₁₀-C₁₈ alkyl;
R⁶ is C₂-C₈ alkyl that is substituted with 1, 2, or 3 R⁶⁻¹; and
R⁶⁻¹⁻¹ is C₁₀-C₁₈ alkyl.

In a certain preferred solution, Y is

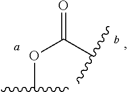

wherein a is connected to R², and b is connected to Z;
Q is

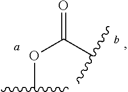

wherein a is connected to R¹, and b is connected to W;
Z and W are independently C₅-C₈ alkylene;
A is C₂-C₆ alkylene, or

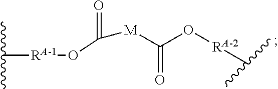

R^{A-1} and R^{A-2} are independently C₂-C₄ alkylene;
M is methylene;
R¹ and R² are independently C₁₀-C₁₈;
R⁵ is C₂-C₈ alkyl that is substituted with 1, 2, or 3 R⁵⁻¹;
R⁵⁻¹⁻¹ is C₁₀-C₁₈ alkyl;
R⁶ is C₂-C₈ alkyl that is substituted with 1, 2, or 3 R⁶⁻¹; and
R⁶⁻¹⁻¹ is C₁₀-C₁₈ alkyl.

In a certain preferred solution, Q and Y are the same;
Z and W are the same;
R¹ and R² are the same;
R⁵ and R⁶ are the same;
Z and W are independently C₅-C₈ alkylene;
A is C₂-C₆ alkylene, or

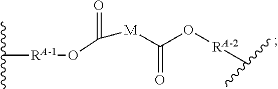

R^{A-1} and R^{A-2} are independently C₂-C₄ alkylene;
M is methylene;
R¹ and R² are independently

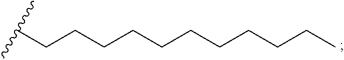 [SMILES: *CCCCCCCCCCC]

R⁵ is C₂-C₈ alkyl that is substituted with 1, 2, or 3 R⁵⁻¹;
R⁵⁻¹⁻¹ is C₁₄-C₁₈ alkyl;
R⁶ is C₂-C₈ alkyl that is substituted with 1, 2, or 3 R⁶⁻¹; and
R⁶⁻¹⁻¹ is C₁₄-C₁₈ alkyl.

In a certain preferred solution, the nitrogen-containing chain compound represented by the formula (I) is a bilaterally symmetrical compound.

In a certain preferred solution, Z may be

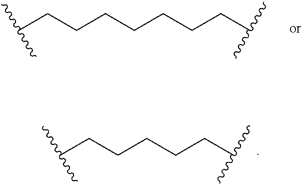 [SMILES: *CCCCCCC* ; *CCCCC*]

In a certain preferred solution, Z may be

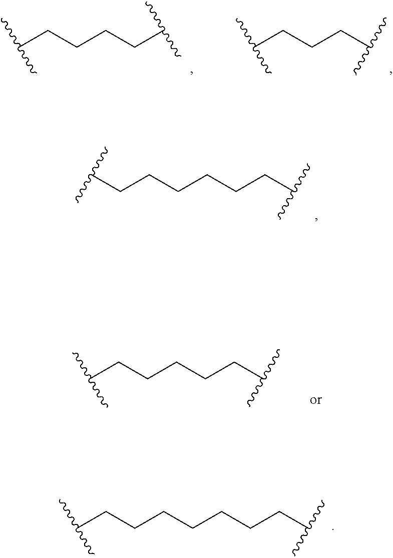 [SMILES: *CCCC* ; *CCC* ; *CCCCCC* ; *CCCCC* ; *CCCCCCC*]

In a certain preferred solution, W may be

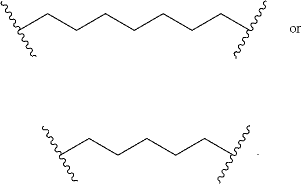 [SMILES: *CCCCCCC* ; *CCCCC*]

In a certain preferred solution, W may be

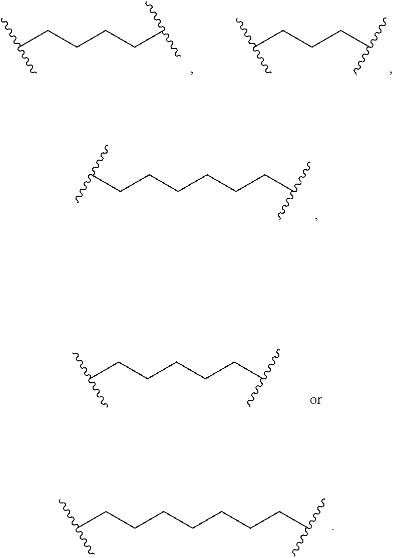 [SMILES: *CCCC* ; *CCC* ; *CCCCCC* ; *CCCCC* ; *CCCCCCC*]

In a certain preferred solution, R¹ may be

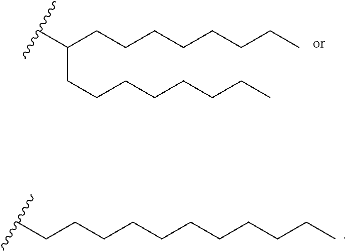 [SMILES: *C(CCCCCCCC)CCCCCCCC ; *CCCCCCCCCCC]

In a certain preferred solution, R¹ may be

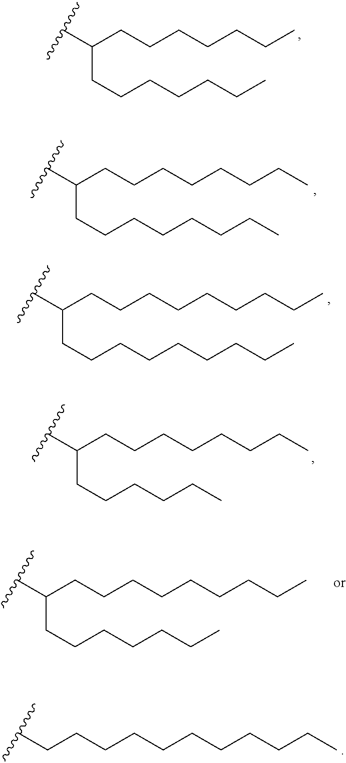 [SMILES: *C(CCCCCCC)CCCCCCC ; *C(CCCCCCCC)CCCCCCCC ; *C(CCCCCCCCC)CCCCCCCCC ; *C(CCCCCC)CCCCCCCC ; *C(CCCCCCC)CCCCCCCCC ; *CCCCCCCCCCC]

In a certain preferred solution, R² may be

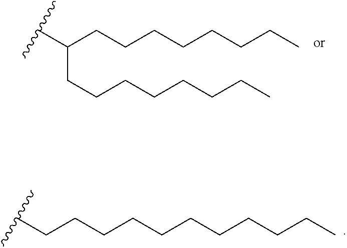 [SMILES: *C(CCCCCCCC)CCCCCCCC ; *CCCCCCCCCCC]

In a certain preferred solution, R² may be

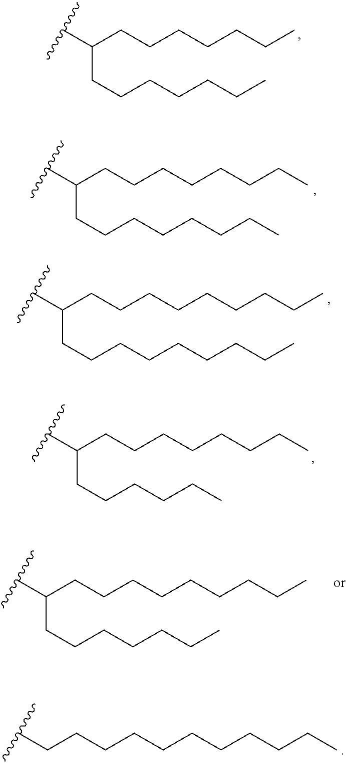 [SMILES: *C(CCCCCCC)CCCCCCC ; *C(CCCCCCCC)CCCCCCCC ; *C(CCCCCCCCC)CCCCCCCCC ; *C(CCCCCC)CCCCCCCC ; *C(CCCCCCC)CCCCCCCCC ; *CCCCCCCCCCC]

In a certain preferred solution, R⁵ may be

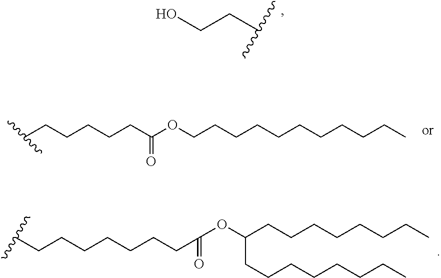

In a certain preferred solution, R⁶ may be

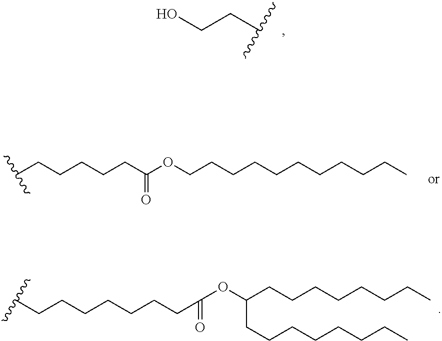

In a certain preferred solution, A may be

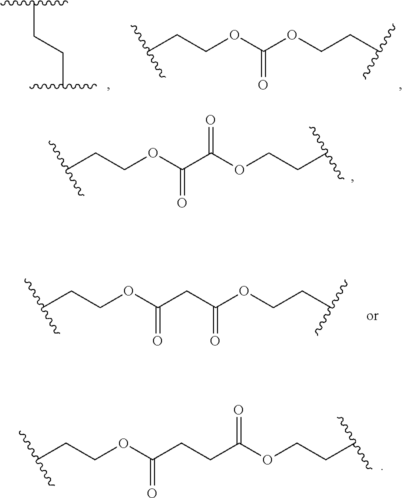

In a certain preferred solution, A may be

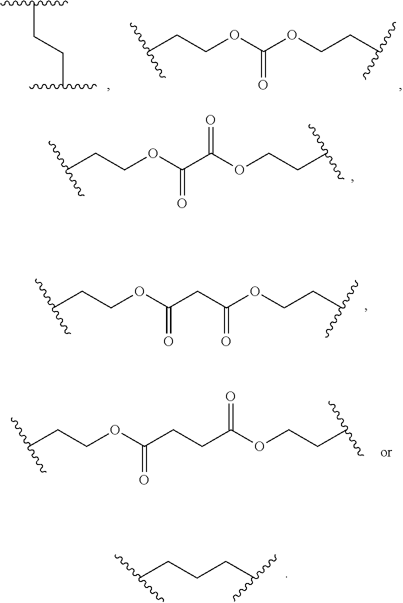

In a certain preferred solution, the nitrogen-containing chain compound represented by the formula (I) is any of the following compounds:

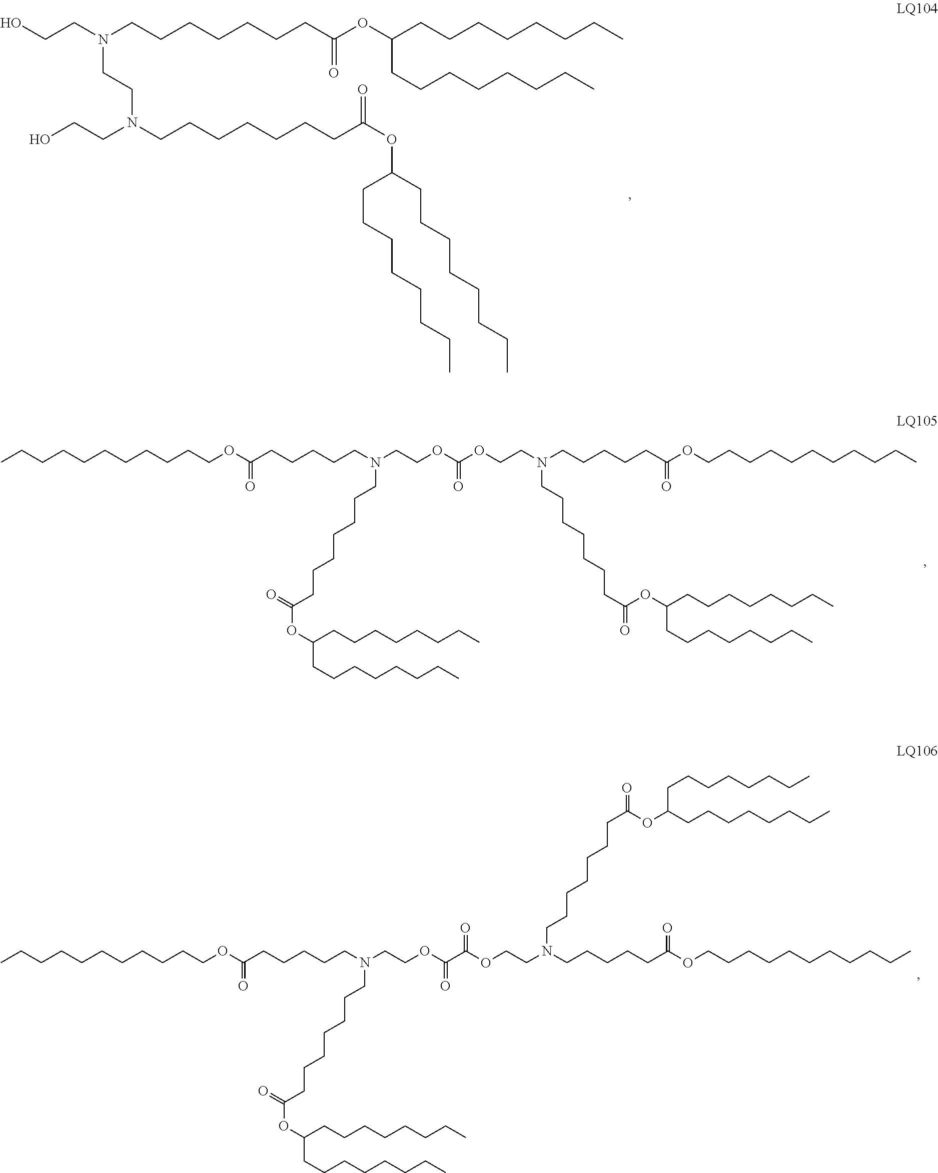

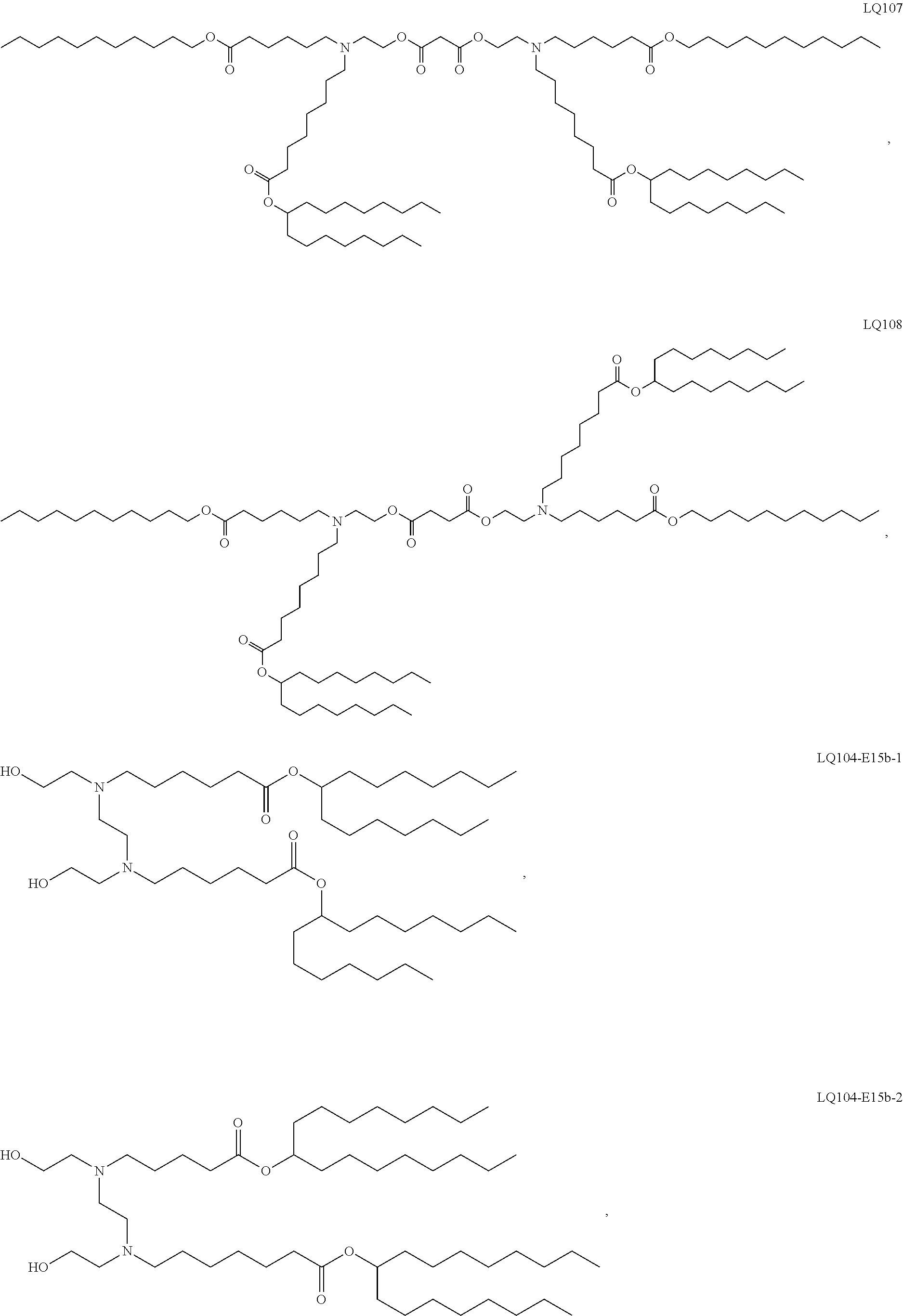

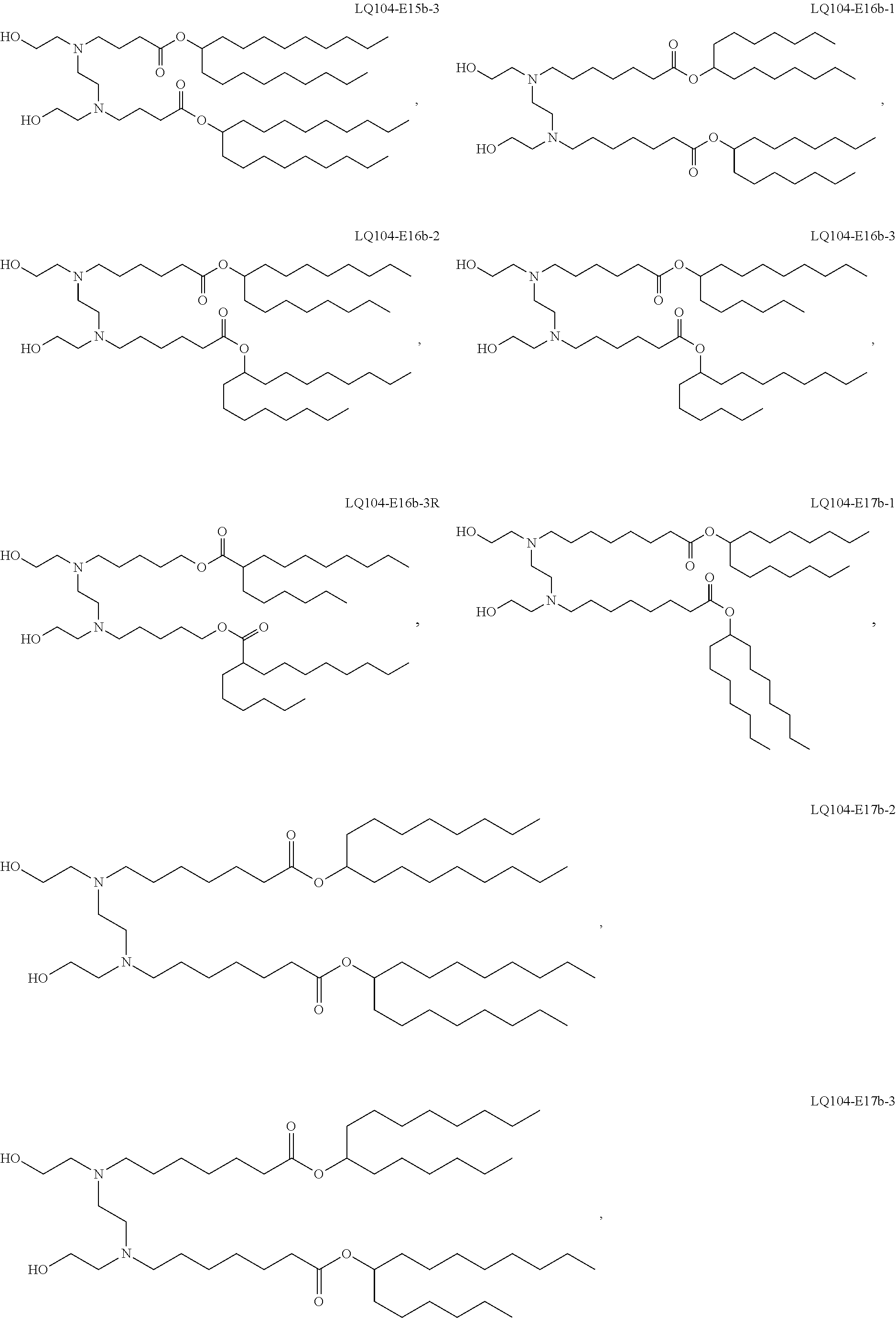

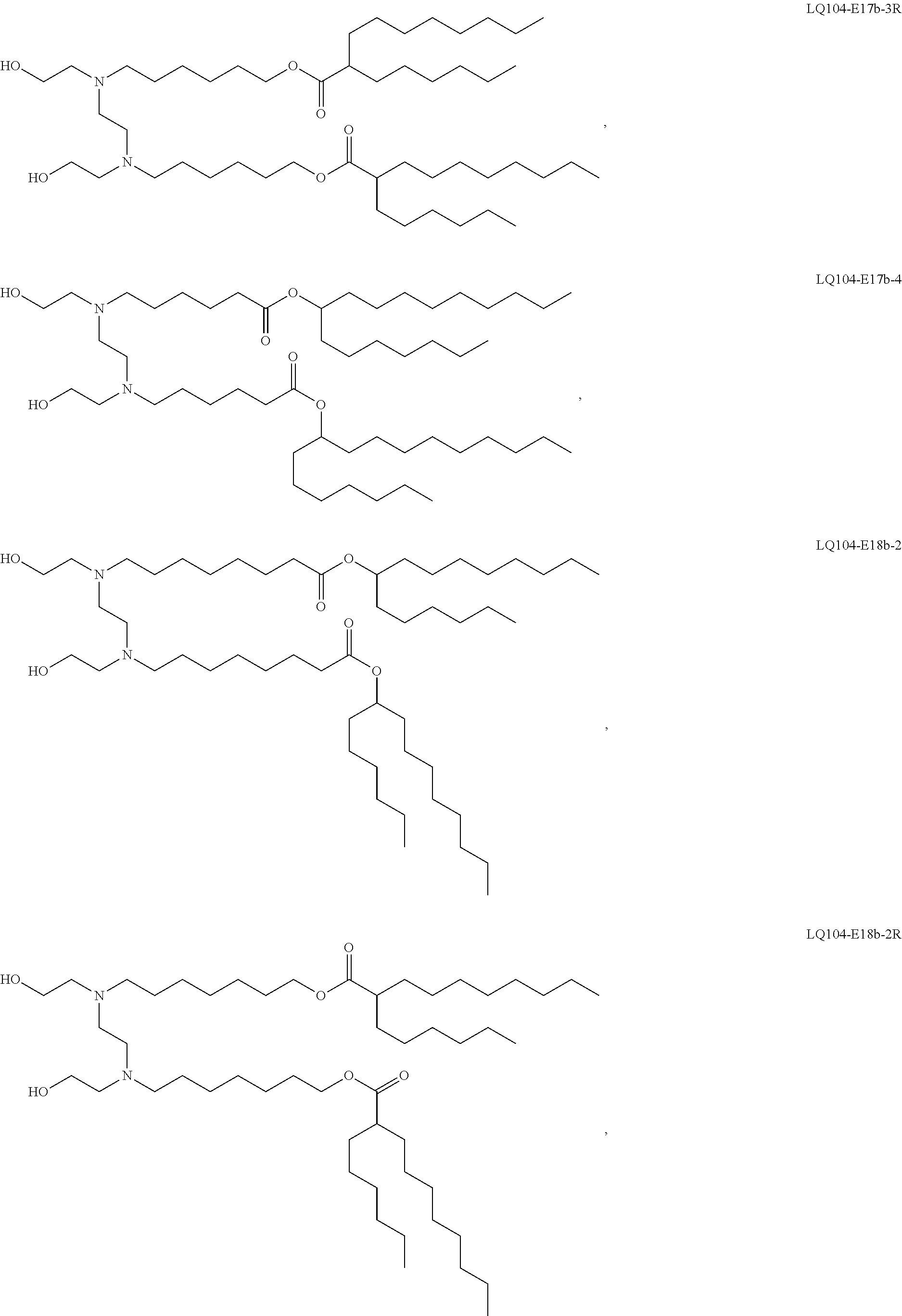 [SMILES: CCCCCCCCC(CCCCCC)C(=O)OCCCCCCN(CCO)CCN(CCO)CCCCCCOC(=O)C(CCCCCC)CCCCCCCC ; CCCCCCCCCC(CCCCCCC)OC(=O)CCCCCN(CCO)CCN(CCO)CCCCCC(=O)OC(CCCCCCC)CCCCCCCCC ; CCCCCCCCC(CCCCCC)OC(=O)CCCCCCCN(CCO)CCN(CCO)CCCCCCCC(=O)OC(CCCCCC)CCCCCCCC ; CCCCCCCCC(CCCCCC)C(=O)OCCCCCCCN(CCO)CCN(CCO)CCCCCCCOC(=O)C(CCCCCC)CCCCCCCC]

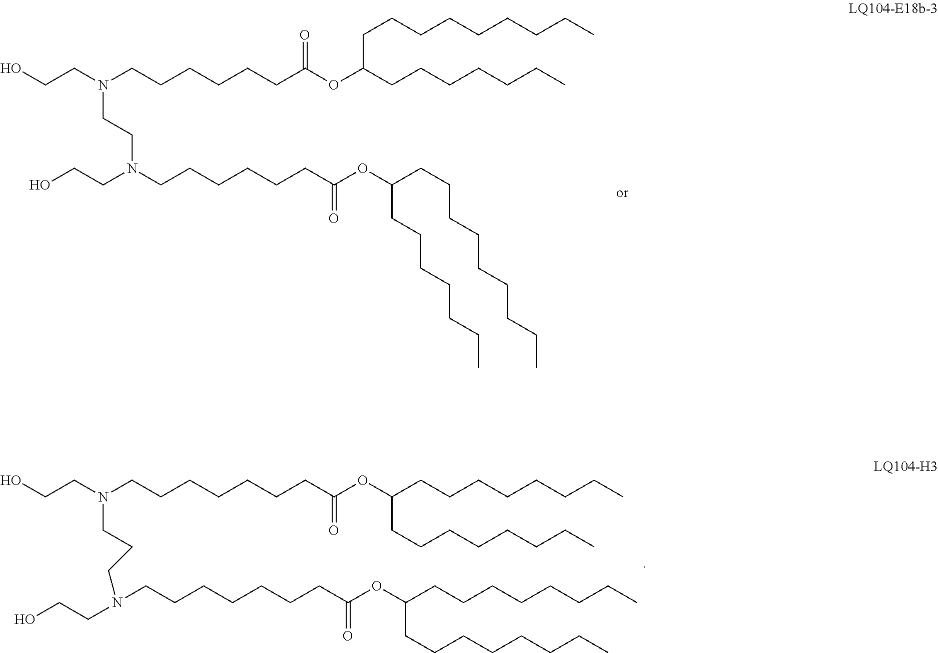 [SMILES: CCCCCCCCCC(CCCCCCC)OC(=O)CCCCCCN(CCO)CCN(CCO)CCCCCCC(=O)OC(CCCCCCC)CCCCCCCCC ; CCCCCCCCC(CCCCCCCC)OC(=O)CCCCCCCN(CCO)CCCN(CCO)CCCCCCCC(=O)OC(CCCCCCCC)CCCCCCCC]

The present invention further provides a preparation method of the nitrogen-containing chain compound represented by the formula (I), including the following steps: performing, in a solvent and in the presence of a base and an iodized salt, a coupling reaction represented by the following formula on a compound represented by a formula (I-1) and a compound represented by a formula (I-2);

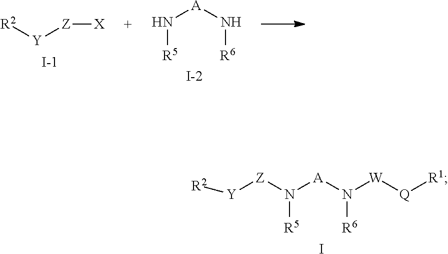

wherein, X is halogen, A is C₂-C₆ alkylene, and Y, Q, Z, W, R⁵, R⁶, R¹ and R² are as previously described; and Y is the same as Q, R¹ is the same as R², and Z is the same as W.

In the coupling reaction, the halogen may be fluorine, chlorine, bromine, or iodine, such as bromine.

In the coupling reaction, a molar ratio of the compound represented by the formula (I-2) to the compound represented by the formula (I-2) may be 1:(1-3), such as 1:2.6.

In the coupling reaction, the base may be a conventional base in the art. The base may be basic carbonate (a cation in the salt is an alkali metal ion, and an anion is a carbonate), such as K₂CO₃.

In the coupling reaction, a molar ratio of the compound represented by the formula (I-2) to the base may be 1:(1-5), such as 1:3.5.

In the coupling reaction, the solvent may be a conventional solvent in the art, and the solvent may be an ether solvent or/and a nitrile solvent. The ether solvent may be methyl tert-butyl ether. The nitrile solvent may be acetonitrile. A volume ratio of the nitrile solvent to the ether solvent may be 1:1.

In the coupling reaction, a mass-to-volume ratio of the compound represented by the formula (I-2) to the solvent may be in a range from 10 mg/mL to 50 mg/mL, such as 16 mg/mL.

In the coupling reaction, the iodized salt may be a conventional iodized salt in the art. The iodized salt may be a basic iodized salt, such as KI.

In the coupling reaction, a molar ratio of the compound represented by the formula (I-2) to the iodized salt may be 1:(1-2), such as 1:1.2.

In the coupling reaction, a reaction temperature of the coupling reaction may be a common reaction temperature in the art, preferably in a range from 50° C. to 100° C., such as 80° C.

The present invention further provides a lipid carrier, including a substance Z, wherein the substance Z is the compound represented by the formula (I) as previously described or the pharmaceutically acceptable salt thereof.

In a certain preferred solution, the lipid carrier further includes a diluent. The diluent may be phosphate buffer saline or Tris buffer.

In a certain preferred solution, the lipid carrier further includes phospholipid.

In a certain preferred solution, the phospholipid may be conventional phospholipid in the art, which is an amphoteric auxiliary molecule that facilitates fusion of lipid particles and a cell membrane. The phospholipid may be a phospholipid molecule with a charged polar end and a fatty chain non-polar end, such as distearoyl phosphatidylcholine (DSPC), 1,2-dimyristoyl-sn-glycero-3-phosphocholine (DMPC), dioleoyl phosphorylcholine (DOPC), palmitoyl phosphorylcholine (DPPC), 1,2-distearoyl phosphatidylcholine (DSPC), nonadecanoyl phosphatidylcholine (DUPC) or palmitoyl phosphorylcholine (POPC).

In a certain preferred solution, the lipid carrier further includes PEG lipid (polyethylene glycol modified lipid).

In a certain preferred solution, the PEG lipid may be a lipid molecule modified with a polyethylene glycol hydrophilic end. The PEG lipid is preferably selected from one or more of PEG-modified phosphatidyl ethanolamine, PEG-modified phosphatidic acid, PEG-modified ceramide, PEG-modified dialkyl amine, PEG-modified diacylglycerol, and PEG-modified dialkylglycerol, such as PEG-modified dimyristoyl glycerol (DMG-PEG2000).

In a certain preferred solution, the lipid carrier further includes sterol.

In a certain preferred solution, the sterol may be conventional sterol in the art, and includes animal, plant, or fungal sterols. The sterol is selected from one or more of cholesterol, sitosterol, ergosterol, campesterol, stigmasterol, brassinosteroid, tomatidine, ursolic acid, and α-tocopherol, such as cholesterol.

In a certain preferred solution, in the lipid carrier, a molar ratio of the substance Z to the sterol is (0.5-5):1, preferably (0.5-3):1, such as (0.6-2):1.

In a certain preferred solution, in the lipid carrier, the molar ratio of the substance Z to the sterol is (0.5-5):1, preferably (0.5-3):1, such as 0.68:1, 0.69:1, 0.71:1, 0.74:1, 0.76:1, 0.77:1, 0.79:1, 0.83:1, 0.84:1, 0.85:1, 0.86:1, 0.88:1, 0.89:1, 0.9:1, 0.91:1, 0.94:1, 0.99:1, 1.04:1, 1.07:1 or 1.28:1.

In a certain preferred solution, in the lipid carrier, the molar ratio of the substance Z to the sterol is (0.5-5):1, preferably (0.5-3):1, such as (0.6-2):1; and also such as, 0.66:1, 0.68:1, 0.69:1, 0.70:1, 0.71:1, 0.72:1, 0.74:1, 0.76:1, 0.77:1, 0.79:1, 0.82:1, 0.83:1, 0.84:1, 0.85:1, 0.86:1, 0.87:1, 0.88:1, 0.89:1, 0.9:1, 0.91:1, 0.92:1, 0.93:1, 0.94:1, 0.97:1, 0.99:1, 1.04:1, 1.07:1, 1.1:1, 1.16:1, 1.23:1, 1.28:1, 1.30:1, 1.32:1, 1.41:1, 1.52:1, 1.58:1, 1.64:1, 1.65:1, 1.74:1, 1.79:1 or 1.96:1.

In a certain preferred solution, in the lipid carrier, a molar ratio of the substance Z to the phospholipid is (1-15):1, preferably (2-8):1, such as (3-6):1.

In a certain preferred solution, in the lipid carrier, the molar ratio of the substance Z to the phospholipid is (1-25):1, preferably (2-25):1, such as 22.5:1, 20:1, 17.5:1, 15:1, 11.25:1, 10:1, 8.75:1, 7.5:1, 6.67:1, 5:1, 4.75:1, 4.5:1, 4:1, 3.9:1, 3.6:1, 3.3:1, 3:1, 2.86:1, 2.5:1 or 2.2:1.

In a certain preferred solution, in the lipid carrier, a molar ratio of the substance Z to the PEG lipid is (20-130):1, preferably (20-80):1, such as (20-40):1.

In a certain preferred solution, in the lipid carrier, the molar ratio of the substance Z to the PEG lipid is (16-130):1, preferably (16-80):1, such as (16-40):1; and also such as 16:1, 18:1, 20:1, 22.5:1, 25:1, 27.5:1, 28.1:1, 31.25:1 or 33.3:1.

In a certain preferred solution, in the lipid carrier, the molar ratio of the substance Z to the PEG lipid is (16-130):1, preferably (16-80):1, such as (16-40):1; and also such as 16:1, 18:1, 18.8:1, 20:1, 21.9:1, 22.5:1, 25:1, 26.9:1, 27.5:1, 28.1:1, 29.6:1, 30:1, 31.25:1, 33.3:1 or 37.5:1.

In a certain preferred solution, a molar content of the substance Z is about in a range from 30 mol % to 60 mol %.

In a certain preferred solution, the molar content of the substance Z is about in a range from 30 mol % to 60 mol %; preferably in a range from 40 mol % to 55 mol %, such as 40 mol %, 43 mol %, 45 mol %, 47.4 mol %, 50 mol % or 55 mol %.

In the present invention, a definition of the molar content is a percentage of a certain substance to a total mass of the lipid carrier, and a sum of the molar contents of all components in the lipid carrier does not exceed 100 mol %. In a certain preferred solution, a molar content of the phospholipid is about in a range from 0 mol % to 30 mol %.

In a certain preferred solution, the molar content of the phospholipid is about in a range from 0 mol % to 30 mol %; preferably in a range from 0 mol % to 18 mol %, such as 0 mol %, 2 mol %, 4 mol %, 6 mol %, 8 mol %, 10 mol %, 11 mol %, 12 mol %, 14 mol %, 16 mol %, or 18 mol %.

In a certain preferred solution, a molar content of the sterol is about in a range from 15 mol % to 55 mol %.

In a certain preferred solution, the molar content of the sterol is about in a range from 15 mol % to 60 mol %; preferably in a range from 40.4 mol % to 58.4 mol %, such as 42.4 mol %, 44.4 mol %, 46.4 mol %, 48.4 mol %, 50.4 mol %, 52.4 mol % or 56.4 mol %.

In a certain preferred solution, the molar content of the sterol is about in a range from 15 mol % to 60 mol %; preferably in a range from 40.4% mol % to 58.4 mol %, such as 40.4 mol %, 41 mol %, 42.4 mol %, 43 mol %, 43.4 mol %, 44.4 mol %, 46.4 mol %, 47.4 mol %, 48 mol %, 48.4 mol %, 49 mol %, 49.4 mol %, 49.5 mol %, 50 mol %, 50.4 mol %, 50.5 mol %, 51 mol %, 51.4 mol %, 51.5 mol %, 52 mol %, 52.25 mol %, 52.4 mol %, 52.5 mol %, 52.75 mol %, 53 mol %, 53.4 mol %, 54 mol %, 54.25 mol %, 54.4 mol %, 54.5 mol %, 54.75 mol %, 55 mol %, 56 mol %, 56.4 mol %, 56.5 mol %, 57 mol %, 57.5 mol %, 58 mol % or 58.4 mol %.

In a certain preferred solution, when the lipid carrier does not contain the phospholipid, the molar content of the sterol in the lipid carrier is about in a range from 15 mol % to 60 mol %, preferably in a range from 40.4% mol % to 58.4 mol %, such as 43 mol %, 43.4 mol %, 44.4 mol %, 46.4 mol %, 47.4 mol %, 48 mol %, 48.4 mol %, 49 mol %, 49.4 mol %, 49.5 mol %, 50 mol %, 50.4 mol %, 50.5 mol %, 51 mol %, 51.4 mol %, 51.5 mol %, 52 mol %, 52.4 mol %, 52.25 mol %, 52.5 mol %, 52.75 mol %, 53 mol %, 53.4 mol %, 54 mol %, 54.25 mol %, 54.4 mol %, 54.5 mol %, 54.75 mol %, 55 mol %, 56 mol %, 56.4 mol %, 56.5 mol %, 57 mol %, 57.5 mol %, 58 mol % or 58.4 mol %; and also such as in a range from 52.5 mol % to 54.5 mol %, and further such as in a range from 53 mol % to 54.5 mol %.

In a certain preferred solution, a molar content of the PEG lipid is about in a range from 0 mol % to 10 mol %.

In a certain preferred solution, the molar content of the PEG lipid is about in a range from 0 mol % to 10 mol %, such as in a range from 1.5 mol % to 2.5 mol %, such as 1.6 mol % or 2 mol %.

In a certain preferred solution, the molar content of the PEG lipid is about in a range from 0 mol % to 10 mol %, the molar content of the PEG lipid may be in a range from 0.5 mol % to 2.5 mol %, and may further be in a range from 0.5 mol % to 1.5 mol %, or in a range from 1.5 mol % to 2.5 mol %, such as 1.6 mol % or 2 mol %.

In a certain preferred solution, the molar content of the PEG lipid is in a range from 0 mol % to 10 mol %, such as in a range from 0.5 mol % to 2.5 mol %, and specifically, also such as 0.25 mol %, 0.5 mol %, 0.75 mol %, 1 mol %, 1.5 mol %, 1.6 mol %, 2 mol %, 2.5 mol %, 3 mol %, 3.5 mol %, 4 mol %, or 5 mol %; further in a range from 0.5 mol % to 2 mol %; it may further be in a range from 0.5 mol % to 1.5 mol %, or in a range from 1.5 mol % to 2.5 mol %; and it may further be in a range from 1.6 mol % or 2 mol %.

In a certain preferred solution, when the lipid carrier does not contain the phospholipid, or when a content of the phospholipid is below 4 mol %, a molar content of the PEG lipid is about in a range from 0 mol % to 10 mol %, specifically, such as 0.25 mol %, 0.5 mol %, 0.75 mol %, 1 mol %, 1.5 mol %, 1.6 mol %, 2 mol %, 2.5 mol %, 3 mol %, 3.5 mol %, 4 mol % or 5 mol %; such as in a range from 0.25 mol % to 3 mol %, further in a range from 0.5 mol % to 2.5 mol %, and further in a range from 0.5 mol % to 2 mol %.

In a certain preferred solution, the lipid carrier consists of the substance Z, the diluent, the phospholipid, the PEG lipid and the sterol.

In a certain preferred solution, the lipid carrier consists of the substance Z, the phospholipid, the PEG lipid and the sterol.

In a certain preferred solution, the lipid carrier consists of the substance Z, the diluent, the PEG lipid and the sterol.

In a certain preferred solution, the lipid carrier consists of the substance Z, the PEG lipid and the sterol.

In a certain preferred solution, the lipid carrier does not contain the phospholipid.

In a certain preferred solution, when the lipid carrier does not contain the phospholipid, in the lipid carrier, a molar ratio of the substance Z to the sterol may be (0.6-2):1, preferably 0.68:1, 0.69:1, 0.7:1, 0.77:1, 0.85:1, 0.86:1, 1.04:1 or 1.28:1.

In a certain preferred solution, when the lipid carrier does not contain the phospholipid, in the lipid carrier, the molar ratio of the substance Z to the sterol is (0.6-2):1, such as 0.68:1, 0.69:1, 0.70:1, 0.71:1, 0.72:1, 0.74:1, 0.76:1, 0.77:1, 0.79:1, 0.82:1, 0.83:1, 0.84:1, 0.85:1, 0.86:1, 0.87:1, 0.88:1, 0.89:1, 0.9:1, 0.91:1, 0.92:1, 0.93:1, 0.94:1, 0.97:1, 0.99:1, 1.04:1, 1.07:1, 1.1:1, 1.16:1, 1.23:1, 1.28:1, 1.30:1, 1.41:1, 1.52:1 or 1.58:1.

In a certain preferred solution, when the lipid carrier does not contain the phospholipid, in the lipid carrier, the molar ratio of the substance Z to the PEG lipid may be (16-35):1, preferably 16:1, 18:1, 20:1, 22.5:1, 25:1, 27.5:1 or 28.1:1.

In a certain preferred solution, when the lipid carrier does not contain the phospholipid, in the lipid carrier, the molar ratio of the substance Z to the PEG lipid may be (16-35):1, also such as 16:1, 18:1, 20:1, 21.9:1, 22.5:1, 25:1, 26.9:1, 27.5:1, 28.1:1, 29.6:1 or 30:1.

The present invention further provides a lipid nanoparticle, including a therapeutic agent and/or a prophylactic agent and the aforementioned lipid carrier.

In a certain preferred solution, the therapeutic agent and/or the prophylactic agent may be one or two or more nucleic acids. The nucleic acid may be conventional nucleic acid in the art. The therapeutic agent and/or the prophylactic agent may be single-stranded deoxyribonucleic acid (DNA), double-stranded DNA, small interfering RNA (siRNA), asymmetric double-stranded small interfering RNA (aiRNA), microRNA (miRNA), small hairpin RNA (shRNA), circular RNA (circRNA), transfer RNA (tRNA), messenger RNA (mRNA), and other forms of nucleic acid molecules known in the art, preferably mRNA, such as firefly luciferase (Fluc) mRNA or SARS-CoV-2 spike protein (Spike) mRNA.

In a certain preferred solution, a nitrogen-to-phosphorus ratio in the lipid nanoparticle may be in a range from 2:1 to 30:1, and the nitrogen-to-phosphorus ratio of a composition refers to a ratio of the mole number of ionizable nitrogen atoms in one or more ionizable lipid compounds to the mole number of a phosphate group in RNA, preferably in a range from 2:1 to 20:1, such as in a range from 3:1 to 20:1, and also such as in a range from 3:1 to 16:1.

In a certain preferred solution, in the lipid nanoparticle, a mass ratio of the lipid carrier to the therapeutic agent and/or the prophylactic agent may be in a range from (3-80):1, preferably in a range from (6-60):1.

In a certain preferred solution, a particle size (average particle size) of the lipid nanoparticle may be in a range from 10 nm to 200 nm, preferably in a range from 40 nm to 150 nm, such as in a range from 60 nm to 150 nm.

In a certain preferred solution, the particle size (average particle size) of the lipid nanoparticle may be in a range from 10 nm to 200 nm, preferably in a range from 40 nm to 150 nm, such as in a range from 60 nm to 150 nm, and also such as in a range from 50 nm to 150 nm.

In a certain preferred solution, in the lipid nanoparticle, the lipid carrier encapsulates the therapeutic agent and/or the prophylactic agent.

The present invention further provides a composition, including a substance Z, wherein the substance Z is the compound represented by the formula (I) as previously described or a pharmaceutically acceptable salt thereof.

In a certain preferred solution, the composition further includes one or more of a diluent, phospholipid, PEG lipid, sterol, and a therapeutic agent and/or a prophylactic agent.

In a certain preferred solution, in the composition, the diluent, the phospholipid, the PEG lipid, the sterol, and the therapeutic agent and/or the prophylactic agent are as previously described.

In a certain preferred solution, in the composition, the substance Z forms the lipid carrier as previously described with one or more of the diluent, the phospholipid, the PEG lipid, and the sterol.

In a certain preferred solution, in the composition, the lipid carrier forms the lipid nanoparticle as previously described with the therapeutic agent and/or the prophylactic agent. In a certain preferred solution, in the composition, an encapsulation efficiency of the therapeutic agent and/or the prophylactic agent is at least 50%, preferably at least 70%.

In a certain preferred solution, in the composition, a polymer dispersity index of the composition is not higher than 0.5, such as not higher than 0.3.

Unless otherwise specified, terms used in the present invention have the following meanings: a term “one or more” refers to one, two or three.

A term “halogen” refers to fluorine, chlorine, bromine, or iodine.

A term “pharmaceutically acceptable” refers to being relatively non-toxic, safe, and suitable for patient use.

A term “pharmaceutically acceptable salt” refers to a salt obtained by reacting a compound with pharmaceutically acceptable acid or base. When the compound contains relatively acidic functional groups, a base addition salt may be obtained in a suitable inert solvent in a manner of contacting a sufficient amount of pharmaceutically acceptable base with the compound. The pharmaceutically acceptable base addition salts include, but are not limited to a sodium salt, a potassium salt, a calcium salt, an aluminum salt, a magnesium salt, a bismuth salt, an ammonium salt, etc. When the compound contains relatively basic functional groups, an acid addition salt may be obtained in a suitable inert solvent in a manner of contacting a sufficient amount of pharmaceutically acceptable acid with the compound. The pharmaceutically acceptable acid addition salts include, but are not limited to: hydrochloride, sulfate, mesylate, etc. Please refer to Handbook of Pharmaceutical Salts: Properties, Selection, and Use (P. Heinrich Stahl, Camille G. Wermuth, 2011, 2nd Revised Edition) for details.

“ [symbol] ” in a structural fragment refers to the connection between the structural fragment and the rest of the molecule through this site. For example,

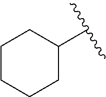 [SMILES: *C1CCCCC1]

refers to cyclohexyl.

“—” at an end of the group refers to the connection between the group and the rest of the molecule through this site. For example, CH₃—C(═O)— refers to acetyl.

A term “alkyl” refers to saturated monovalent hydrocarbyl with a specified number of carbon atoms (e.g., C₁-C₆), either straight or branched. The alkyl includes, but is not limited to methyl, ethyl, n-propyl, isopropyl, n-butyl, isobutyl, sec-butyl, tert butyl, n-pentyl, n-hexyl, etc.

A term “alkylene” is a divalent group that is connected to the rest of the molecule through two single bonds, with the remaining definition the same as the term “alkyl”.

A term “alkoxy” refers to a group R^{X}—O—, and the definition of R^{X} is the same as the term “alkyl”. Alkoxy includes, but is not limited to: methoxy, ethoxy, n-propoxy, isopropoxy, etc.

On the basis of not violating common knowledge in the art, the above preferred conditions may be arbitrarily combined to obtain various preferred examples of the present invention.

Reagents and raw materials used in the present invention are commercially available.

The positive progressive effect of the present invention is that present invention provides the nitrogen-containing chain compound represented by the formula (I), which has a novel structure and may be used for preparing the lipid nanoparticle. The lipid nanoparticle containing the nitrogen-containing chain compound represented by the formula (I) has a low polymer dispersity index and may efficiently deliver mRNA. The nitrogen-containing chain compound of the present invention can still exhibit good properties and delivery ability even when a phospholipid component is as low as 4 mol % or less when used to prepare an LNP preparation. Further, when the nitrogen-containing chain compound of the present invention is used to prepare the LNP preparation, the LNP preparation prepared under a condition of low PEG lipid still exhibits good properties and delivery ability, thereby reducing the risk of affecting efficacy and safety by high PEG lipid.

### BRIEF DESCRIPTION OF THE DRAWINGS

FIG. 1 is a diagram of results of nucleic acid gel electrophoresis of each LNP preparation prepared in Example 1.

FIG. 2 is a chemiluminescence intensity measured after co-culturing 293FT cells in Example 2 with an LNP preparation prepared in Example 1 for 18-24 hours.

FIG. 3 and FIG. 4A are total in-vivo bioluminescence measured at different times after intravenous injection administration of LNP preparations LQ104-1 to LQ104-8 prepared in Example 1 into mice in Example 3.

FIG. 4B is a total in-vivo antibody titer measured after intramuscular injection of an LNP preparation LQ104-9, LQ104-10, or LQ107 into mice in Example 3.

FIG. 5A to FIG. 5D are total in-vivo bioluminescence or total luminescence at an administration site measured at different times after intravenous injection administration (FIG. 5A and FIG. 5B) or intramuscular injection administration (FIG. 5C and FIG. 5D) of each LNP preparation in Example 4 into mice.

FIG. 6A to FIG. 6C are total in-vivo bioluminescence measured at different times after intravenous injection administration of each LNP preparation in Example 5 into mice.

FIG. 7A and FIG. 7B are total in-vivo bioluminescence measured at different times after intravenous injection administration of each LNP preparation in Example 6 into mice.

FIG. 8 is total in-vivo bioluminescence measured at different times after intravenous injection administration of each LNP preparation in Example 7 into mice.

FIG. 9A and FIG. 9B are total in-vivo bioluminescence measured at different times after intravenous injection administration of each LNP preparation in Example 8 into mice.

FIG. 10A to FIG. 10C are total in-vivo bioluminescence measured at different times after intravenous injection administration of each LNP preparation in Example 10 into mice.

FIG. 11A to FIG. 11C are total in-vivo bioluminescence measured at different times after intravenous injection administration of each LNP preparation in Example 11 into mice.

FIG. 12A to FIG. 12D are total in-vivo bioluminescence or total luminescence at an administration site measured at different times after intravenous injection administration (FIG. 12A and FIG. 12B) or intramuscular injection administration (FIG. 12C and FIG. 12D) of each LNP preparation in Example 12 into mice.

FIG. 13A and FIG. 13B are total in-vivo bioluminescence measured at different times after intravenous injection administration of each LNP preparation in Example 13 into mice.

### DETAILED DESCRIPTION OF THE PREFERRED EMBODIMENT

The present invention is further explained through examples, but the present invention is not limited to the scope of the examples. The experimental methods without specific conditions specified in the following examples shall be selected according to conventional methods and conditions, or according to the product manual.

### Preparation Example 1 Preparation of Compound LQ104

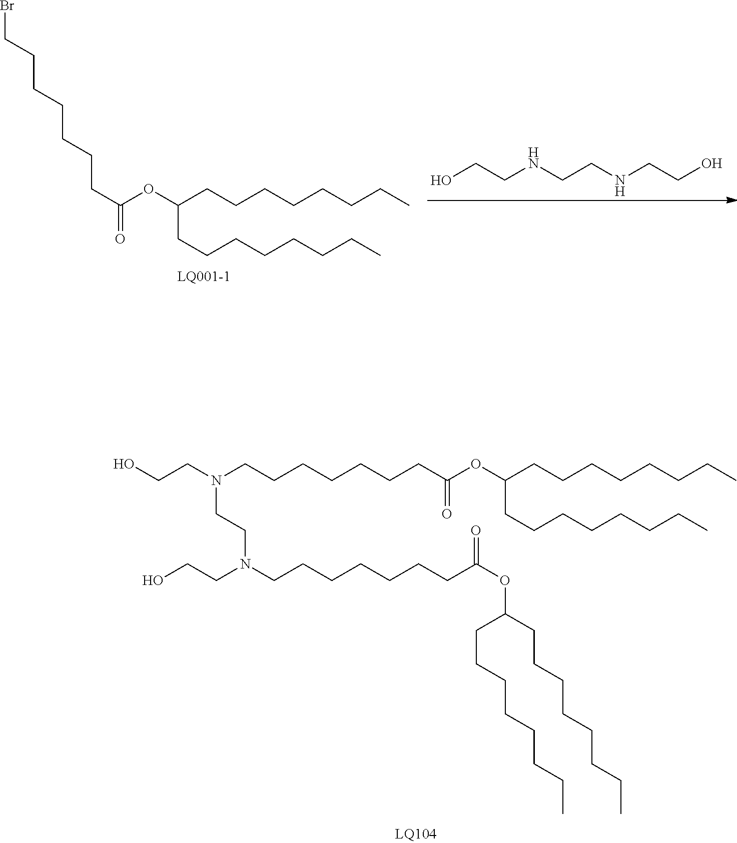

### Preparation of LQ104

### Material Ratio:

| | Molecular | Feed | | Inventory | | |
|---|---|---|---|---|---|---|
| Material name | weight | ratio | | rating | | mmol |
| LQ001-1 | 461.5 | 2.6 | eq | 4 | g | 8.5 |
| N,N′-bis(2- | 148 | 1 | eq | 487 | mg | 3.3 |
| hydroxyethyl)ethylenediamine | | | | | | |
| K₂CO₃ | 138 | 3.5 | eq | 1.6 | g | 11.5 |
| KI | 166 | 1.2 | eq | 655 | mg | 4.0 |

| | | | | | |
|---|---|---|---|---|---|
| Acetonitrile | — | — | 15 | ml | — |
| Methyl tert-butyl ether | — | — | 15 | ml | — |

### Operation Process:

LQ001-1, N,N′-bis(2-hydroxyethyl)ethylenediamine, K₂CO₃, KI, methyl tert-butyl ether, and acetonitrile were added to a reaction flask, heated to 80° C., and stirred for 12 hours. TLC (DCM:MeOH=10:1) showed complete reaction.

### Postprocessing:

A reaction solution was filtered and then subjected to rotary drying, and 1.1 g of colorless oil was obtained after purification by column chromatography.

¹HNMR (400 MHz, CDCl₃) δ:4.86 (p, 2H), 3.65-3.59 (m, 4H), 2.68-2.59 (m, 8H), 2.57-2.49 (m, 4H), 2.27 (t, 4H), 1.61 (p, 5H), 1.49 (dt, 12H), 1.28 (d, 61H), 0.87 (t, 12H).

### Preparation Example 2 Preparation of Compound LQ107

### Reaction Formula:

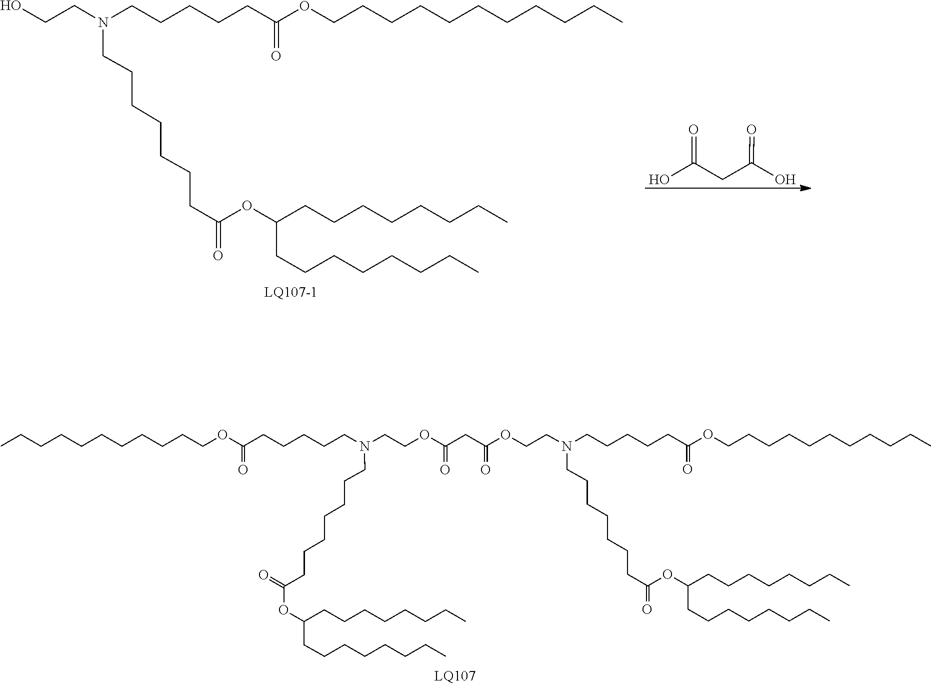

### Material Ratio:

| | Molecular | Feed | | Inventory | | |
|---|---|---|---|---|---|---|
| Material name | weight | ratio | | rating | | mmol |
| LQ107-1 | 709 | 2.1 | eq | 1 | g | 1.41 |
| Propanedioic acid | 104 | 1 | eq | 70 | mg | 0.67 |
| DCC | 206 | 2.5 | eq | 350 | mg | 1.68 |
| DMAP | 122 | 0.2 | eq | 20 | mg | 0.14 |

| | | | | | |
|---|---|---|---|---|---|
| DCM | — | — | 20 | ml | — |

### Operation Process:

LQ107-1, propanedioic acid, DCC, DMAP, and DCM were added to a reaction flask and stirred at a room temperature for 12 hours. TLC (DCM:MeOH=20:1) showed complete reaction.

### Postprocessing:

A reaction solution was filtered through diatomaceous earth and then subjected to rotary drying.

After purification by column chromatography, 700 mg of colorless oil was obtained with a yield of 70%.

¹HNMR (400 MHz, CDCl₃) δ:4.86 (p, 2H), 4.07 (dt, 8H), 2.66 (t, 4H), 2.49-2.38 (m, 8H), 2.28 (q, 8H), 2.05 (s, 5H), 1.62 (q, 17H), 1.83-1.36 (m, 17H), 1.27 (d, 90H), 0.87 (t, 18H).

### Preparation Example 3 Preparation of Compound LQ104-E15b-1

### Preparation of E15b-1

### Reaction Formula:

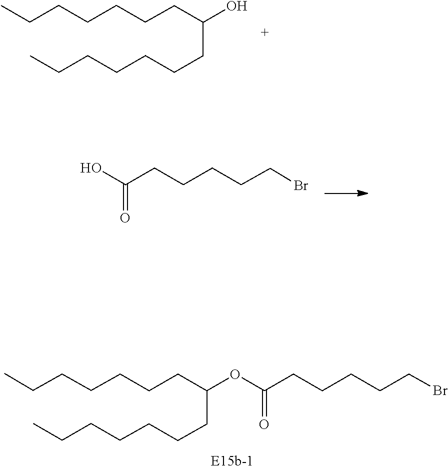 [SMILES: CCCCCCCC(O)CCCCCCC ; O=C(O)CCCCCBr ; CCCCCCCC(CCCCCCC)OC(=O)CCCCCBr]

| | Molecular | Feed | | Inventory | | |
|---|---|---|---|---|---|---|
| Material name | weight | ratio | | rating | | mmol |
| 8-Pentadecanol | 228.42 | 1 | eq | 11.4 | g | 50 |
| 6-Bromohexanoic acid | 195.06 | 1.1 | eq | 10.7 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

6-Bromohexanoic acid, DCC, DMAP, and DCM were added to a 1 L reaction flask, followed by 8-pentadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 15 g of colorless oil was obtained after purification by column chromatography.

### Preparation of LQ104-E15b-1

### Reaction Formula:

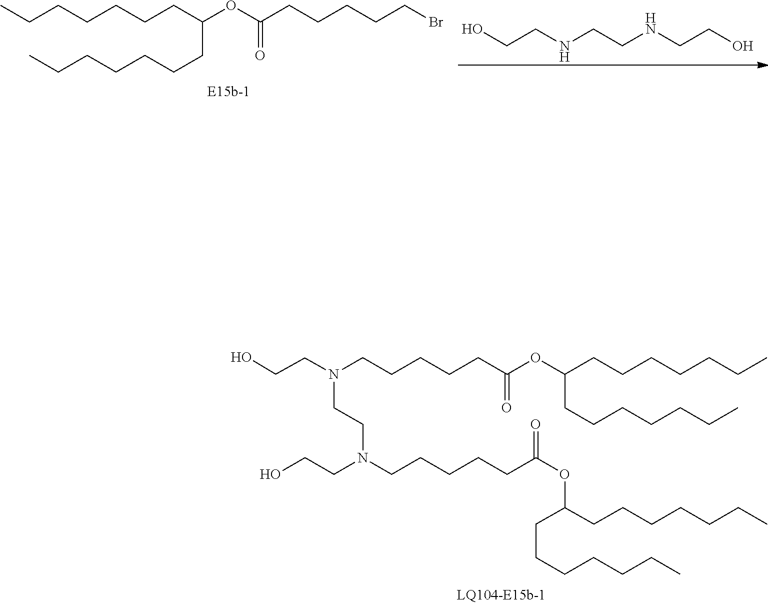

### Material Ratio:

| | Molecular | Feed | | Inventory | | |
|---|---|---|---|---|---|---|
| Material name | weight | ratio | | rating | | mmol |
| E15b-1 | 405.5 | 2.5 | eq | 2 | g | 5 |
| N,N′-bis(2- | 148 | 1 | eq | 300 | mg | 2 |
| hydroxyethyl)ethylenediamine | | | | | | |
| (CAS: 4439-20-7) | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E15b-1, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 800 mg of colorless oil was obtained after purification by column chromatography.

¹H NMR (600 MHz, CDCl₃) δ:4.86 (p, J=6.2 Hz, 2H), 3.62 (t, J=4.9 Hz, 4H), 2.67-2.59 (m, 8H), 2.55 (t, J=8.0 Hz, 4H), 2.28 (t, J=7.5 Hz, 4H), 1.64 (p, J=7.5 Hz, 4H), 1.50 (tt, J=8.5, 4.5 Hz, 12H), 1.34-1.20 (m, 46H), 0.87 (t, J=7.0 Hz, 12H).

### Preparation Example 4 Preparation of Compound LQ104-E15b-2

### Preparation of E15b-2

### Reaction Formula:

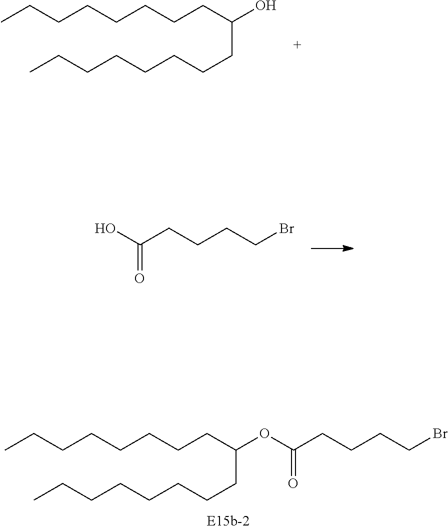 [SMILES: CCCCCCCCC(O)CCCCCCCC ; O=C(O)CCCCBr ; CCCCCCCCC(CCCCCCCC)OC(=O)CCCCBr]

### Material Ratio:

| | Molecular | Feed | | Inventory | | |
|---|---|---|---|---|---|---|
| Material name | weight | ratio | | rating | | mmol |
| 9-Heptadecanol | 256.47 | 1 | eq | 12.8 | g | 50 |
| 5-Bromovaleric acid | 181 | 1.1 | eq | 9.96 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

5-Bromovaleric acid, DCC, DMAP, and DCM were added to a 1 L reaction flask, followed by 9-heptadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 13.8 g of colorless oil was obtained after purification by column chromatography.

### Preparation of LQ104-E15b-2

### Reaction Formula:

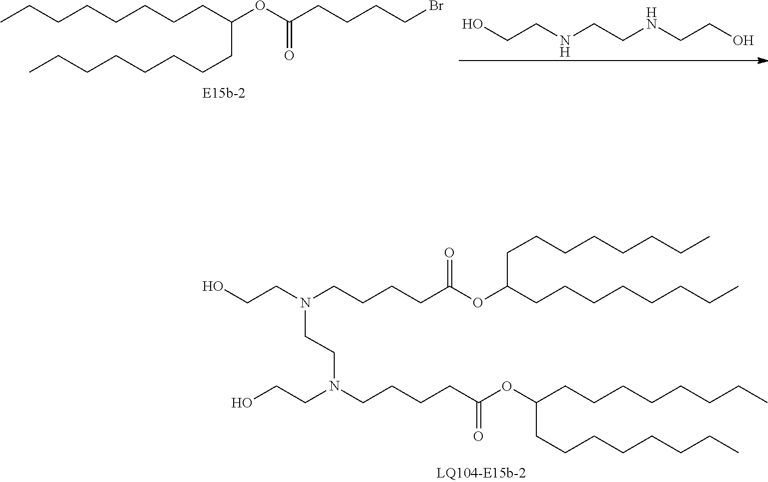 [SMILES: CCCCCCCCC(CCCCCCCC)OC(=O)CCCCBr ; OCCNCCNCCO ; CCCCCCCCC(CCCCCCCC)OC(=O)CCCCN(CCO)CCN(CCO)CCCCC(=O)OC(CCCCCCCC)CCCCCCCC]

### Material Ratio:

| | Molecular | Feed | | Inventory | | |
|---|---|---|---|---|---|---|
| Material name | weight | ratio | | rating | | mmol |
| E15b -2 | 419.5 | 2.5 | eq | 2.1 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E15b-2, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 760 mg of colorless oil was obtained after purification by column chromatography.

¹H NMR (600 MHz, CDCl₃) δ: 4.85 (p, J=6.2 Hz, 2H), 3.70 (t, J=4.9 Hz, 4H), 2.85-2.69 (m, 12H), 2.32 (t, J=7.0 Hz, 4H), 1.65-1.54 (m, 9H), 1.49 (q, J=6.4 Hz, 8H), 1.25 (d, J=9.9 Hz, 50H), 0.87 (t, J=7.0 Hz, 12H).

### Preparation Example 5 Preparation of Compound LQ104-E15b-3

### Preparation of E15b-3

### Reaction Formula:

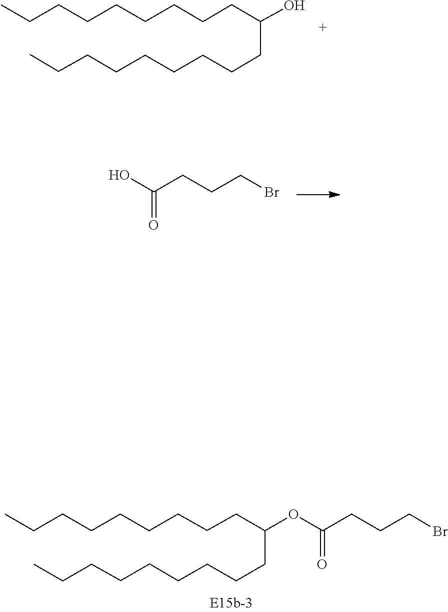 [SMILES: CCCCCCCCCC(O)CCCCCCCCC ; O=C(O)CCCBr ; CCCCCCCCCC(CCCCCCCCC)OC(=O)CCCBr]

### Material Ratio:

| | Molecular | Feed | | Inventory | | |
|---|---|---|---|---|---|---|
| Material name | weight | ratio | | rating | | mmol |
| 10-Nonadecanol | 284.5 | 1 | eq | 14.2 | g | 50 |
| 4-Bromobutyric acid | 167 | 1.1 | eq | 9.2 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

4-Bromobutyric acid, DCC, DMAP, and acetonitrile were added to a 1 L reaction flask, followed by 10-nonadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 15.3 g of colorless oil was obtained after purification by column chromatography.

### Preparation of LQ104-E15b-3

### Reaction Formula:

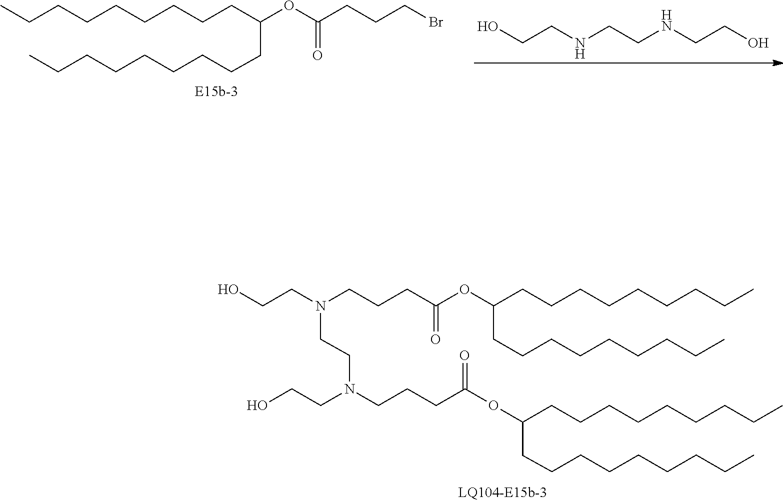 [SMILES: CCCCCCCCCC(CCCCCCCCC)OC(=O)CCCBr ; OCCNCCNCCO ; CCCCCCCCCC(CCCCCCCCC)OC(=O)CCCN(CCO)CCN(CCO)CCCC(=O)OC(CCCCCCCCC)CCCCCCCCC]

### Material Ratio:

| | Molecular | Feed | | Inventory | | |
|---|---|---|---|---|---|---|
| Material name | weight | ratio | | rating | | mmol |
| E15b-3 | 433.5 | 2.5 | eq | 2.17 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E15b-3, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 820 mg of colorless oil was obtained after purification by column chromatography.

¹H NMR (600 MHz, CDCl₃) δ:4.85 (p, J=6.2 Hz, 2H), 3.69 (t, J=4.8 Hz, 4H), 2.76 (s, 8H), 2.66 (t, J=8.2 Hz, 4H), 2.27 (t, J=7.5 Hz, 4H), 1.61 (p, J=7.3 Hz, 4H), 1.50 (dq, J=12.3, 6.4, 5.3 Hz, 12H), 1.35-1.20 (m, 54H), 0.87 (t, J=7.0 Hz, 12H).

### Preparation Example 6 Preparation of Compound LQ104-E16b-1

### Preparation of E16b-1

### Reaction Formula:

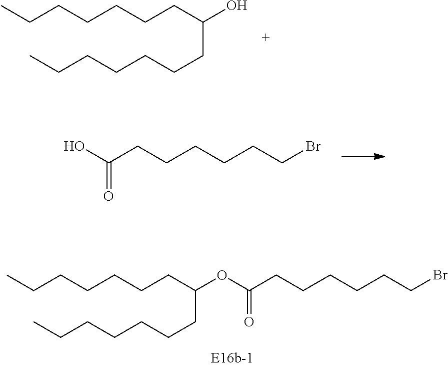 [SMILES: CCCCCCCC(O)CCCCCCC ; O=C(O)CCCCCCBr ; CCCCCCCC(CCCCCCC)OC(=O)CCCCCCBr]

### Material Ratio:

| | Molecular | | | Inventory | | |
|---|---|---|---|---|---|---|
| Material name | weight | Feed ratio | | rating | | mmol |
| 8-Pentadecanol | 228.42 | 1 | eq | 11.4 | g | 50 |
| 7-Bromoheptanoic acid | 209 | 1.1 | eq | 11.5 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

7-Bromoheptanoic acid, DCC, DMAP, and DCM were added to a 1 L reaction flask, followed by 8-pentadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 14.5 g of colorless oil was obtained after purification by column chromatography.

### Preparation of LQ104-E16b-1

### Reaction Formula:

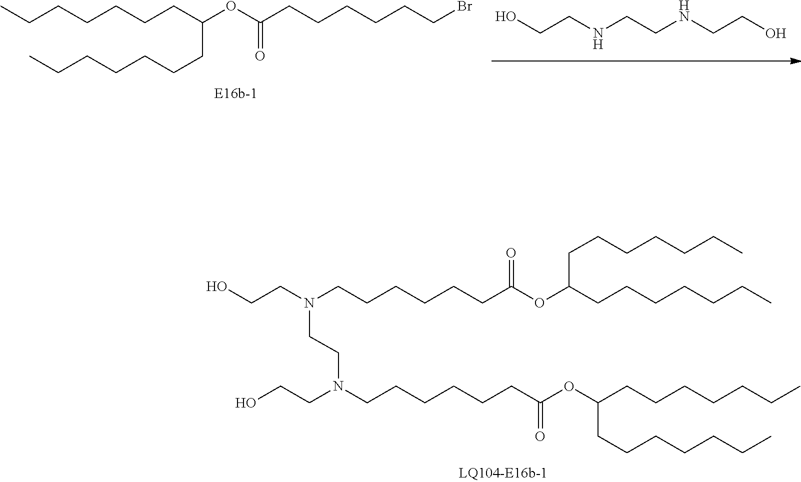 [SMILES: CCCCCCCC(CCCCCCC)OC(=O)CCCCCCBr ; OCCNCCNCCO ; CCCCCCCC(CCCCCCC)OC(=O)CCCCCCN(CCO)CCN(CCO)CCCCCCC(=O)OC(CCCCCCC)CCCCCCC]

### Material Ratio:

| | Molecular | | | Inventory | | |
|---|---|---|---|---|---|---|
| Material name | weight | Feed ratio | | rating | | mmol |
| E16b-1 | 419.5 | 2.5 | eq | 2.1 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E16b-1, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 730 mg of colorless oil was obtained after purification by column chromatography.

¹H NMR (600 MHz, CDCl₃) δ:4.85 (p, J=6.2 Hz, 2H), 3.61 (t, J=4.9 Hz, 4H), 2.67-2.60 (m, 8H), 2.54 (t, J=7.9 Hz, 4H), 2.27 (t, J=7.5 Hz, 4H), 1.61 (p, J=7.5 Hz, 4H), 1.50 (qd, J=7.6, 3.4 Hz, 12H), 1.37-1.20 (m, 50H), 0.87 (t, J=7.0 Hz, 12H).

### Preparation Example 7 Preparation of Compound LQ104-E16b-2

### Preparation of E16b-2

### Reaction Formula:

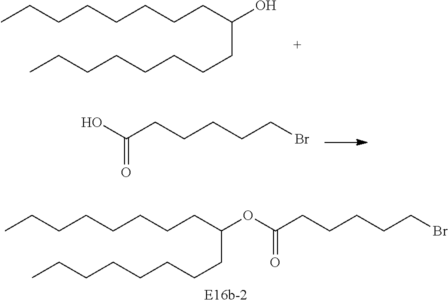 [SMILES: CCCCCCCCC(O)CCCCCCCC ; O=C(O)CCCCCBr ; CCCCCCCCC(CCCCCCCC)OC(=O)CCCCCBr]

### Material Ratio:

| | Molecular | | | Inventory | | |
|---|---|---|---|---|---|---|
| Material name | weight | Feed ratio | | rating | | mmol |
| 9-Heptadecanol | 256.47 | 1 | eq | 12.8 | g | 50 |
| 6-Bromohexanoic acid | 195.06 | 1.1 | eq | 10.7 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

6-Bromohexanoic acid, DCC, DMAP, and DCM were added to a 1 L reaction flask, followed by 9-heptadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 14.4 g of colorless oil was obtained after purification by column chromatography.

### Preparation of LQ104-E16b-2

### Reaction Formula:

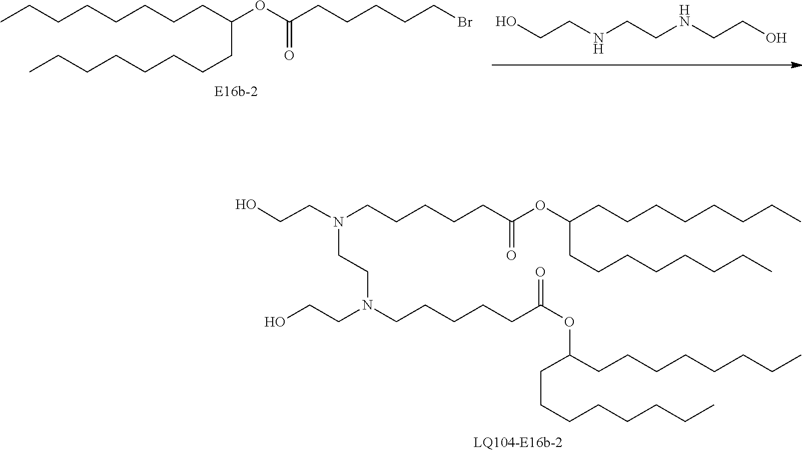 [SMILES: CCCCCCCCC(CCCCCCCC)OC(=O)CCCCCBr ; OCCNCCNCCO ; CCCCCCCCC(CCCCCCCC)OC(=O)CCCCCN(CCO)CCN(CCO)CCCCCC(=O)OC(CCCCCCCC)CCCCCCCC]

### Material Ratio:

| | Molecular | | | Inventory | | |
|---|---|---|---|---|---|---|
| Material name | weight | Feed ratio | | rating | | mmol |
| E16b-2 | 433.5 | 2.5 | eq | 2.17 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E16b-2, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 770 mg of colorless oil was obtained after purification by column chromatography.

¹H NMR (600 MHz, CDCl₃) δ:4.85 (p, J=6.2 Hz, 2H), 3.65 (t, J=4.8 Hz, 4H), 2.73-2.65 (m, 8H), 2.61 (t, J=8.1 Hz, 4H), 2.28 (t, J=7.4 Hz, 4H), 1.64 (p, J=7.5 Hz, 4H), 1.51 (dq, J=19.0, 7.3, 6.8 Hz, 12H), 1.35-1.20 (m, 54H), 0.87 (t, J=6.9 Hz, 12H).

### Preparation Example 8 Preparation of Compound LQ104-E16b-3

### Preparation of E16b-3

### Reaction Formula:

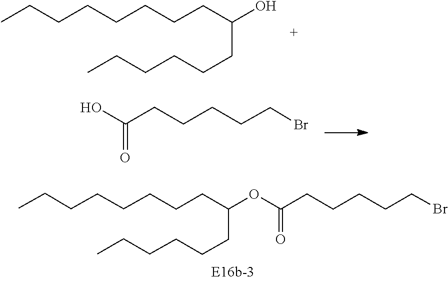

### Material Ratio:

| | Molecular | | | Inventory | | |
|---|---|---|---|---|---|---|
| Material name | weight | Feed ratio | | rating | | mmol |
| 7-Pentadecanol | 228.42 | 1 | eq | 11.4 | g | 50 |
| 6-Bromohexanoic acid | 195.06 | 1.1 | eq | 10.7 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

6-Bromohexanoic acid, DCC, DMAP, and DCM were added to a 1 L reaction flask, followed by 7-pentadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 13.3 g of colorless oil was obtained after purification by column chromatography.

### Preparation of LQ104-E16b-3

### Reaction Formula:

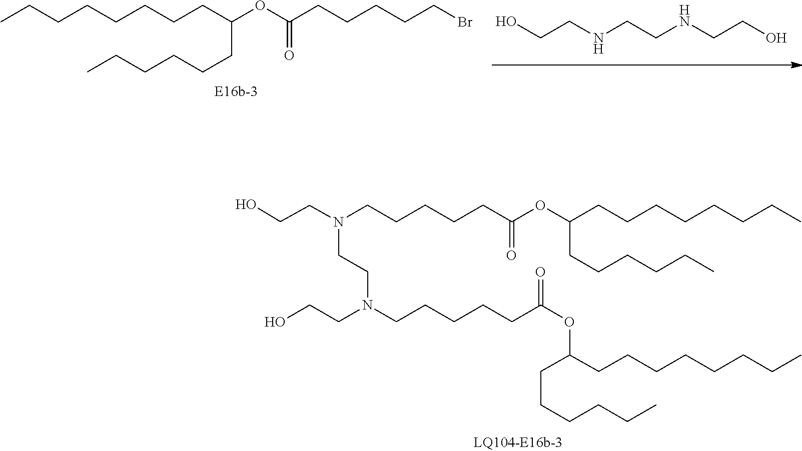 [SMILES: CCCCCCCCC(CCCCCC)OC(=O)CCCCCBr ; OCCNCCNCCO ; CCCCCCCCC(CCCCCC)OC(=O)CCCCCN(CCO)CCN(CCO)CCCCCC(=O)OC(CCCCCC)CCCCCCCC]

### Material Ratio:

| | Molecular | | | Inventory | | |
|---|---|---|---|---|---|---|
| Material name | weight | Feed ratio | | rating | | mmol |
| E16b-3 | 405.5 | 2.5 | eq | 2 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E16b-3, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 660 mg of colorless oil was obtained after purification by column chromatography.

¹H NMR (600 MHz, CDCl₃) δ:4.85 (p, J=6.3 Hz, 2H), 3.65 (t, J=4.8 Hz, 4H), 2.69 (d, J=5.2 Hz, 8H), 2.62 (d, J=8.2 Hz, 4H), 2.28 (t, J=7.4 Hz, 4H), 1.64 (p, J=7.6 Hz, 4H), 1.51 (dq, J=18.9, 6.9, 6.0 Hz, 12H), 1.35-1.18 (m, 46H), 0.87 (t, J=6.9 Hz, 12H).

### Preparation Example 9 Preparation of Compound LQ104-E16b-3R

### Preparation of E16b-3R

### Reaction Formula:

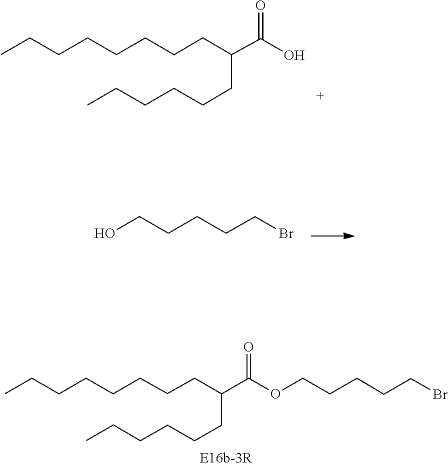 [SMILES: CCCCCCCCC(CCCCCC)C(=O)O ; OCCCCCBr ; CCCCCCCCC(CCCCCC)C(=O)OCCCCCBr]

### Material Ratio:

| | Molecular | | | Inventory | | |
|---|---|---|---|---|---|---|
| Material name | weight | Feed ratio | | rating | | mmol |
| 5-Bromopentan-1-ol | 167 | 1 | eq | 8.35 | g | 50 |
| 2-Hexyldecanoic acid | 256.43 | 1.1 | eq | 14.1 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

2-Hexyldecanoic acid, DCC, DMAP, and DCM were added to a 1 L reaction flask, followed by 5-bromopentan-1-ol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 14.2 g of colorless oil was obtained after purification by column chromatography.

### Preparation of LQ104-E16b-3R

### Reaction Formula:

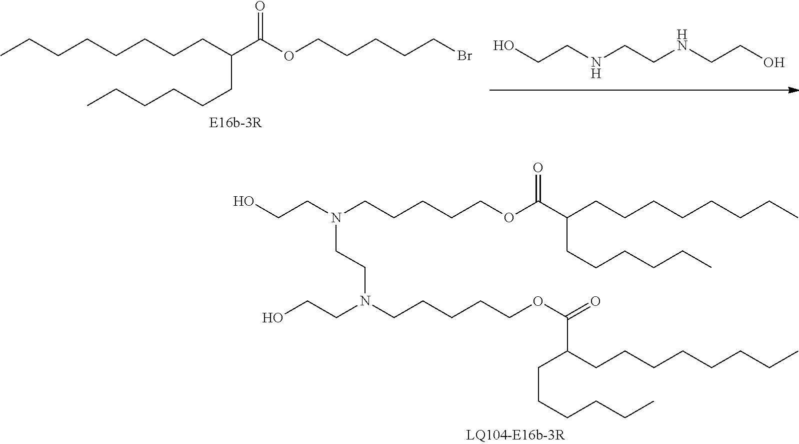 [SMILES: CCCCCCCCC(CCCCCC)C(=O)OCCCCCBr ; OCCNCCNCCO ; CCCCCCCCC(CCCCCC)C(=O)OCCCCCN(CCO)CCN(CCO)CCCCCOC(=O)C(CCCCCC)CCCCCCCC]

### Material Ratio:

| | Molecular | | | Inventory | | |
|---|---|---|---|---|---|---|
| Material name | weight | Feed ratio | | rating | | mmol |
| E16b-3R | 405.5 | 2.5 | eq | 2 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E16b-3R, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 840 mg of colorless oil was obtained after purification by column chromatography.

¹H NMR (600 MHz, CDCl₃) δ:4.06 (t, J=6.6 Hz, 4H), 3.70 (t, J=4.8 Hz, 4H), 2.73 (d, J=51.1 Hz, 12H), 2.30 (tt, J=8.8, 5.3 Hz, 2H), 1.66 (p, J=6.9 Hz, 4H), 1.57 (p, J=7.9 Hz, 8H), 1.46-1.39 (m, 4H), 1.35 (p, J=7.5 Hz, 4H), 1.32-1.19 (m, 42H), 0.87 (t, J=6.9 Hz, 12H).

### Preparation Example 10 Preparation of Compound LQ104-E17b-1

### Preparation of E17b-1

### Reaction Formula:

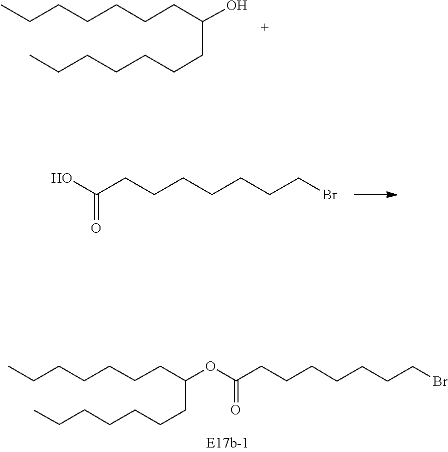 [SMILES: CCCCCCCC(O)CCCCCCC ; O=C(O)CCCCCCCBr ; CCCCCCCC(CCCCCCC)OC(=O)CCCCCCCBr]

### Material Ratio:

| | Molecular | | | Inventory | | |
|---|---|---|---|---|---|---|
| Material name | weight | Feed ratio | | rating | | mmol |
| 8-Pentadecanol | 228.42 | 1 | eq | 11.4 | g | 50 |
| 8-Bromooctanoic acid | 223 | 1.1 | eq | 12.3 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

8-Bromooctanoic acid, DCC, DMAP, and DCM were added to a 1 L reaction flask, followed by 8-pentadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 14.8 g of colorless oil was obtained after purification by column chromatography.

### Preparation of LQ104-E17b-1

### Reaction Formula:

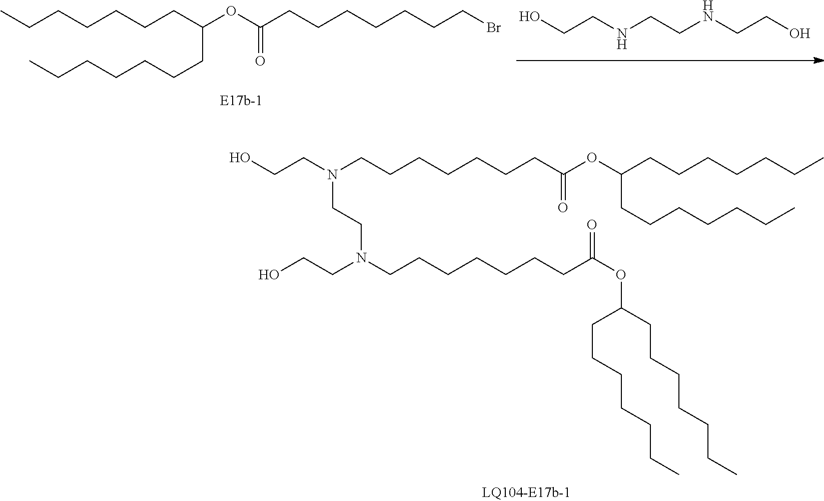

### Material Ratio:

| | Molecular | | | Inventory | | |
|---|---|---|---|---|---|---|
| Material name | weight | Feed ratio | | rating | | mmol |
| E17b-1 | 433.5 | 2.5 | eq | 2.17 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E17b-1, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 750 mg of colorless oil was obtained after purification by column chromatography.

¹H NMR (600 MHz, CDCl₃) δ:4.86 (p, J=6.2 Hz, 2H), 3.64 (t, J=4.8 Hz, 4H), 2.68 (d, J=6.8 Hz, 8H), 2.58 (t, J=8.0 Hz, 4H), 2.27 (t, J=7.5 Hz, 4H), 1.61 (p, J=7.2 Hz, 4H), 1.49 (qd, J=7.7, 5.2, 4.2 Hz, 12H), 1.36-1.20 (m, 54H), 0.87 (t, J=7.0 Hz, 12H).

### Preparation Example 11 Preparation of Compound LQ104-E17b-2

### Preparation of E17b-2

### Reaction Formula:

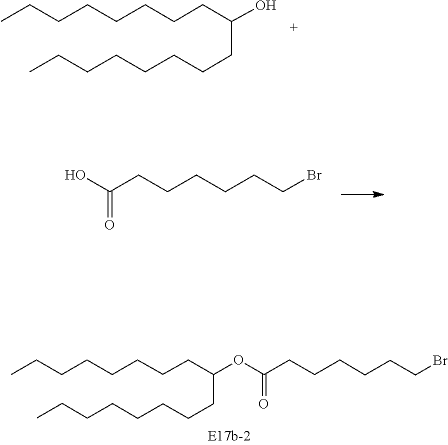 [SMILES: CCCCCCCCC(O)CCCCCCCC ; O=C(O)CCCCCCBr ; CCCCCCCCC(CCCCCCCC)OC(=O)CCCCCCBr]

### Material Ratio:

| | Molecular | | | Inventory | | |
|---|---|---|---|---|---|---|
| Material name | weight | Feed ratio | | rating | | mmol |
| 9-Heptadecanol | 256.47 | 1 | eq | 12.8 | g | 50 |
| 7-Bromoheptanoic acid | 209 | 1.1 | eq | 11.5 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

7-Bromoheptanoic acid, DCC, DMAP, and DCM were added to a 1 L reaction flask, followed by 9-heptadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 15.1 g of colorless oil was obtained after purification by column chromatography.

### Preparation of LQ104-E17b-2

### Reaction Formula:

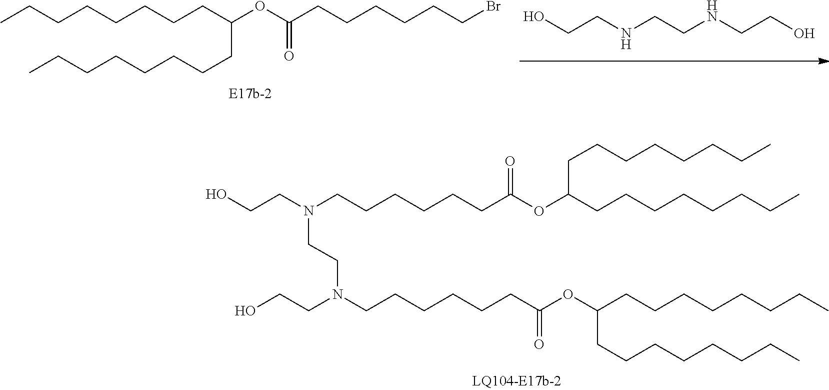 [SMILES: CCCCCCCCC(CCCCCCCC)OC(=O)CCCCCCBr ; OCCNCCNCCO ; CCCCCCCCC(CCCCCCCC)OC(=O)CCCCCCN(CCO)CCN(CCO)CCCCCCC(=O)OC(CCCCCCCC)CCCCCCCC]

### Material Ratio:

| | Molecular | | | Inventory | | |
|---|---|---|---|---|---|---|
| Material name | weight | Feed ratio | | rating | | mmol |
| E17b-2 | 447.5 | 2.5 | eq | 2.24 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E17b-2, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 680 mg of colorless oil was obtained after purification by column chromatography.

¹H NMR (600 MHz, CDCl₃) δ:4.85 (p, J=6.2 Hz, 2H), 3.67 (t, J=4.9 Hz, 4H), 2.73 (s, 8H), 2.64 (t, J=8.0 Hz, 4H), 2.27 (t, J=7.5 Hz, 4H), 1.61 (p, J=7.4 Hz, 4H), 1.51 (dd, J=13.6, 7.0 Hz, 12H), 1.38-1.19 (m, 58H), 0.87 (t, J=7.0 Hz, 12H).

### Preparation Example 12 Preparation of Compound LQ104-E17b-3

### Preparation of E17b-3

### Reaction Formula:

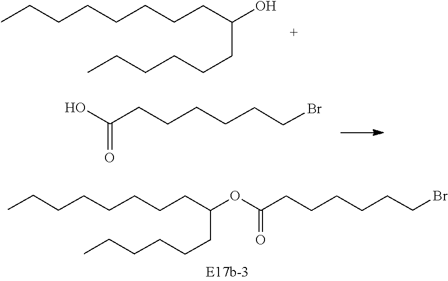

### Material Ratio:

| | Molecular | | | Inventory | | |
|---|---|---|---|---|---|---|
| Material name | weight | Feed ratio | | rating | | mmol |
| 7-Pentadecanol | 228.42 | 1 | eq | 11.4 | g | 50 |
| 7-Bromoheptanoic acid | 209 | 1.1 | eq | 11.5 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

7-Bromoheptanoic acid, DCC, DMAP, and DCM were added to a 1 L reaction flask, followed by 7-pentadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 12.8 g of colorless oil was obtained after purification by column chromatography.

### Preparation of LQ104-E17b-3

### Reaction Formula:

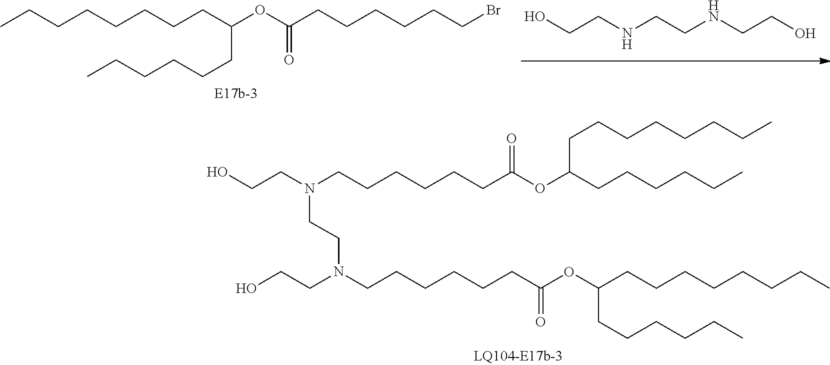 [SMILES: CCCCCCCCC(CCCCCC)OC(=O)CCCCCCBr ; OCCNCCNCCO ; CCCCCCCCC(CCCCCC)OC(=O)CCCCCCN(CCO)CCN(CCO)CCCCCCC(=O)OC(CCCCCC)CCCCCCCC]

### Material Ratio:

| | Molecular | | | Inventory | | |
|---|---|---|---|---|---|---|
| Material name | weight | Feed ratio | | rating | | mmol |
| E17b-3 | 419.5 | 2.5 | eq | 2.1 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E17b-3, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 590 mg of colorless oil was obtained after purification by column chromatography.

¹H NMR (600 MHz, CDCl₃) δ:4.86 (p, J=6.3 Hz, 2H), 3.63 (t, J=4.8 Hz, 4H), 2.66 (dd, J=9.9, 5.1 Hz, 8H), 2.56 (t, J=8.0 Hz, 4H), 2.27 (t, J=7.4 Hz, 4H), 1.62 (p, J=7.5 Hz, 4H), 1.49 (dd, J=10.3, 4.9 Hz, 12H), 1.38-1.20 (m, 50H), 0.87 (t, J=7.0 Hz, 12H).

### Preparation Example 13 Preparation of Compound LQ104-E17b-3R

### Preparation of E17b-3R

### Reaction Formula:

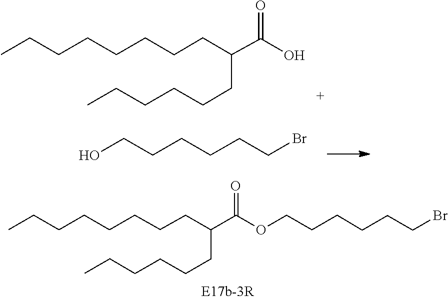

### Material Ratio:

| | Molecular | | | Inventory | | |
|---|---|---|---|---|---|---|
| Material name | weight | Feed ratio | | rating | | mmol |
| 6-Bromo-1-hexanol | 181 | 1 | eq | 9.05 | g | 50 |
| 2-Hexyldecanoic acid | 256.43 | 1.1 | eq | 14.1 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

2-Hexyldecanoic acid, DCC, DMAP, and DCM were added to a 1 L reaction flask, followed by 6-bromo-1-hexanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 14.5 g of colorless oil was obtained after purification by column chromatography.

### Preparation of LQ104-E17b-3R

### Reaction Formula:

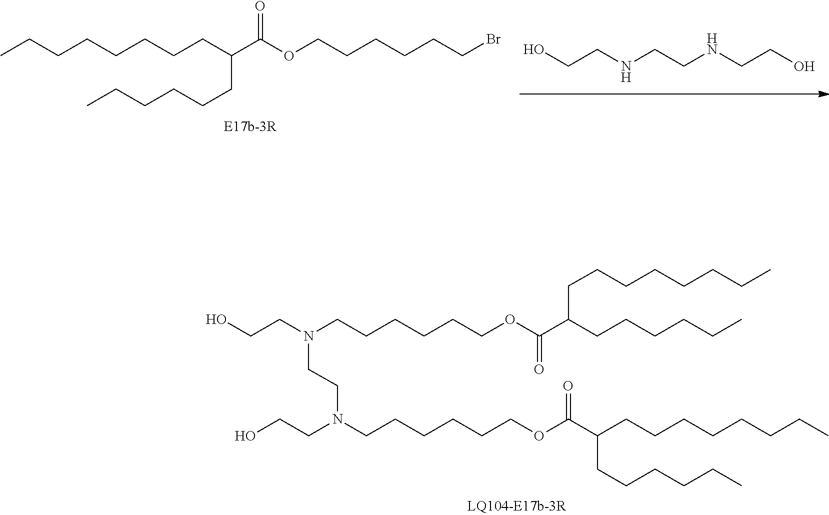 [SMILES: CCCCCCCCC(CCCCCC)C(=O)OCCCCCCBr ; OCCNCCNCCO ; CCCCCCCCC(CCCCCC)C(=O)OCCCCCCN(CCO)CCN(CCO)CCCCCCOC(=O)C(CCCCCC)CCCCCCCC]

### Material Ratio:

| | Molecular | | | Inventory | | |
|---|---|---|---|---|---|---|
| Material name | weight | Feed ratio | | rating | | mmol |
| E17b-3R | 419.5 | 2.5 | eq | 2.1 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E17b-3R, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 770 mg of colorless oil was obtained after purification by column chromatography.

¹H NMR (600 MHz, CDCl₃) δ:4.05 (t, J=6.6 Hz, 4H), 3.66 (t, J=4.8 Hz, 4H), 2.66 (d, J=52.0 Hz, 12H), 2.30 (tt, J=8.9, 5.3 Hz, 2H), 1.66-1.48 (m, 12H), 1.46-1.20 (m, 54H), 0.87 (td, J=7.0, 1.5 Hz, 12H).

### Preparation Example 14 Preparation of Compound LQ104-E17b-4

### Preparation of E17b-4

### Reaction Formula:

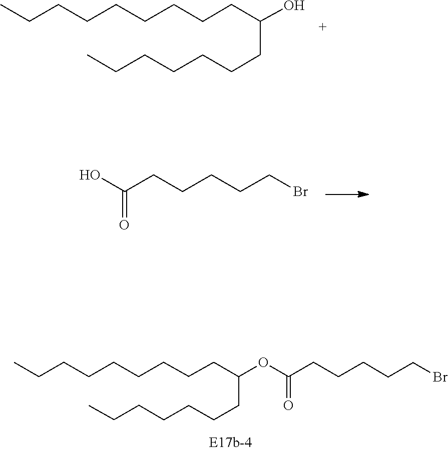 [SMILES: CCCCCCCCCC(O)CCCCCCC ; O=C(O)CCCCCBr ; CCCCCCCCCC(CCCCCCC)OC(=O)CCCCCBr]

### Material Ratio:

| | Molecular | | | Inventory | | |
|---|---|---|---|---|---|---|
| Material name | weight | Feed ratio | | rating | | mmol |
| 8-Heptadecanol | 256.47 | 1 | eq | 12.8 | g | 50 |
| 6-Bromohexanoic acid | 195.06 | 1.1 | eq | 10.7 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

6-Bromohexanoic acid, DCC, DMAP, and acetonitrile were added to a 1 L reaction flask, followed by 8-heptadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 15.2 g of colorless oil was obtained after purification by column chromatography.

### Preparation of LQ104-E17b-4

### Reaction Formula:

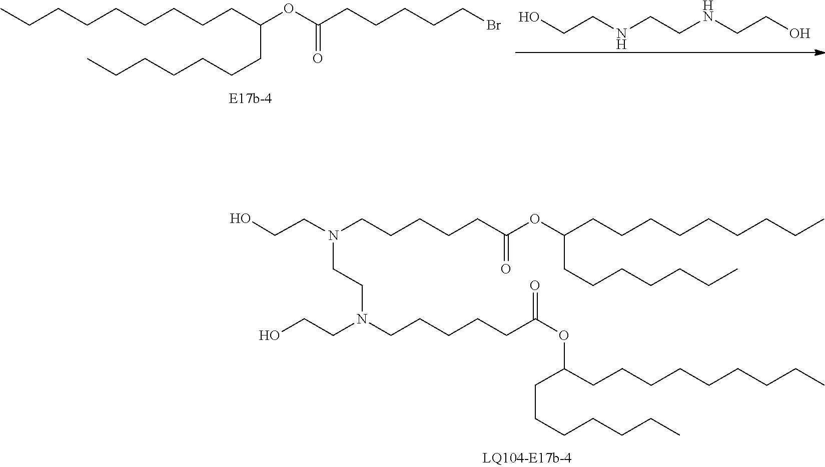

### Material Ratio:

| | Molecular | | | Inventory | | |
|---|---|---|---|---|---|---|
| Material name | weight | Feed ratio | | rating | | mmol |
| E17b-4 | 419.5 | 2.5 | eq | 2.1 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E17b-4, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 840 mg of colorless oil was obtained after purification by column chromatography.

¹H NMR (600 MHz, CDCl₃) δ:4.85 (p, J=6.3 Hz, 2H), 3.67 (t, J=4.8 Hz, 4H), 2.74 (s, 8H), 2.66 (t, J=8.0 Hz, 4H), 2.29 (t, J=7.4 Hz, 4H), 1.64 (p, J=7.5 Hz, 4H), 1.52 (dq, J=26.5, 6.9, 6.0 Hz, 12H), 1.37-1.19 (m, 54H), 0.87 (t, J=6.9 Hz, 12H).

### Preparation Example 15 Preparation of Compound LQ104-E18b-2

### Preparation of E18b-2

### Reaction Formula:

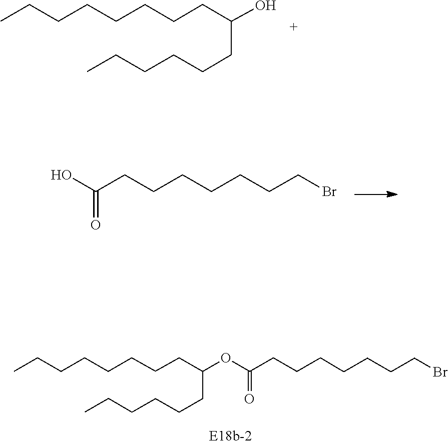 [SMILES: CCCCCCCCC(O)CCCCCC ; O=C(O)CCCCCCCBr ; CCCCCCCCC(CCCCCC)OC(=O)CCCCCCCBr]

### Material Ratio:

| | Molecular | | | Inventory | | |
|---|---|---|---|---|---|---|
| Material name | weight | Feed ratio | | rating | | mmol |
| 7-Pentadecanol | 228.42 | 1 | eq | 11.4 | g | 50 |
| 8-Bromooctanoic acid | 223 | 1.1 | eq | 12.3 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

8-Bromooctanoic acid, DCC, DMAP, and DCM were added to a 1 L reaction flask, followed by 7-pentadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 14.6 g of colorless oil was obtained after purification by column chromatography.

### Preparation of LQ104-E18b-2

### Reaction Formula:

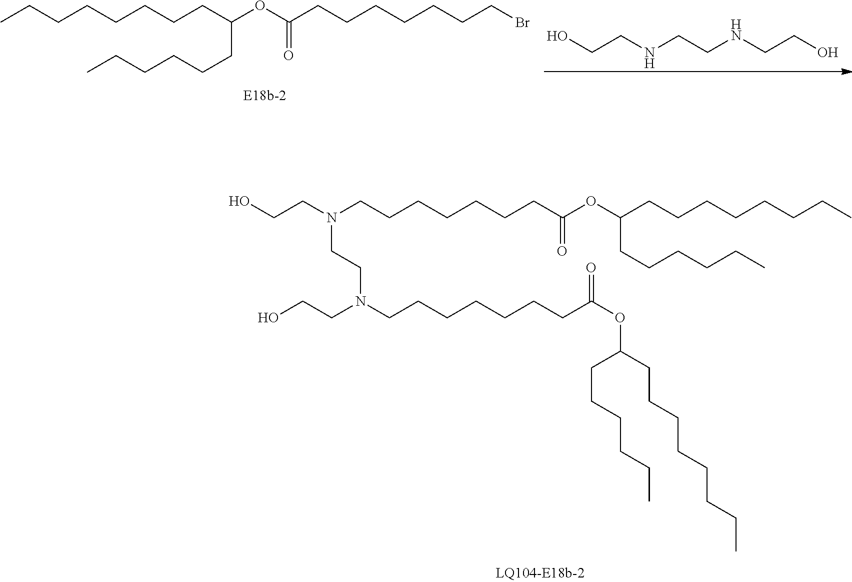

### Material Ratio:

| | Molecular | | | Inventory | | |
|---|---|---|---|---|---|---|
| Material name | weight | Feed ratio | | rating | | mmol |
| E18b-2 | 433.5 | 2.5 | eq | 2.17 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E18b-2, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 750 mg of colorless oil was obtained after purification by column chromatography.

¹H NMR (600 MHz, CDCl₃) δ:4.86 (p, J=6.3 Hz, 2H), 3.62 (t, J=4.9 Hz, 4H), 2.68-2.60 (m, 8H), 2.55 (t, J=8.0 Hz, 4H), 2.27 (t, J=7.5 Hz, 4H), 1.61 (t, J=7.4 Hz, 4H), 1.49 (p, J=7.7, 6.7 Hz, 12H), 1.35-1.21 (m, 54H), 0.87 (t, J=7.0 Hz, 12H).

### Preparation Example 16 Preparation of Compound LQ104-E18b-2R

### Preparation of E18b-2R

### Reaction Formula:

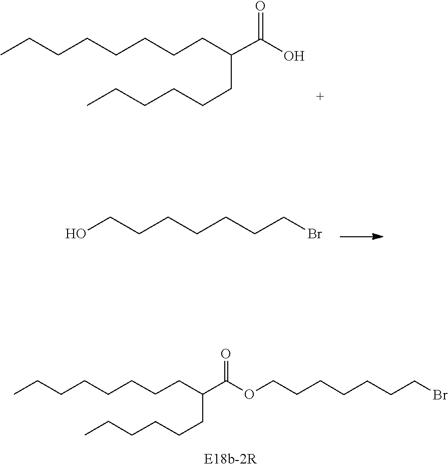 [SMILES: CCCCCCCCC(CCCCCC)C(=O)O ; OCCCCCCCBr ; CCCCCCCCC(CCCCCC)C(=O)OCCCCCCCBr]

### Material Ratio:

| | Molecular | | | Inventory | | |
|---|---|---|---|---|---|---|
| Material name | weight | Feed ratio | | rating | | mmol |
| 7-Bromo-1-heptanol | 195 | 1 | eq | 9.75 | g | 50 |
| 2-Hexyldecanoic acid | 256.43 | 1.1 | eq | 14.1 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

2-Hexyldecanoic acid, DCC, DMAP, and DCM were added to a 1 L reaction flask, followed by 7-bromo-1-heptanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 14.1 g of colorless oil was obtained after purification by column chromatography.

### Preparation of LQ104-E18b-2R

### Reaction Formula:

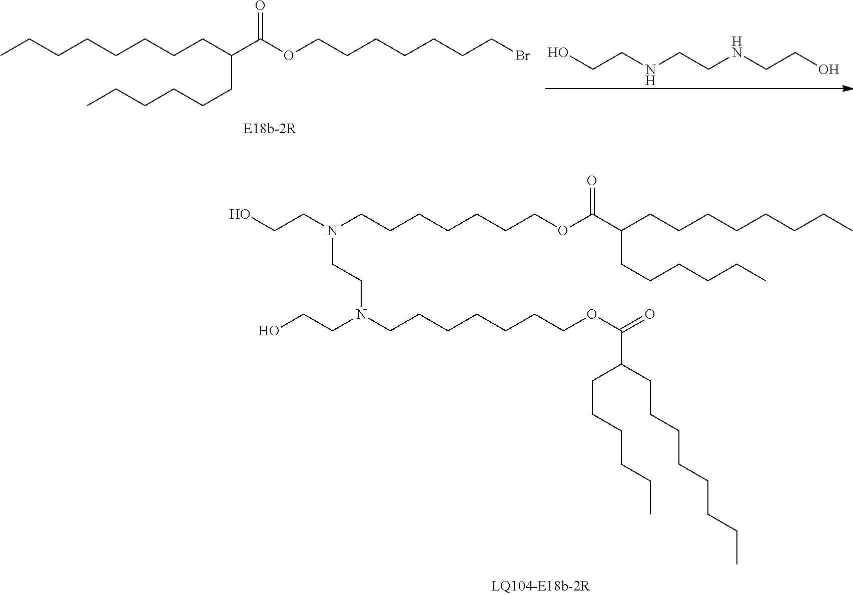

### Material Ratio:

| | Molecular | | | Inventory | | |
|---|---|---|---|---|---|---|
| Material name | weight | Feed ratio | | rating | | mmol |
| E18b-2R | 433.5 | 2.5 | eq | 2.17 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E18b-2R, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 690 mg of colorless oil was obtained after purification by column chromatography.

¹H NMR (600 MHz, CDCl₃) δ:4.05 (t, J=6.7 Hz, 4H), 3.77 (t, J=4.8 Hz, 4H), 2.96-2.87 (m, 8H), 2.81 (t, J=8.2 Hz, 4H), 2.30 (tt, J=8.9, 5.3 Hz, 2H), 1.65-1.52 (m, 12H), 1.46-1.38 (m, 4H), 1.37-1.19 (m, 54H), 0.87 (td, J=7.1, 1.5 Hz, 12H).

### Preparation Example 17 Preparation of Compound LQ104-E18b-3

### Preparation of E18b-3

### Reaction Formula:

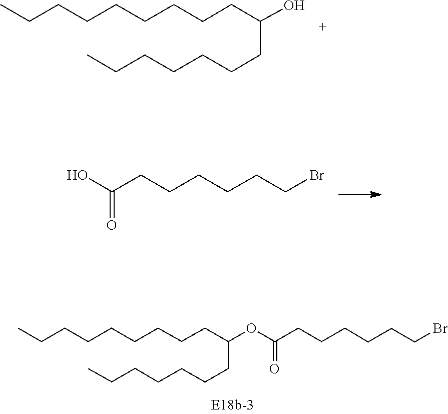 [SMILES: CCCCCCCCCC(O)CCCCCCC ; O=C(O)CCCCCCBr ; CCCCCCCCCC(CCCCCCC)OC(=O)CCCCCCBr]

### Material Ratio:

| | Molecular | | | Inventory | | |
|---|---|---|---|---|---|---|
| Material name | weight | Feed ratio | | rating | | mmol |
| 8-Heptadecanol | 256.47 | 1 | eq | 12.8 | g | 50 |
| 7-Bromoheptanoic acid | 209 | 1.1 | eq | 11.5 | g | 55 |
| DCC | 206 | 2 | eq | 20.6 | g | 100 |
| DMAP | 122 | 0.1 | eq | 610 | mg | 5 |
| DCM | | | | 300 | ml | |

### Operation Process:

7-Bromoheptanoic acid, DCC, DMAP, and DCM were added to a 1 L reaction flask, followed by 8-heptadecanol. After addition, stirring was performed at a room temperature for 12 hours. TLC (PE:EA=20:1) showed complete reaction (a product rf value was 0.6).

### Postprocessing:

A reaction solution was filtered and then subjected to rotary drying, and 13.7 g of colorless oil was obtained after purification by column chromatography.

### Preparation of LQ104-E18b-3

### Reaction Formula:

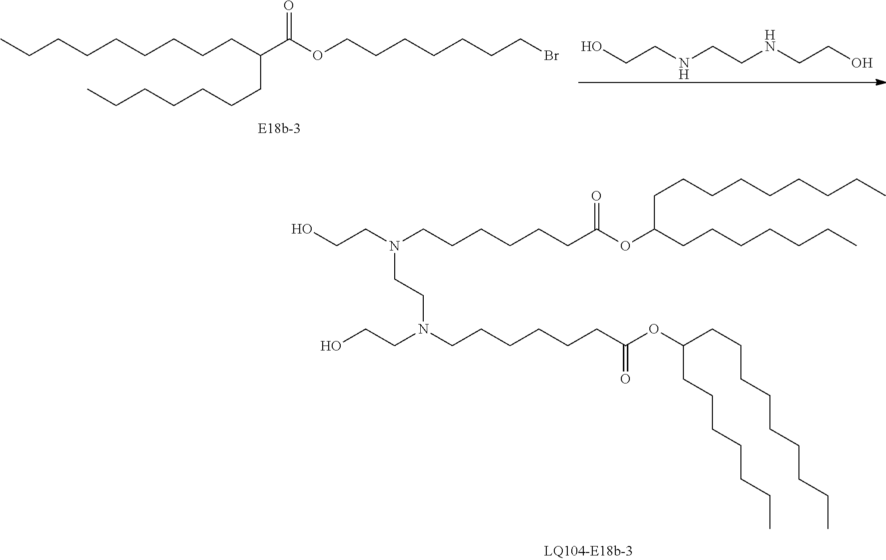 [SMILES: CCCCCCCCCC(CCCCCCC)C(=O)OCCCCCCCBr ; OCCNCCNCCO ; CCCCCCCCCC(CCCCCCC)OC(=O)CCCCCCN(CCO)CCN(CCO)CCCCCCC(=O)OC(CCCCCCC)CCCCCCCCC]

### Material Ratio:

| | Molecular | | | Inventory | | |
|---|---|---|---|---|---|---|
| Material name | weight | Feed ratio | | rating | | mmol |
| E18b-3 | 447.5 | 2.5 | eq | 2.24 | g | 5 |
| N,N′-bis(2- | 148 | 1 | eq | 300 | mg | 2 |
| hydroxyethyl)ethylenediamine | | | | | | |
| K₂CO₃ | 138 | 4 | eq | 1.1 | g | 8 |
| KI | 166 | 2 | eq | 664 | mg | 4 |
| Acetonitrile | | | | 40 | ml | |

### Operation Process:

E18b-3, N,N′-bis(2-hydroxyethyl)ethylenediamine, K₂CO₃, KI, and acetonitrile were added to a reaction flask, heated to 80° C., and stirred for 12 hours. TLC (DCM:MeOH=10:1) showed complete reaction (a product rf value was 0.5).

### Postprocessing:

A reaction solution was filtered and then subjected to rotary drying, and 790 mg of colorless oil was obtained after purification by column chromatography.

¹H NMR (600 MHz, CDCl₃) δ:4.86 (p, J=6.2 Hz, 2H), 3.61 (t, J=4.8 Hz, 4H), 2.67-2.60 (m, 8H), 2.54 (t, J=8.0 Hz, 4H), 2.27 (t, J=7.5 Hz, 4H), 1.62 (p, J=7.5 Hz, 4H), 1.49 (tt, J=7.6, 4.6 Hz, 12H), 1.37-1.20 (m, 58H), 0.87 (t, J=6.9 Hz, 12H).

### Preparation Example 18 Preparation of Compound LQ104-H3

A preparation route of LQ104-H3 is:

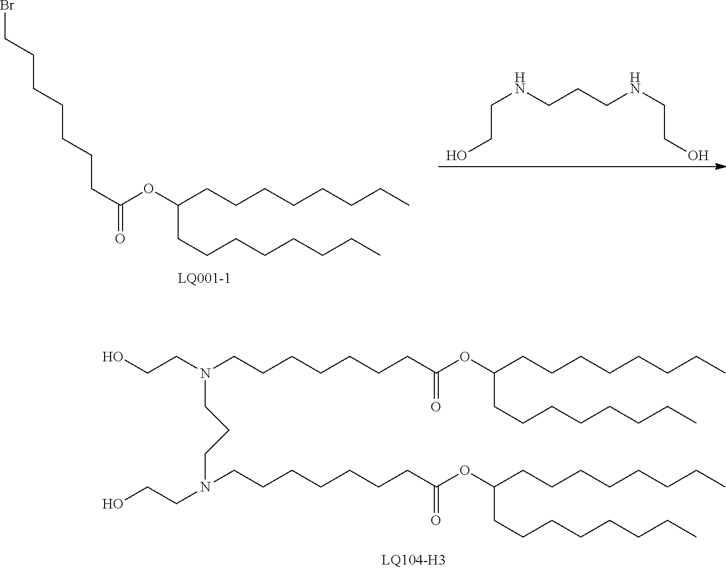 [SMILES: CCCCCCCCC(CCCCCCCC)OC(=O)CCCCCCCBr ; OCCNCCCNCCO ; CCCCCCCCC(CCCCCCCC)OC(=O)CCCCCCCN(CCO)CCCN(CCO)CCCCCCCC(=O)OC(CCCCCCCC)CCCCCCCC]

### Material Ratio:

| | Molecular | Inventory | | |
|---|---|---|---|---|
| Material name | weight | rating | | Mmol |
| LQ001-1 | 461.5 | 2 | g | 4.3 |
| N,N′-bis(2-hydroxyethyl)-1,3- | 235.15 | 510 | mg | 2.15 |
| propanediamine | | | | |
| K₂CO₃ | 138 | 2.1 | g | 15 |
| KI | 166 | 860 | mg | 5.2 |
| Acetonitrile | — | 30 | ml | — |

### Operation Process:

LQ001-1, N,N′-bis(2-hydroxyethyl)-1,3-propanediamine, K₂CO₃, KI, and acetonitrile were added to a 1 L reaction flask, heated to 80° C., and stirred for 12 hours. TLC (DCM:MeOH=10:1) showed complete reaction.

### Postprocessing:

A reaction solution was filtered and then subjected to rotary drying, and 850 mg of colorless oil, namely the compound LQ104-H3, was obtained after purification by column chromatography, with a yield of 43%.

### Mass Spectrometry Analysis:

There are strong ion peaks at m/z 924 and 925 in an ESI-MS positive ion mass spectrogram, which are consistent with a molecular weight of 923.5 of the compound.

¹HNMR (400 MHz, CDCl₃) δ:4.86 (p, 2H), 3.65-3.59 (m, 4H), 2.68-2.59 (m, 8H), 2.57-2.49 (m, 4H), 2.27 (t, 4H), 1.61(p, 7H), 1.49(dt, 12H), 1.28 (d, 61H), 0.87 (t, 12H).

Sources of the reagents used in the aforementioned preparation examples of the present application are as follows:
LQ001-1: self-made;
N,N′-bis(2-hydroxyethyl)ethylenediamine: purchased from Adamas Reagent Co., Ltd., with an item number: 013455310, and purity: RG, 98%;
K₂CO₃: purchased from Shanghai Rhawn Chemical Technology Co., Ltd., with an item number: RH425011, and purity: AR, 99%;
KI: purchased from Shanghai Rhawn Chemical Technology Co., Ltd., with an item number: RH432132, and purity: AR, 99%;
acetonitrile: purchased from Shanghai Titan Technology Co., Ltd., with an item number: 01111797, and purity: AR, ≥99.0%;
methyl tert-butyl ether: purchased from Shanghai Titan Technology Co., Ltd., with an item number: 01030342, and purity: AR, ≥99.0%;
propanedioic acid: purchased from Adamas Reagent Co., Ltd., with an item number: 01022573, and purity: RG, 99%;
dicyclohexylcarbodiimide (DCC): purchased from Adamas Reagent Co., Ltd., with an item number: 012041444, and purity: RG, 99%;
4-dimethylaminopyridine (DMAP): purchased from Adamas Reagent Co., Ltd., with an item number: 01271081, and purity: RG, 99%;
dichloromethane (DCM): purchased from Shanghai Titan Technology Co., Ltd., with an item number: 01111853, and purity: AR, ≥99.5%;
diatomaceous earth: purchased from Shanghai Titan Technology Co., Ltd., with an item number: 01589000, and purity: extra premium grade, ≥89.0%, 200 mesh;
8-pentadecanol: self-made;
6-bromohexanoic acid: purchased from Adamas Reagent Co., Ltd., with an item number: 01073739, and purity: RG, 98%+;
N,N′-bis(2-hydroxyethyl)ethylenediamine: purchased from Adamas Reagent Co., Ltd., with an item number: 013455310, and purity: RG, 98%;
9-heptadecanol: purchased from Dalian Ruiying Technology Co., Ltd., with purity: 98%;
5-bromovaleric acid: purchased from Bide Pharmatech, with an item number: BD9634, and purity: 98%;
10-nonadecanol: self-made;
4-bromobutyric acid: purchased from Shanghai Titan Technology Co., Ltd., with an item number: 011016520, and purity: RG, 99%+;
7-bromoheptanoic acid: purchased from Shanghai Titan Technology Co., Ltd., with an item number: 012345536, and purity: RG, 98%;
7-pentadecanol: self-made;
5-bromopentan-1-ol: purchased from Bide Pharmatech, with purity: 98%;
2-hexyldecanoic acid: purchased from Bide Pharmatech, with an item number: BD75392, and purity: 98%;
8-bromooctanoic acid: purchased from Jiangsu Aikon, with purity: 98%;
6-bromo-1-hexanol: purchased from Adamas Reagent Co., Ltd., with an item number: 01074359, and purity: RG, 98%;
8-heptadecanol: self-made;
7-bromo-1-heptanol: purchased from Adamas Reagent Co., Ltd., with an item number: 01001821, and purity: RG, 98%; and
N,N′-bis(2-hydroxyethyl)-1,3-propanediamine (purchased from Beijing YSI Chemical, with purity: 95%).

### Example 1

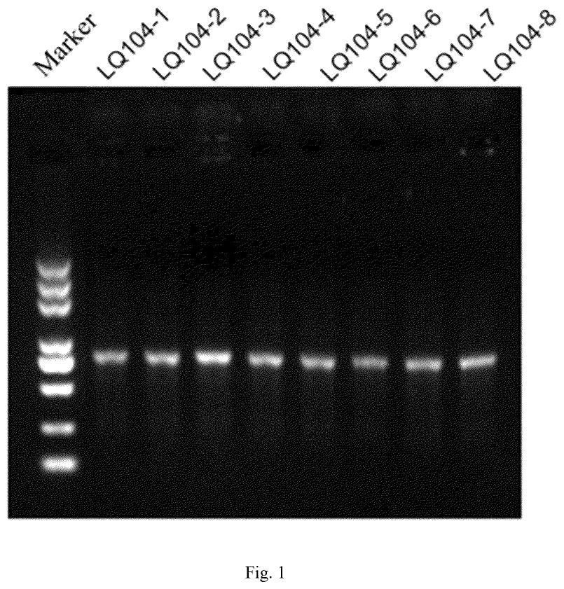

Example 1 is used to verify whether a lipid nanoparticle (LNP) preparation prepared from the ionizable lipid compound disclosed in the present application can effectively encapsulate mRNA and maintain structural integrity of the mRNA. The ionizable lipid compounds finally prepared in Preparation Examples 1 and 2, distearoyl phosphatidylcholine (DSPC, purchased from NIPPON FINE CHEMICAL CO., LTD, with an item number: 501005), cholesterol (purchased from NIPPON FINE CHEMICAL CO., LTD, with an item number: 001001), and dimyristoyl glycerol-polyethylene glycol 2000 (DMG-PEG2000, purchased from Guobang Pharmaceutical Co., Ltd., with an item number: 002005) were respectively dissolved in an ethanol (manufacturer: Nanjing Chemical Reagent Co., Ltd., purity 99.6%) solution, and then mixed according to a certain molar ratio to prepare an ethanol solution mixed with lipids, with a total lipid concentration of 12.5 mM (the measurement unit “M” used in the present application refers to mol/L). Self-made firefly luciferase (Fluc) mRNA or self-made SARS-CoV-2 spike protein (Spike) mRNA (SARS-CoV-2 spike protein mRNA, see Tan, S. et al., bioRxiv 2022.05.10.491301.) was diluted in 50 mM of citrate buffer with pH of 4.0 to obtain an mRNA solution. By using a microfluidic device, a flow rate was controlled at 12 mL/min, a volume ratio of the ethanol solution mixed with the lipids to the mRNA solution prepared in the previous steps was controlled at 1:3, and a lipid nanoparticle was prepared with a nitrogen-to-phosphorus ratio of (3-15):1 between ionizable lipids and mRNA. Dialysis was performed with 0.01 M phosphate buffer saline (PBS) for 12 to 24 hours to remove ethanol. Finally, the LNP solution was filtered through a sterile filter with a pore size of 0.22 m (manufacturer: Millex, item number: SLGPR33RB) and concentrated by ultrafiltration (manufacturer: Amicon-Ultra, molecular weight cut-off: 10 KDa) to obtain the LNP preparation obtained by encapsulating Fluc mRNA or Spike mRNA with the ionizable lipid, the DSPC, the cholesterol, and the DMG-PEG2000 as described in the present application. Wherein, a molar ratio of the ionizable lipid compound to the DSPC, the cholesterol, and the DMG-PEG2000, as well as a nitrogen-to-phosphorus ratio of the ionizable lipid to mRNA, are shown in Table 1. A particle size and polymer dispersity index (PDI) of each LNP preparation were measured by using a dynamic light scattering method and a Malvern Zetasizer Ultra instrument (manufacturer: Malvern); an encapsulation efficiency of LNP was measured by using a Quant-it Ribogreen RNA quantitative assay kit (manufacturer: ThermoFisher Scientific, item number: R11490); and an integrity of mRNA was investigated by nucleic acid gel electrophoresis (electrophoresis apparatus, manufacturer: Shanghai Tanon), and test results were shown in Table 1 and FIG. 1. FIG. 1 is a nucleic acid gel electrophoresis diagram of each LNP preparation prepared in this example. Wherein, gel was 1% agarose gel (manufacturer: Biowest; item number: BY-R0100), and a test condition was to perform electrophoresis at 160 V for 20 minutes.

TABLE 1

| | | Nitrogen-to- | Particle | | |
|---|---|---|---|---|---|
| | Molar percentage content of | phosphorus | size | | Encapsulation |
| Group | preparation components (%) | ratio | (nm) | PDI | efficiency (%) |
| LQ104- | LQ104:DSPC:cholesterol:DMG- | 6:1 | 86.00 | 0.105 | 94.1 |
| 1 | PEG = 43:11:44.4:1.6 | | | | |
| LQ104- | LQ104:DSPC:cholesterol:DMG- | 8:1 | 74.36 | 0.076 | 95.5 |
| 2 | PEG = 43:11:44.4:1.6 | | | | |
| LQ104- | LQ104:DSPC:cholesterol:DMG- | 6:1 | 89.01 | 0.111 | 94.6 |
| 3 | PEG = 40:11:47.4:1.6 | | | | |
| LQ104- | LQ104:DSPC:cholesterol:DMG- | 8:1 | 93.54 | 0.109 | 95.7 |
| 4 | PEG = 40:11:47.4:1.6 | | | | |
| LQ104- | LQ104:DSPC:cholesterol:DMG- | 6:1 | 101.97 | 0.058 | 95.6 |
| 5 | PEG = 40:12:46.4:1.6 | | | | |
| LQ104- | LQ104:DSPC:cholesterol:DMG- | 8:1 | 94.19 | 0.105 | 94.9 |
| 6 | PEG = 40:12:46.4:1.6 | | | | |
| LQ104- | LQ104:DSPC:cholesterol:DMG- | 6:1 | 117.37 | 0.062 | 95.1 |
| 7 | PEG = 47.4:10:41:1.6 | | | | |
| LQ104- | LQ104:DSPC:cholesterol:DMG- | 8:1 | 93.07 | 0.061 | 94.2 |
| 8 | PEG = 47.4:10:41:1.6 | | | | |
| LQ104- | LQ104:DSPC:cholesterol:DMG- | 6:1 | 93.49 | 0.029 | 92.3 |
| 9 | PEG = 50:10:38.5:1.5 | | | | |
| LQ104- | LQ104:DSPC:cholesterol:DMG- | 12:1 | 92.89 | 0.092 | 93.4 |
| 10 | PEG = 50:10:38.5:1.5 | | | | |
| LQ107 | LQ107:DSPC:cholesterol:DMG- | 6:1 | 83.56 | 0.025 | 83.7 |
| | PEG = 50:10:38.5:1.5 | | | | |

In the art, if the PDI was less than 0.3, it indicated that the size of the nanoparticles in the LNP preparation was relatively uniform; the encapsulation efficiency was used to indicate whether the LNP can effectively encapsulate mRNA, and if the encapsulation efficiency was higher than 7000, it indicated that the LNP can effectively encapsulate the mRNA; and a single and bright band in the agarose gel electrophoresis diagram can indicate mRNA structural integrity. Wherein, the more the PDI tends towards 0, the better, and the more the encapsulation efficiency tends towards 100%, the better. It can be seen from Table 1 and FIG. 1 that the particle size of each LNP in the present application ranges from 70 nm to 120 nm, with the PDI of less than 0.3 and the encapsulation efficiency of above 80%. Specifically, the encapsulation efficiency of the LQ104 series remained stable at above 90%, while the encapsulation efficiency of the LNP preparation made from LQ107 was 83.7%. It can be seen that the LNP preparations prepared according to the molar ratio and the nitrogen-to-phosphorus ratio shown in Table 1 can effectively encapsulate mRNA and maintain mRNA structural integrity. Moreover, it can also be seen from Table 1 and experimental data not exhaustively listed in the present application that performance of the LQ104 series was better than that of the LQ107 series. In addition, according to the experimental data verified but not exhaustively listed in the present application, regardless of the mRNA encapsulated, even if there are slight differences in the data obtained from the above in vitro experiments, it does not affect the above conclusion.

### Example 2

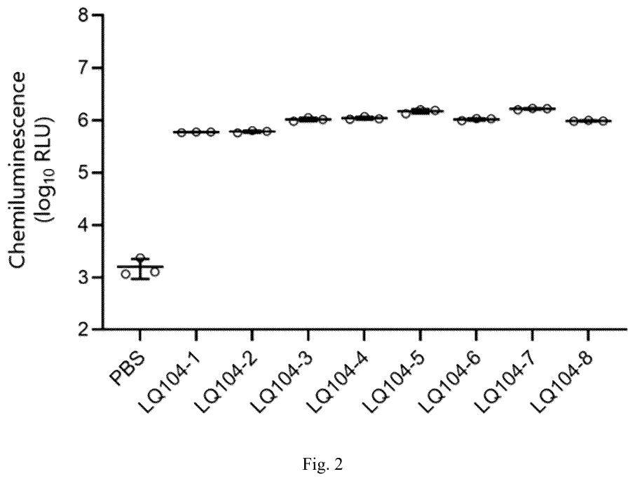

In this example, we validated in vitro cell delivery and expression of the LNP preparation of the present application through in vitro cell experiments. 10000 293FT cells per well were planted in a 96-well plate and cultured overnight until the cells adhere to the wall. The LNP preparations LQ104-1 to LQ104-8 from Example 1 were added to a cell culture fluid of the 96-well plate, with an mRNA content of 100 nanograms (ng) per well. Before adding the LNP preparation, the cell culture medium was replaced with an antibiotic-free DMEM culture fluid containing 10% fetal bovine serum (manufacturer: Gibco, item number: C11995500BT), and continued to culture for 24 hours, then the cell culture fluid was discarded, and a cell lysis solution containing D-fluorescein potassium salt (manufacturer: PerkinElmer, item number: 122799, final concentration 1 mM) and ATP (manufacturer: ApexBio, item number: C6931, final concentration 2 mM) was used to lyse cells at a dose of 100 μL/well. An enzyme-linked immunosorbent assay (manufacturer: thermo scientific) was used to detect chemiluminescence intensity. Test results were shown in FIG. 2. In FIG. 2, PBS serves as a negative control, and chemiluminescence intensity readings corresponding to this group may be considered as background readings. The higher the chemiluminescence intensity reading, the higher the expression. It can be seen from FIG. 2 that compared with the PBS group, the readings of LQ104-1 to LQ104-8 groups increased significantly, indicating that all LNPs can effectively deliver Fluc mRNA into cells and achieve its expression.

### Example 3

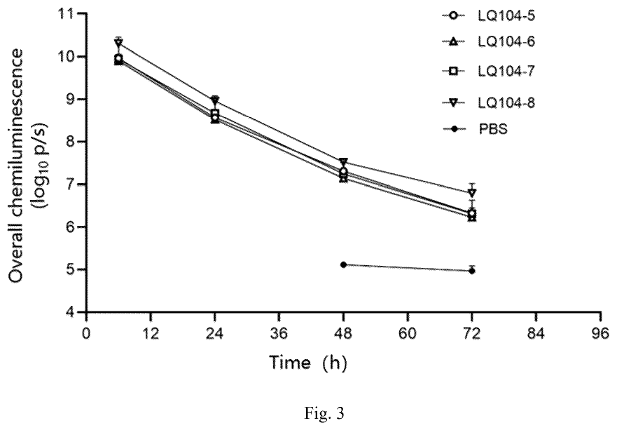

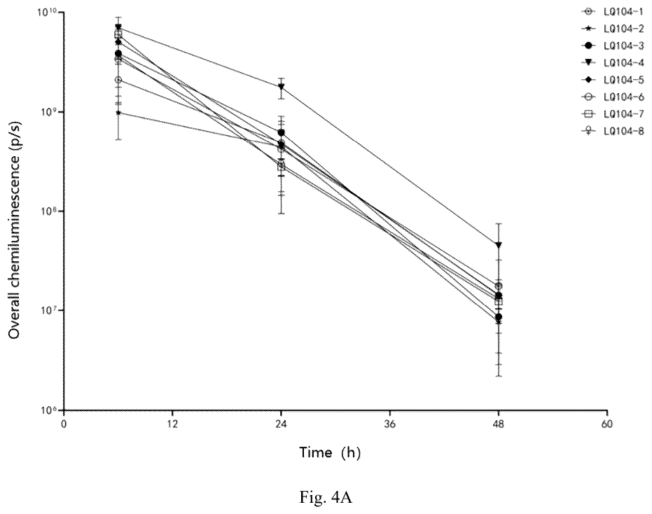

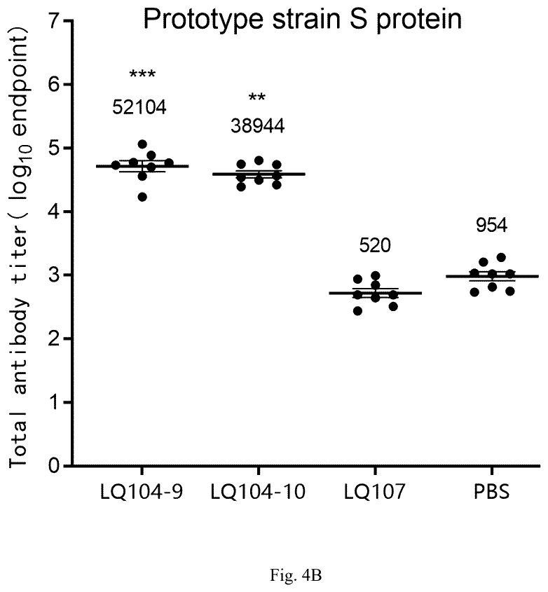

In this example, LQ104-1 to LQ104-8 (encapsulating Fluc mRNA) from Example 1 were injected via tail vein into female Balb/C mice (Viton Lihua) aged 6 to 8 weeks at a dose of 5 μg/mouse (n=3, i.e. 3 mice were used for injection and testing in each group, and the data results presented were the mean measurements of each group). D-fluorescein potassium salt was intraperitoneally injected at specific time points after administration (in this example, 6th hour, 24th hour, and 48th hour), and then the luminescence was detected by an IVIS Spectrum small animal living imager instrument (manufacturer: PerkinElmer). The overall luminescence intensity of live expression sites (such as the liver) in mice was counted. The higher the luminescence intensity, the higher the expression of luciferase, indicating that the corresponding LNP preparation was better expressed in mice. The overall luminescence intensity was the luminescence intensity data, measured by bioluminescence imaging, of the luminescent site obtained 6 to 15 minutes (min) after intraperitoneal injection of D-fluorescein potassium salt. The overall luminescence intensity of the in vivo expression area was counted by using Living Image software (manufacturer: PerkinElmer), furthermore, an area under the curve (AUC, unit: p/s*hour) was calculated by using GraphPad software, and the AUC in each example of the present application is the area under the curve of a line connecting measurement points of the overall luminescence intensity from the 4th or 6th hour after drug administration (using the experimental method of the present application, peak values are the same from 3-6 hours after drug administration) to the 48th hour after drug administration. Test results were shown in FIG. 3 and FIG. 4A, as well as Table 2.

TABLE 2
(AUC data in FIG. 4A)

| Group | AUC (p/s*hour) |
|---|---|
| LQ104-1 | 2.95E+10 |
| LQ104-2 | 1.83E+10 |
| LQ104-3 | 4.81E+10 |
| LQ104-4 | 1.01E+11 |
| LQ104-5 | 5.58E+10 |
| LQ104-6 | 3.98E+10 |
| LQ104-7 | 5.97E+10 |
| LQ104-8 | 3.96E+10 |

Note:
E+10 is the representation of scientific notation, representing 10 to the power of 10. For example, E+11 represents 10 to the power of 11, the same applies below.

Under normal circumstances, the order of magnitude of the readings of the overall luminescence intensity detected by the living imager in mice without administration treatment is 10⁵. It can be seen from FIG. 3 and FIG. 4A, as well as Table 2 that LQ104-1 to LQ104-8 were strongly expressed in mice. Furthermore, in various examples of the present application, we determined the LNP preparation with better expression by observing the area under the curve (AUC) of the line connecting the measurement points. The larger the area under the curve, the better the expression effect.

LQ104-9, LQ104-10, and LQ107 (encapsulating Spike mRNA) were injected intramuscularly into female Balb/C mice aged 6 to 8 weeks at a dose of 2 μg/mouse, and the same LNP preparation was repeatedly injected on the 21st day of the first injection. Whole blood was collected from mice at a specific time point after the second administration (the data in this example is on the 7th day after the second administration). The collected blood was centrifuged at 4° C. and 2000×g for 10 minutes to separate the serum from the whole blood; subsequently, the serum was inactivated in a water bath at 56° C. for 30 minutes and stored at −80° C. for analysis. A total antibody titer in serum was measured by an enzyme-linked immunosorbent assay (ELISA) method. Specifically, SARS-CoV-2 (2019-nCoV) Spike S1+S2 ECD-His Recombinant Protein (manufacturer: Sinobiological, item number: 40589-V08B1) was used to coat the antigen, SARS-CoV-2 (2019-nCoV) Spike Neutralizing Antibody Mouse Mab (manufacturer: Sinobiological, item number: 40591-MM43) was used as a control, 2% bovine serum albumin (BSA) was blocked, Peroxidase AffiniPure Goat Anti-Mouse IgG (H+L) (Jackson ImmunoResearch, item number: 115-035-003) secondary antibody was incubated, and TMB (Invitrogen, item number: 00-4201-56) developing was used according to the instructions to perform enzyme-linked immunosorbent assay (ELISA) analysis, so as to measure the Spike antibody titer (this data is the total antibody titer in mouse serum measured on the 7th day after the second administration, n=8). The test results were shown in FIG. 4.

It can be seen from FIG. 4 that compared with animals injected with PBS, the total Spike protein antibody titers in animals injected with LQ104-9 and LQ104-10 were significantly increased (statistically analyzed by ANOVA, **p<0.01, ***p<0.001, compared with the animal group injected with PBS), indicating effective expression of mRNA delivered by the vector after administration and induction of immune response. The total antibody titer of the animal group injected with LQ107 did not increase.

### Example 4

In this example, the ionizable lipids prepared from Preparation Example 3 to Preparation Example 17 were selected. Following the same method as in Example 1, an LNP preparation (encapsulating Fluc mRNA) was prepared according to a molar ratio and a nitrogen-to-phosphorus ratio shown in Table 3. Malvern Zetasizer Ultra was used to measure a particle size, a PDI, and a surface potential of each LNP preparation; a Quant-it Ribogreen RNA quantification assay kit (manufacturer: ThermoFisher Scientific, item number: R11490) was used to measure encapsulation efficiency of LNP; and a dye binding test of 6-(p-Toluidino)-2-naphthalene sulfonic acid sodium salt (TNS, purchased from Nanjing Xize Pharmaceutical Technology Co., Ltd., item number: XZ0743) was used to measure pKa of the LNP.

TABLE 3

| | Molar percentage | | | | | | |
|---|---|---|---|---|---|---|---|
| | content of | Nitrogen | Finished product feature | | | | |
| Selected | preparation | -to- | Particle | | Encapsulation | Surface | |
| cationic | components | phosphorus | size | | efficiency | potential | |
| lipid | (%) | ratio | (nm) | PDI | (%) | (mV) | pKa |
| LQ104- | LQ104-E15b- | 8:1 | 71.08 | 0.059 | 98.1 | −4.22 | 6.754 |
| E15b-1 | 1:DSPC:cholesterol:DMG- | | | | | | |
| | PEG = | | | | | | |
| | 40:11:47.4:1.6 | | | | | | |
| LQ104- | LQ104-E15b- | 8:1 | 56.47 | 0.059 | 96.8 | −6.81 | 6.470 |
| E15b-2 | 2:DSPC:cholesterol:DMG- | | | | | | |
| | PEG = | | | | | | |
| | 40:11:47.4:1.6 | | | | | | |
| LQ104- | LQ104-E15b- | 8:1 | 52.80 | 0.083 | 96.0 | −10.70 | 6.161 |
| E15b-3 | 3:DSPC:cholesterol:DMG- | | | | | | |
| | PEG = | | | | | | |
| | 40:11:47.4:1.6 | | | | | | |
| LQ104- | LQ104-E16b- | 8:1 | 84.84 | 0.044 | 97.2 | −3.35 | 7.109 |
| E16b-1 | 1:DSPC:cholesterol:DMG- | | | | | | |
| | PEG- | | | | | | |
| | 40:11:47.4:1.6 | | | | | | |
| LQ104- | LQ104-E16b- | 8:1 | 75.44 | 0.098 | 97.9 | −4.38 | 6.761 |
| E16b-2 | 2:DSPC:cholesterol:DMG- | | | | | | |
| | PEG = | | | | | | |
| | 40:11:47.4:1.6 | | | | | | |
| LQ104- | LQ104-E16b- | 8:1 | 78.35 | 0.091 | 97.6 | −5.94 | 6.773 |
| E16b-3 | 3:DSPC:cholesterol:DMG- | | | | | | |
| | PEG = | | | | | | |
| | 40:11:47.4:1.6 | | | | | | |
| LQ104- | LQ104-E16b- | 8:1 | 89.68 | 0.088 | 96.8 | −4.59 | 6.802 |
| E16b- | 3R:DSPC:cholesterol:DMG- | | | | | | |
| 3R | PEG = | | | | | | |
| | 40:11:47.4:1.6 | | | | | | |
| LQ104- | LQ104-E17b- | 8:1 | 85.67 | 0.082 | 96.9 | −1.066 | 7.19 |
| E17b-1 | 1:DSPC:cholesterol:DMG- | | | | | | |
| | PEG = | | | | | | |
| | 40:11:47.4:1.6 | | | | | | |
| LQ104- | LQ104-E17b- | 8:1 | 74.14 | 0.045 | 97.4 | −1.852 | 6.852 |
| E17b-2 | 2:DSPC:cholesterol:DMG- | | | | | | |
| | PEG = | | | | | | |
| | 40:11:47.4:1.6 | | | | | | |
| LQ104- | LQ104-E17b- | 8:1 | 85.57 | 0.058 | 97.4 | −2.788 | 7.15 |
| E17b-3 | 3:DSPC:cholesterol:DMG- | | | | | | |
| | PEG = | | | | | | |
| | 40:11:47.4:1.6 | | | | | | |
| LQ104- | LQ104-E17b- | 8:1 | 77.34 | 0.087 | 97.4 | −2.203 | 7.153 |
| E17b- | 3R:DSPC:cholesterol:DMG- | | | | | | |
| 3R | PEG = | | | | | | |
| | 40:11:47.4:1.6 | | | | | | |
| LQ104- | LQ104-E17b- | 8:1 | 72.30 | 0.061 | 98.0 | −3.406 | 6.469 |
| E17b-4 | 4:DSPC:cholesterol:DMG- | | | | | | |
| | PEG = | | | | | | |
| | 40:11:47.4:1.6 | | | | | | |
| LQ104- | LQ104-E18b- | 8:1 | 84.46 | 0.084 | 97.5 | −1.109 | 7.262 |
| E18b-2 | 2:DSPC:cholesterol:DMG- | | | | | | |
| | PEG = | | | | | | |
| | 40:11:47.4:1.6 | | | | | | |
| LQ104- | LQ104-E18b- | 8:1 | 80.10 | 0.081 | 97.4 | −2.013 | 7.033 |
| E18b- | 2R:DSPC:cholesterol:DMG- | | | | | | |
| 2R | PEG = | | | | | | |
| | 40:11:47.4:1.6 | | | | | | |
| LQ104- | LQ104-E18b- | 8:1 | 90.39 | 0.063 | 97.4 | −2.466 | 7.039 |
| E18b-3 | 3:DSPC:cholesterol:DMG- | | | | | | |
| | PEG = | | | | | | |
| | 40:11:47.4:1.6 | | | | | | |

It can be seen from Table 3 that the particle sizes of the LNP preparations prepared from the ionizable lipids described in Preparation Example 3 to Preparation Example 17 were between 50 nm and 100 nm; PDI was less than 0.3, specifically between 0.044 and 0.098; and the encapsulation efficiency was higher than 9600. It indicated that the LNP preparation in this example can effectively encapsulate mRNA; and the surface potential was weakly negative, with a pKa between 6 and 7.3, which is equivalent to the recognized range in the art.

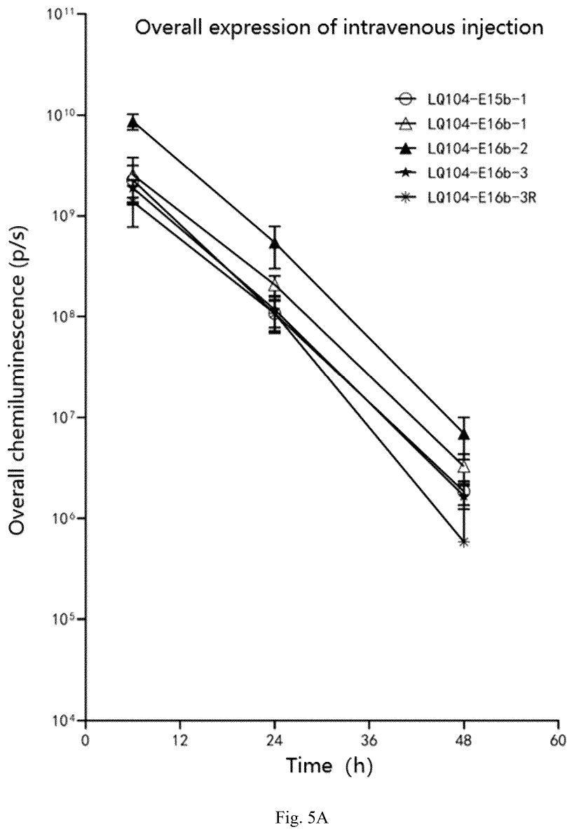

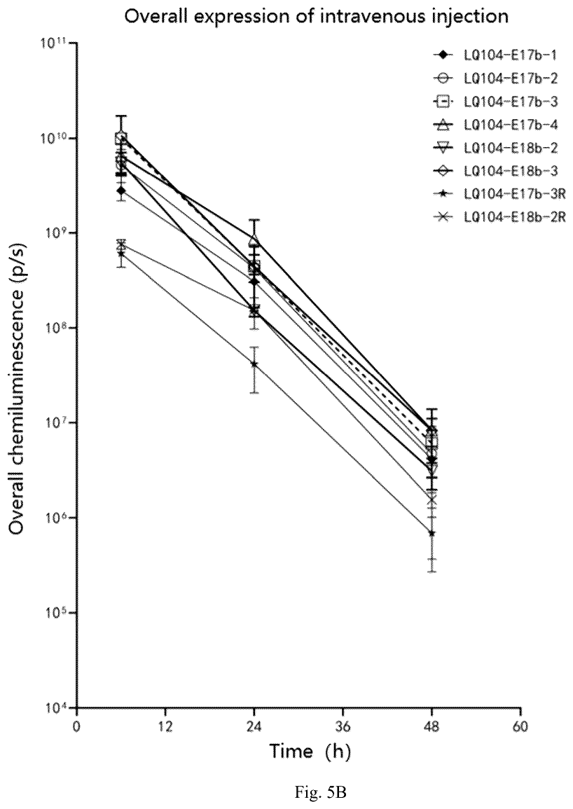

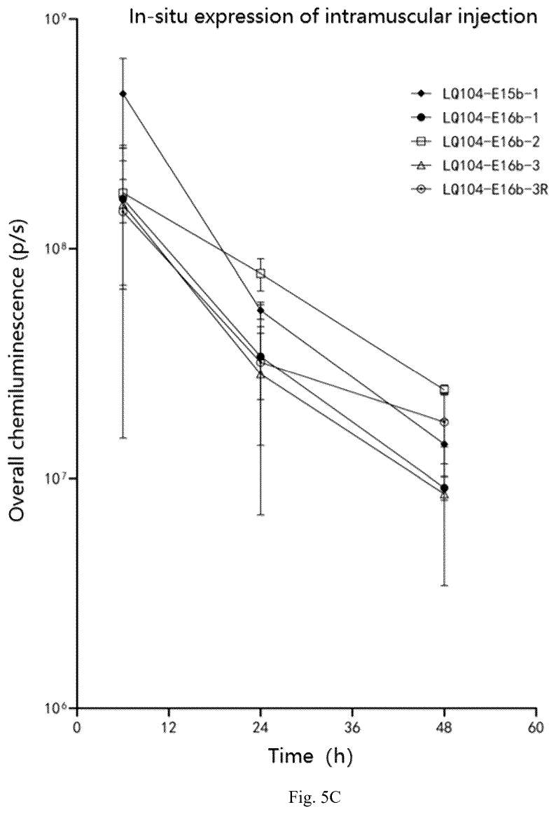

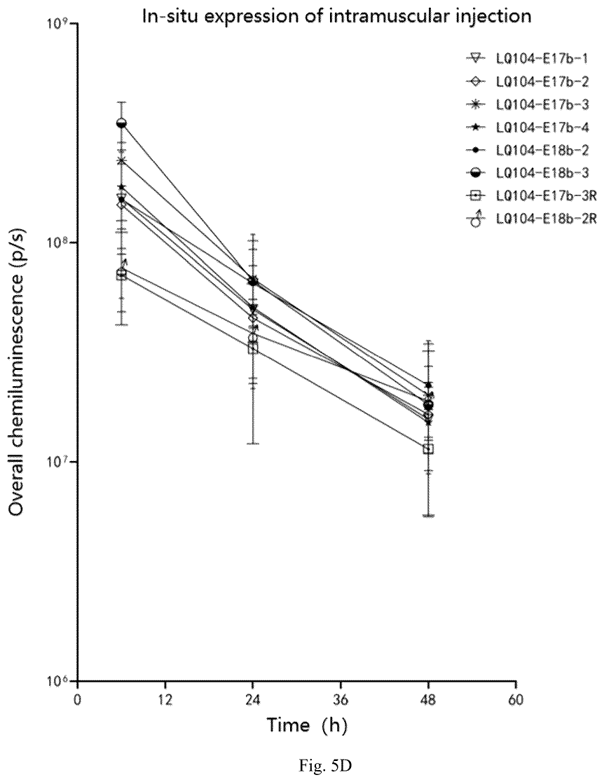

Furthermore, referring to the mouse in-vivo experimental method of Example 3, each group of LNP preparations in this example was injected into female Balb/C mice aged 6 to 8 weeks through tail vein injection or intramuscular injection of the lower limbs at a dose of 5 μg/mouse. The overall luminescence intensity of the liver or lower limb administration site was counted, and the test results were shown in FIG. 5A to FIG. 5D and Table 4A to Table 4D). Wherein, FIG. 5A and FIG. 5B showed the luminescence statistical results of the liver sites in mice after intravenous injection administration, while FIG. 5C and FIG. 5D showed the luminescence statistical results of the lower limb administration site in mice after intramuscular injection administration. It can be seen from FIG. 5A to FIG. 5D and Table 4A to Table 4D that the LNP preparation tested in this example had strong expression in mice, which indicated that the LNP preparations corresponding to the ionizable lipids described in Preparation Example 3 to Preparation Example 17 can effectively deliver mRNA into the body and express the same. We also tested other administration routes, such as intraperitoneal injection administration and subcutaneous injection administration, and the results showed that the LNP preparation in this example could be expressed under multiple administration routes.

TABLE 4A
(AUC data in FIG. 5A)

| Group | AUC (p/s*hour) |
|---|---|
| LQ104-E15b-1 | 2.26E+10 |
| LQ104-E16b-1 | 2.74E+10 |
| LQ104-E16b-2 | 8.98E+10 |
| LQ104-E16b-3 | 1.95E+10 |
| LQ104-E16b-3R | 1.47E+10 |

TABLE 4B
(AUC data in FIG. 5B)

| Group | AUC (p/s*hour) |
|---|---|
| LQ104-E17b-1 | 3.16E+10 |
| LQ104-E17b-2 | 5.58E+10 |
| LQ104-E17b-3 | 9.91E+10 |
| LQ104-E17b-4 | 7.68E+10 |
| LQ104-E18b-2 | 5.32E+10 |
| LQ104-E18b-3 | 1.06E+11 |
| LQ104-E17b-3R | 6.33E+09 |
| LQ104-E18b-2R | 1.01E+10 |

TABLE 4C
(AUC data in FIG. 5C)

| Group | AUC (p/s*hour) |
|---|---|
| LQ104-E15b-1 | 5.58E+09 |
| LQ104-E16b-1 | 2.30E+09 |
| LQ104-E16b-2 | 3.50E+09 |
| LQ104-E16b-3 | 2.10E+09 |
| LQ104-E16b-3R | 2.19E+09 |

TABLE 4D
(AUC data in FIG. 5D)

| Group | AUC (p/s*hour) |
|---|---|
| LQ104-E17b-1 | 2.66E+09 |
| LQ104-E17b-2 | 2.49E+09 |
| LQ104-E17b-3 | 3.82E+09 |
| LQ104-E17b-4 | 2.86E+09 |
| LQ104-E18b-2 | 3.06E+09 |
| LQ104-E18b-3 | 4.80E+09 |
| LQ104-E17b-3R | 1.47E+09 |
| LQ104-E18b-2R | 1.73E+09 |

### Example 5

In this example, LQ104-E16b-2 obtained from Preparation Example 7, LQ104-E17b-4 obtained from Preparation Example 14, and LQ104-EF8b-3 obtained from Preparation Example 17 were selected and divided into 8 groups each to prepare an LNP preparation (encapsulating Fluc mRNA) according to preparation components and a nitrogen-to-phosphorus ratio described in Table 5 below. Their particle sizes, PDI, and encapsulation efficiency were measured, and results were shown in Table 5.

TABLE 5

| | | Nitrogen- | Finished product feature | | |
|---|---|---|---|---|---|
| | Molar percentage content | to- | Particle | | Encapsulation |
| | of preparation components | phosphorus | size | | efficiency |
| Group | (%) | ratio | (nm) | PDI | (%) |
| LQ104-E16b-2 | LQ104-E16b- | 6:1 | 71.38 | 0.085 | 97.7 |
| (f1) | 2:DSPC:cholesterol:DMG- | | | | |
| | PEG = 43:11:44.4:1.6 | | | | |
| LQ104-E16b-2 | LQ104-E16b- | 8:1 | 68.50 | 0.102 | 97.6 |
| (f2) | 2:DSPC:cholesterol:DMG- | | | | |
| | PEG = 43:11:44.4:1.6 | | | | |
| LQ104-E16b-2 | LQ104-E16b- | 6:1 | 69.54 | 0.107 | 97.7 |
| (f3) | 2:DSPC:cholesterol:DMG- | | | | |
| | PEG = 40:11:47.4:1.6 | | | | |
| LQ104-E16b-2 | LQ104-E16b- | 8:1 | 70.06 | 0.111 | 97.9 |
| (f4) | 2:DSPC:cholesterol:DMG- | | | | |
| | PEG = 40:11:47.4:1.6 | | | | |
| LQ104-E16b-2 | LQ104-E16b- | 6:1 | 75.09 | 0.053 | 98.2 |
| (f5) | 2:DSPC:cholesterol:DMG- | | | | |
| | PEG = 40:12:46.4:1.6 | | | | |
| LQ104-E16b-2 | LQ104-E16b- | 8:1 | 69.48 | 0.101 | 98.1 |
| (f6) | 2:DSPC:cholesterol:DMG- | | | | |
| | PEG = 40:12:46.4:1.6 | | | | |
| LQ104-E16b-2 | LQ104-E16b- | 6:1 | 79.65 | 0.076 | 98.0 |
| (f7) | 2:DSPC:cholesterol:DMG- | | | | |
| | PEG = 47.4:10:41:1.6 | | | | |
| LQ104-E16b-2 | LQ104-E16b- | 8:1 | 75.65 | 0.090 | 97.5 |
| (f8) | 2:DSPC:cholesterol:DMG- | | | | |
| | PEG = 47.4:10:41:1.6 | | | | |
| LQ104-E18b-3 | LQ104-E18b- | 6:1 | 84.51 | 0.082 | 97.9 |
| (f1) | 3:DSPC:cholesterol:DMG- | | | | |
| | PEG = 43:11:44.4:1.6 | | | | |
| LQ104-E18b-3 | LQ104-E18b- | 8:1 | 79.65 | 0.048 | 97.7 |
| (f2) | 3:DSPC:cholesterol:DMG- | | | | |
| | PEG = 43:11:44.4:1.6 | | | | |
| LQ104-E18b-3 | LQ104-E18b- | 6:1 | 79.27 | 0.103 | 98.5 |
| (f3) | 3:DSPC:cholesterol:DMG- | | | | |
| | PEG = 40:11:47.4:1.6 | | | | |
| LQ104-E18b-3 | LQ104-E18b- | 8:1 | 75.10 | 0.076 | 98.0 |
| (f4) | 3:DSPC:cholesterol:DMG- | | | | |
| | PEG = 40:11:47.4:1.6 | | | | |
| LQ104-E18b-3 | LQ104-E18b- | 6:1 | 87.78 | 0.049 | 99.0 |
| (f7) | 3:DSPC:cholesterol:DMG- | | | | |
| | PEG = 47.4:10:41:1.6 | | | | |
| LQ104-E18b-3 | LQ104-E18b- | 8:1 | 89.61 | 0.047 | 99.0 |
| (f8) | 3:DSPC:cholesterol:DMG- | | | | |
| | PEG = 47.4:10:41:1.6 | | | | |
| LQ104-E17b-4 | LQ104-E17b- | 6:1 | 72.93 | 0.090 | 97.9 |
| (f1) | 4:DSPC:cholesterol:DMG- | | | | |
| | PEG = 43:11:44.4:1.6 | | | | |
| LQ104-E17b-4 | LQ104-E17b- | 8:1 | 68.86 | 0.073 | 97.2 |
| (f2) | 4:DSPC:cholesterol:DMG- | | | | |
| | PEG = 43:11:44.4:1.6 | | | | |
| LQ104-E17b-4 | LQ104-E17b- | 6:1 | 69.36 | 0.074 | 97.1 |
| (f3) | 4:DSPC:cholesterol:DMG- | | | | |
| | PEG = 40:11:47.4:1.6 | | | | |
| LQ104-E17b-4 | LQ104-E17b- | 8:1 | 66.66 | 0.076 | 97.9 |
| (f4) | 4:DSPC:cholesterol:DMG- | | | | |
| | PEG = 40:11:47.4:1.6 | | | | |
| LQ104-E17b-4 | LQ104-E17b- | 6:1 | 67.71 | 0.087 | 98.1 |
| (f5) | 4:DSPC:cholesterol:DMG- | | | | |
| | PEG = 40:12:46.4:1.6 | | | | |
| LQ104-E17b-4 | LQ104-E17b- | 8:1 | 65.15 | 0.058 | 98.6 |
| (f6) | 4:DSPC:cholesterol:DMG- | | | | |
| | PEG = 40:12:46.4:1.6 | | | | |
| LQ104-E17b-4 | LQ104-E17b- | 6:1 | 76.93 | 0.097 | 96.4 |
| (f7) | 4:DSPC:cholesterol:DMG- | | | | |
| | PEG = 47.4:10:41:1.6 | | | | |
| LQ104-E17b-4 | LQ104-E17b- | 8:1 | 68.00 | 0.097 | 97.9 |
| (f8) | 4:DSPC:cholesterol:DMG- | | | | |
| | PEG = 47.4:10:41:1.6 | | | | |

It can be seen from Table 5 that the particle size of the LNP preparation prepared from the compound LQ104-E16b-2, LQ104-E17b-4, or LQ104-E18b-3 according to the above components and nitrogen-to-phosphorus ratio was between 60 nm and 90 nm; PDI was all less than 0.3, and mostly concentrated below 0.1; and the encapsulation efficiency was above 9000, mainly concentrated between 9700 and 99%.

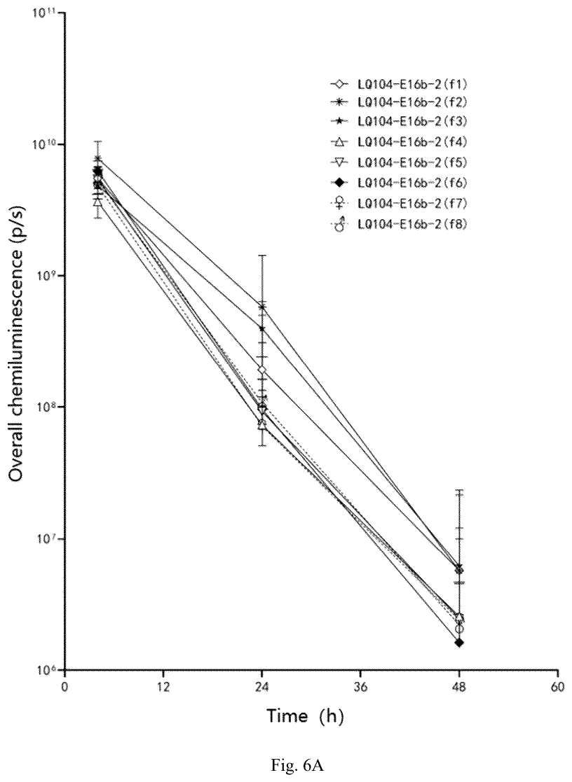

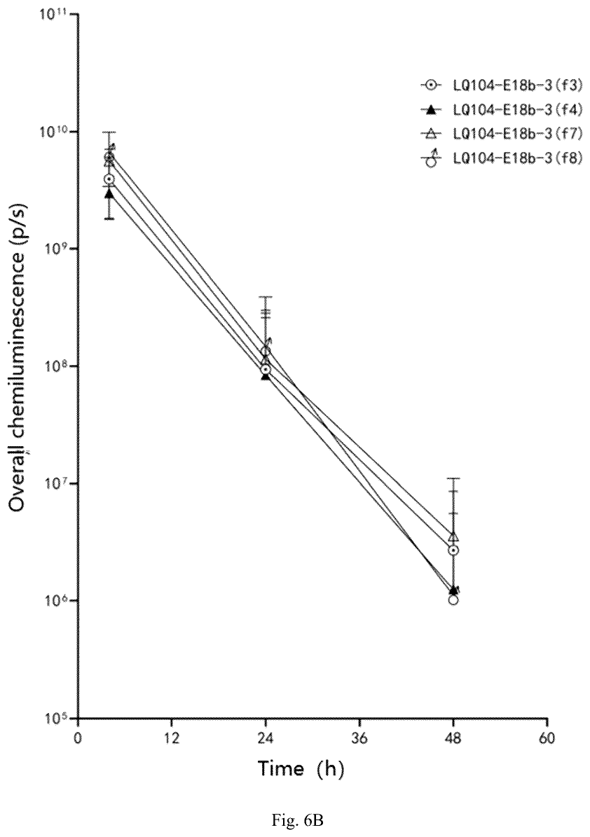

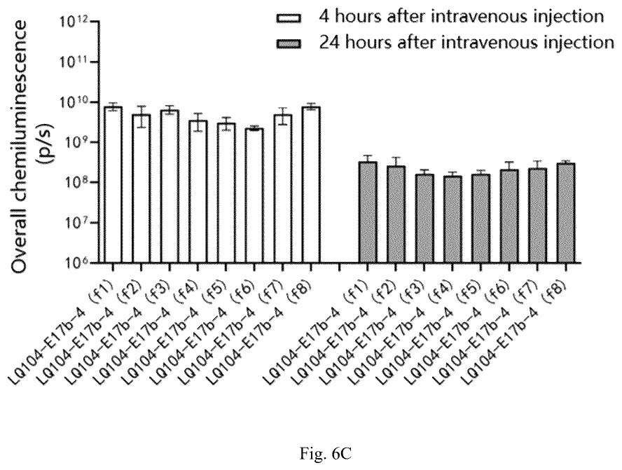

Similarly, referring to the mouse in-vivo experimental method of Example 3, all LNP preparations prepared in this example were injected into female Balb/C mice aged 6 to 8 weeks through the tail vein at a dose of 5 ag/mouse, and the overall luminescence intensity of the living liver sites of the mice was counted. Test results were shown in FIG. 6A to FIG. 6C and Table 6A to Table 6B. It can be seen that the LNP preparation in this example had strong expression in mice.

TABLE 6A
(AUC data in FIG. 6A)

| Group | AUC (p/s*hour) |
|---|---|
| LQ104-E16b-2(f1) | 5.93E+10 |
| LQ104-E16b-2(f2) | 9.13E+10 |
| LQ104-E16b-2(f3) | 5.73E+10 |
| LQ104-E16b-2(f4) | 3.82E+10 |
| LQ104-E16b-2(f5) | 5.67E+10 |
| LQ104-E16b-2(f6) | 6.49E+10 |
| LQ104-E16b-2(f7) | 5.02E+10 |
| LQ104-E16b-2(f8) | 5.54E+10 |

TABLE 6B
(AUC data in FIG. 6B)

| Group | AUC (p/s*hour) |
|---|---|
| LQ104-E18b-3(f3) | 4.14E+10 |
| LQ104-E18b-3(f4) | 3.17E+10 |
| LQ104-E18b-3(f7) | 5.92E+10 |
| LQ104-E18b-3(f8) | 7.00E+10 |

### Example 6

In this example, LQ104-E16b-2 obtained from Preparation Example 7 was selected as the ionizable lipid, 21 LNP preparations (encapsulating Fluo mRNA) were prepared in the same manner as Example 1 according to a molar ratio and a nitrogen-to-phosphorus ratio in Table 7, and their particle sizes, PDI, and encapsulation efficiency were measured. This example was mainly used to verify the optimal component content of DSPC and cholesterol in the LNP preparations. Wherein, based on the total mole number of the four components as 100%, the molar percentage content of LQ104-E16b-2 and DMG-PEG was kept unchanged, and performance of each LNP preparation prepared by changing the molar percentage content of DSPC and cholesterol was verified.

TABLE 7

| | | Nitrogen- | Finished product feature | | |
|---|---|---|---|---|---|
| | Molar percentage content | to- | Particle | | Encapsulation |
| | of preparation components | phosphorus | size | | efficiency |
| Group | (%) | ratio | (nm) | PDI | (%) |
| LQ104-E16b-2 | LQ104-E16b- | 8:1 | 90.12 | 0.139 | 97.1 |
| (DS-f1) | 2:DSPC:cholesterol:DMG- | | | | |
| | PEG = 40:0:58.4:1.6 | | | | |
| LQ104-E16b-2 | LQ104-E16b- | 8:1 | 82.52 | 0.081 | 96.8 |
| (DS-f2) | 2:DSPC:cholesterol:DMG- | | | | |
| | PEG = 40:2:56.4:1.6 | | | | |
| LQ104-E16b-2 | LQ104-E16b- | 8:1 | 73.31 | 0.073 | 97.3 |
| (DS-f3) | 2:DSPC:cholesterol:DMG- | | | | |
| | PEG = 40:4:54.4:1.6 | | | | |
| LQ104-E16b-2 | LQ104-E16b- | 8:1 | 70.79 | 0.101 | 97.3 |
| (DS-f4) | 2:DSPC:cholesterol:DMG- | | | | |
| | PEG = 40:6:52.4:1.6 | | | | |
| LQ104-E16b-2 | LQ104-E16b- | 8:1 | 66.62 | 0.100 | 97.1 |
| (DS-f5) | 2:DSPC:cholesterol:DMG- | | | | |
| | PEG = 40:8:50.4:1.6 | | | | |
| LQ104-E16b-2 | LQ104-E16b- | 8:1 | 68.99 | 0.092 | 96.9 |
| (DS-f6) | 2:DSPC:cholesterol:DMG- | | | | |
| | PEG = 40:10:48.4:1.6 | | | | |
| LQ104-E16b-2 | LQ104-E16b- | 8:1 | 66.01 | 0.063 | 97.2 |
| (DS-f7) | 2:DSPC:cholesterol:DMG- | | | | |
| | PEG = 40:12:46.4:1.6 | | | | |
| LQ104-E16b-2 | LQ104-E16b- | 8:1 | 63.79 | 0.072 | 97.0 |
| (DS-f8) | 2:DSPC:cholesterol:DMG- | | | | |
| | PEG = 40:14:44.4:1.6 | | | | |
| LQ104-E16b-2 | LQ104-E16b- | 8:1 | 69.85 | 0.089 | 97.0 |
| (DS-f9) | 2:DSPC:cholesterol:DMG- | | | | |
| | PEG = 40:16:42.4:1.6 | | | | |
| LQ104-E16b-2 | LQ104-E16b- | 8:1 | 64.29 | 0.103 | 97.2 |
| (DS-f10) | 2:DSPC:cholesterol:DMG- | | | | |
| | PEG = 40:18:40.4:1.6 | | | | |
| LQ104-E16b-2 | LQ104 E16b- | 8:1 | 65.98 | 0.073 | 97.2 |
| (DS-f11) | 2:DSPC:cholesterol:DMG- | | | | |
| | PEG = 40:20:38.4:1.6 | | | | |
| LQ104-E16b-2 | LQ104-E16b- | 8:1 | 63.32 | 0.094 | 96.8 |
| (DS-f12) | 2:DSPC:cholesterol:DMG- | | | | |
| | PEG = 40:22:36.4:1.6 | | | | |
| LQ104-E16b-2 | LQ104-E16b- | 8:1 | 63.56 | 0.053 | 97.8 |
| (DS-f13) | 2:DSPC:cholesterol:DMG- | | | | |
| | PEG = 40:24:34.4:1.6 | | | | |
| LQ104-E16b-2 | LQ104-E16b- | 8:1 | 70.45 | 0.105 | 97.4 |
| (DS-f14) | 2:DSPC:cholesterol:DMG- | | | | |
| | PEG = 40:26:32.4:1.6 | | | | |
| LQ104-E16b-2 | LQ104-E16b- | 8:1 | 71.01 | 0.099 | 97.5 |
| (DS-f15) | 2:DSPC:cholesterol:DMG- | | | | |
| | PEG = 40:28:30.4:1.6 | | | | |
| LQ104-E16b-2 | LQ104-E16b- | 8:1 | 90.75 | 0.105 | 97.5 |
| (DS-f16) | 2:DSPC:cholesterol:DMG- | | | | |
| | PEG = 40:30:28.4:1.6 | | | | |
| LQ104-E16b-2 | LQ104-E16b- | 8:1 | 79.58 | 0.113 | 97.2 |
| (DS-f17) | 2:DSPC:cholesterol:DMG- | | | | |
| | PEG = 40:32:26.4:1.6 | | | | |
| LQ104-E16b-2 | LQ104-E16b- | 8:1 | 92.18 | 0.121 | 97.0 |
| (DS-f18) | 2:DSPC:cholesterol:DMG- | | | | |
| | PEG = 40:34:24.4:1.6 | | | | |
| LQ104-E16b-2 | LQ104-E16b- | 8:1 | 84.91 | 0.145 | 97.6 |
| (DS-f19) | 2:DSPC:cholesterol:DMG- | | | | |
| | PEG = 40:36:22.4:1.6 | | | | |
| LQ104-E16b-2 | LQ104-E16b- | 8:1 | 100.2 | 0.157 | 97.0 |
| (DS-f20) | 2:DSPC:cholesterol:DMG- | | | | |
| | PEG = 40:38:20.4:1.6 | | | | |
| LQ104-E16b-2 | LQ104-E16b- | 8:1 | 90.58 | 0.166 | 97.5 |
| (DS-f21) | 2:DSPC:cholesterol:DMG- | | | | |
| | PEG = 40:40:18.4:1.6 | | | | |

It can be seen from Table 7 that the particle size of each group of LNP preparation prepared from LQ104-E16b-2 was between 60 nm and 110 nm; PDI was less than 0.3, specifically between 0.053 and 0.166; and encapsulation efficiency was all above 90% and above 96.8%.

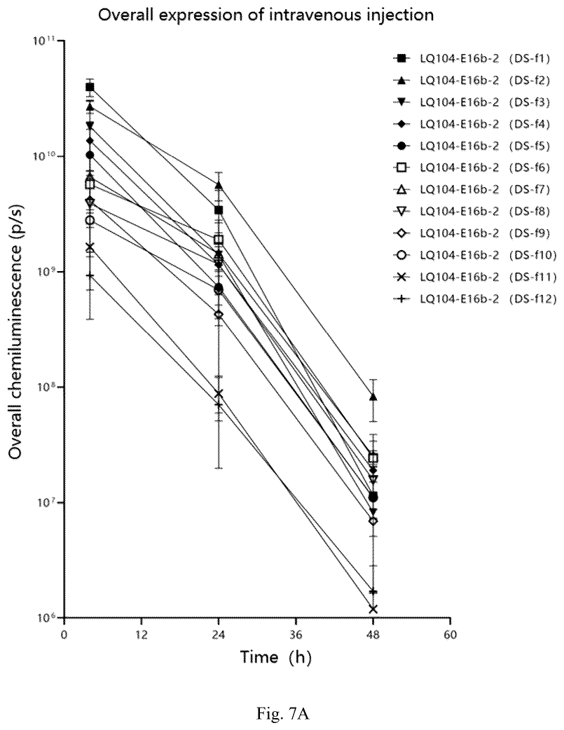

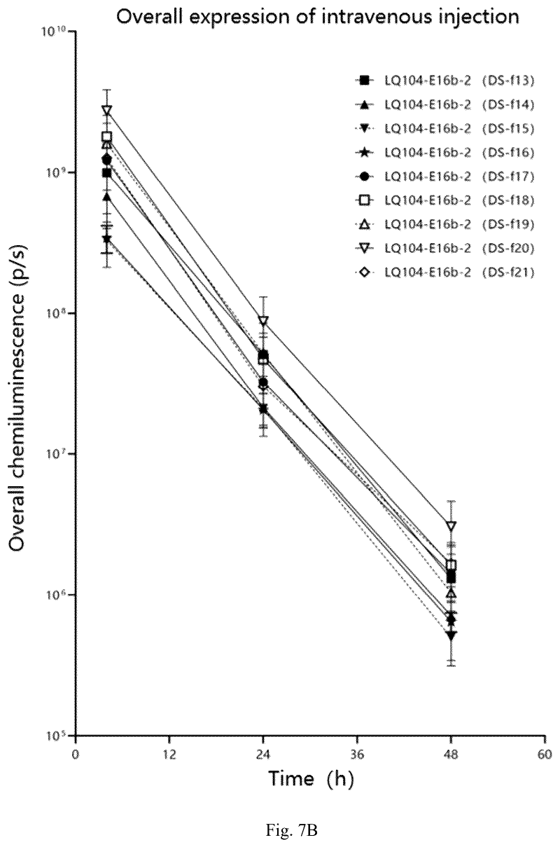

Similarly, referring to the mouse in-vivo experimental method of Example 3, all LNP preparations prepared in this example were injected into female Balb/C mice aged 6 to 8 weeks through the tail vein at a dose of 5 μg/mouse, and the overall luminescence intensity of the liver area was counted by using the same method as Example 3. Test results were shown in FIG. 7A and FIG. 7B as well as Table 8A to Table 8B. It can be seen that the LNP preparation tested in this example had strong expression in mice. Wherein, the overall luminescence intensity of LQ104-E16b-2 (DS-f1) to LQ104-E16b-2 (DS-f10) was relatively high, which indicated that the LNP preparation with the DSPC molar percentage content between 000 and 18% had better in-vivo delivery ability.

TABLE 8A
(AUC data in FIG. 7A)

| Group | AUC (p/s*hour) |
|---|---|
| LQ104-E16b-2 (DS-f1) | 4.74E+11 |
| LQ104-E16b-2 (DS-f2) | 3.97E+11 |
| LQ104-E16b-2 (DS-f3) | 2.13E+11 |
| LQ104-E16b-2 (DS-f4) | 1.63E+11 |
| LQ104-E16b-2 (DS-f5) | 1.19E+11 |
| LQ104-E16b-2 (DS-f6) | 9.89E+10 |
| LQ104-E16b-2 (DS-f7) | 9.79E+10 |
| LQ104-E16b-2 (DS-f8) | 6.45E+10 |
| LQ104-E16b-2 (DS-f9) | 5.09E+10 |
| LQ104-E16b-2 (DS-f10) | 4.34E+10 |
| LQ104-E16b-2 (DS-f11) | 1.83E+10 |
| LQ104-E16b-2 (DS-f12) | 1.09E+10 |

TABLE 8B
(AUC data in FIG. 7B)

| Group | AUC (p/s*hour) |
|---|---|
| LQ104-E16b-2 (DS-f13) | 1.11E+10 |
| LQ104-E16b-2 (DS-f14) | 7.24E+09 |
| LQ104-E16b-2 (DS-f15) | 3.74E+09 |
| LQ104-E16b-2 (DS-f16) | 3.88E+09 |
| LQ104-E16b-2 (DS-f17) | 1.29E+10 |
| LQ104-E16b-2 (DS-f18) | 1.91E+10 |
| LQ104-E16b-2 (DS-f19) | 1.73E+10 |
| LQ104-E16b-2 (DS-f20) | 2.95E+10 |
| LQ104-E16b-2 (DS-f21) | 1.34E+10 |

### Example 7

Unlike Example 6, in this example, phospholipid DSPC in an LNP preparation component was replaced with 1,2-Dioleoyl-sn-glycero-3-phosphoethanolamin (DOPE), and an LNP preparation (encapsulating Fluc mRNA) was prepared according to the same method as Example 1, so as to test a particle size, PDI, and encapsulation efficiency of the LNP preparation prepared by using DOPE as the phospholipid and changing the component from 0% to 22%, as shown in Table 9.

TABLE 9

| | | Nitrogen- | Finished product feature | | |
|---|---|---|---|---|---|
| | Molar percentage content | to- | Particle | | Encapsulation |
| | of preparation components | phosphorus | size | | efficiency |
| Group | (%) | ratio | (nm) | PDI | (%) |
| LQ104-E16b-2 | LQ104-E16b- | 8:1 | 87.90 | 0.130 | 97.8 |
| (DO-f1) | 2:DOPE:cholesterol:DMG- | | | | |
| | PEG = 40:0:58.4:1.6 | | | | |
| LQ104-E16b-2 | LQ104-E16b- | 8:1 | 92.42 | 0.106 | 97.5 |
| (DO-f2) | 2:DOPE:cholesterol:DMG- | | | | |
| | PEG = 40:2:56.4:1.6 | | | | |
| LQ104-E16b-2 | LQ104-E16b- | 8:1 | 79.14 | 0.110 | 97.7 |
| (DO-f3) | 2:DOPE:cholesterol:DMG- | | | | |
| | PEG = 40:4:54.4:1.6 | | | | |
| LQ104-E16b-2 | LQ104-E16b- | 8:1 | 77.79 | 0.123 | 97.4 |
| (DO-f4) | 2:DOPE:cholesterol:DMG- | | | | |
| | PEG = 40:6:52.4:1.6 | | | | |
| LQ104-E16b-2 | LQ104-E16b- | 8:1 | 75.27 | 0.118 | 97.6 |
| (DO-f5) | 2:DOPE:cholesterol:DMG- | | | | |
| | PEG = 40:8:50.4:1.6 | | | | |
| LQ104-E16b-2 | LQ104-E16b- | 8:1 | 79.57 | 0.116 | 97.8 |
| (DO-f6) | 2:DOPE:cholesterol:DMG- | | | | |
| | PEG = 40:10:48.4:1.6 | | | | |
| LQ104-E16b-2 | LQ104-E16b- | 8:1 | 79.55 | 0.126 | 97.9 |
| (DO-f7) | 2:DOPE:cholesterol:DMG- | | | | |
| | PEG = 40:12:46.4:1.6 | | | | |
| LQ104-E16b-2 | LQ104-E16b- | 8:1 | 78.14 | 0.152 | 97.6 |
| (DO-f8) | 2:DOPE:cholesterol:DMG- | | | | |
| | PEG = 40:14:44.4:1.6 | | | | |
| LQ104-E16b-2 | LQ104-E16b- | 8:1 | 80.29 | 0.144 | 97.7 |
| (DO-f9) | 2:DOPE:cholesterol:DMG- | | | | |
| | PEG = 40:16:42.4:1.6 | | | | |
| LQ104-E16b-2 | LQ104-E16b- | 8:1 | 76.70 | 0.154 | 96.8 |
| (DO-f10) | 2:DOPE:cholesterol:DMG- | | | | |
| | PEG = 40:18:40.4:1.6 | | | | |
| LQ104-E16b-2 | LQ104-E16b- | 8:1 | 76.55 | 0.139 | 96.3 |
| (DO-f11) | 2:DOPE:cholesterol:DMG- | | | | |
| | PEG = 40:20:38.4:1.6 | | | | |
| LQ104-E16b-2 | LQ104-E16b- | 8:1 | 75.27 | 0.162 | 96.1 |
| (DO-f12) | 2:DOPE:cholesterol:DMG- | | | | |
| | PEG = 40:22:36.4:1.6 | | | | |

It can be seen from Table 9 that the particle size of the LNP preparation prepared in this example was between 70 nm and 100 nm, with the PDI of less than 0.3 and the encapsulation efficiency of above 96%.

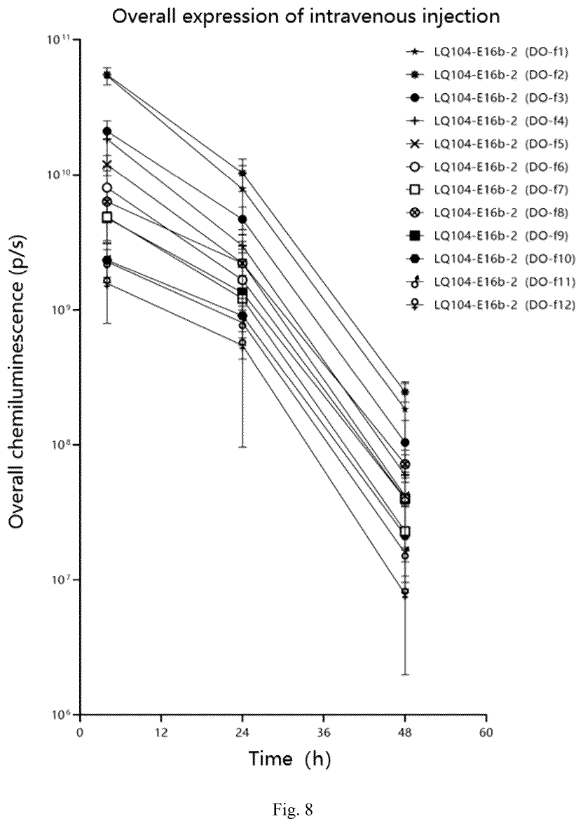

Referring to the mouse in-vivo experimental method of Example 3, each LNP preparation prepared in this example was injected into female Balb/C mice aged 6 to 8 weeks through the tail vein at a dose of 5 g/mouse, and the overall luminescence intensity of the living liver sites of the mice was counted. Test results were shown in FIG. 8 and Table 10. It can be seen that each LNP preparation prepared in this example had strong expression in mice. In addition, we tested various phospholipid molecules and found that the prepared LNP preparations can achieve in-vivo delivery and expression.

TABLE 10
(AUC data in FIG. 8)

| Group | AUC (p/s*hour) |
|---|---|
| LQ104-E16b-2 (DO-f1) | 7.17E+11 |
| LQ104-E16b-2 (DO-f2) | 7.82E+11 |
| LQ104-E16b-2 (DO-f3) | 3.15E+11 |
| LQ104-E16b-2 (DO-f4) | 2.50E+11 |
| LQ104-E16b-2 (DO-f5) | 1.68E+11 |
| LQ104-E16b-2 (DO-f6) | 1.17E+11 |
| LQ104-E16b-2 (DO-f7) | 7.54E+10 |
| LQ104-E16b-2 (DO-f8) | 1.13E+11 |
| LQ104-E16b-2 (DO-f9) | 7.77E+10 |
| LQ104-E16b-2 (DO-f10) | 4.35E+10 |
| LQ104-E16b-2 (DO-f11) | 4.06E+10 |
| LQ104-E16b-2 (DO-f12) | 2.78E+10 |

### Example 8

In this example, LQ104-E16b-2 obtained through Preparation Example 7 was selected as ionizable lipid, an LNP preparation (encapsulating Fluc mRNA) was prepared by referring to the manner in Example 1 according to a molar ratio and a nitrogen-to-phosphorus ratio in Table 11. Different from Example 1, mRNA in this example was diluted in 25 mM of sodium acetate solution with pH of 5.0, and 20 mM of Tris-acetic acid solution with pH of 7.5 was used for dialysis. Particle sizes, PDI, and encapsulation efficiency of all LNP preparations in this example were measured. This example was mainly used to verify an optimal component content of the ionizable lipid in the LNP preparations. We used DSPC with molar percentage contents of 0%, 2%, 4%, and 10% respectively to be fixedly matched with PEG lipid with a molar percentage content of 1.6% to simultaneously screen a content range of ionizable lipids for several specific components (cholesterol was used in this example to make up for the allowance after determining the other three components).

TABLE 11

| | | Nitrogen- | Finished product feature | | |
|---|---|---|---|---|---|
| | Molar percentage content | to- | Particle | | Encapsulation |
| | of preparation components | phosphorus | size | | efficiency |
| Group | (%) | ratio | (nm) | PDI | (%) |
| LQ104-E16b-2 | LQ104-E16b- | 8:1 | 81.61 | 0.206 | 94.9 |
| (ION-f1) | 2:DSPC:cholesterol:DMG- | | | | |
| | PEG = 35:0:63.4:1.6 | | | | |
| LQ104-E16b-2 | LQ104-E16b- | 8:1 | 87.05 | 0.159 | 96.0 |
| (ION-f2) | 2:DSPC:cholesterol:DMG- | | | | |
| | PEG = 40:0:58.4:1.6 | | | | |
| LQ104-E16b-2 | LQ104-E16b- | 8:1 | 101.27 | 0.137 | 95.9 |
| (ION-f3) | 2:DSPC:cholesterol:DMG- | | | | |
| | PEG = 45:0:53.4:1.6 | | | | |
| LQ104-E16b-2 | LQ104-E16b- | 8:1 | 65.44 | 0.197 | 94.8 |
| (ION-f4) | 2:DSPC:cholesterol:DMG- | | | | |
| | PEG = 30:2:66.4:1.6 | | | | |
| LQ104-E16b-2 | LQ104-E16b- | 8:1 | 73.37 | 0.180 | 95.8 |
| (ION-f5) | 2:DSPC:cholesterol:DMG- | | | | |
| | PEG = 35:2:61.4:1.6 | | | | |
| LQ104-E16b-2 | LQ104-E16b- | 8:1 | 85.82 | 0.159 | 96.3 |
| (ION-f6) | 2:DSPC:cholesterol:DMG- | | | | |
| | PEG = 40:2:56.4:1.6 | | | | |
| LQ104-E16b-2 | LQ104-E16b- | 8:1 | 92.43 | 0.142 | 96.4 |
| (ION-f7) | 2:DSPC:cholesterol:DMG- | | | | |
| | PEG-45:2:51.4:1.6 | | | | |
| LQ104-E16b-2 | LQ104-E16b- | 8:1 | 101.67 | 0.109 | 96.4 |
| (ION-f8) | 2:DSPC:cholesterol:DMG- | | | | |
| | PEG = 50:2:46.4:1.6 | | | | |
| LQ104-E16b-2 | LQ104-E16b- | 8:1 | 121.60 | 0.050 | 95.6 |
| (ION-f9) | 2:DSPC:cholesterol:DMG- | | | | |
| | PEG = 60:2:36.4:1.6 | | | | |
| LQ104-E16b-2 | LQ104-E16b- | 8:1 | 72.89 | 0.287 | 95.3 |
| (ION-f10) | 2:DSPC:cholesterol:DMG- | | | | |
| | PEG = 25:4:69.4:1.6 | | | | |
| LQ104-E16b-2 | LQ104-E16b- | 8:1 | 61.04 | 0.174 | 96.0 |
| (ION-f11) | 2:DSPC:cholesterol:DMG- | | | | |
| | PEG = 30:4:64.4:1.6 | | | | |
| LQ104-E16b-2 | LQ104-E16b- | 8:1 | 65.32 | 0.156 | 96.1 |
| (ION-f12) | 2:DSPC:cholesterol:DMG- | | | | |
| | PEG = 35:4:59.4:1.6 | | | | |
| LQ104-E16b-2 | LQ104-E16b- | 8:1 | 70.30 | 0.180 | 96.1 |
| (ION-f13) | 2:DSPC:cholesterol:DMG- | | | | |
| | PEG = 40:4:54.4:1.6 | | | | |
| LQ104-E16b-2 | LQ104-E16b- | 8:1 | 76.55 | 0.154 | 97.3 |
| (ION-f14) | 2:DSPC:cholesterol:DMG- | | | | |
| | PEG = 45:4:49.4:1.6 | | | | |
| LQ104-E16b-2 | LQ104-E16b- | 8:1 | 114.97 | 0.056 | 95.1 |
| (ION-f15) | 2:DSPC:cholesterol:DMG- | | | | |
| | PEG = 60:4:34.4:1.6 | | | | |
| LQ104-E16b-2 | LQ104-E16b- | 8:1 | 59.04 | 0.209 | 97.6 |
| (ION-f16) | 2:DSPC:cholesterol:DMG- | | | | |
| | PEG = 25:10:63.4:1.6 | | | | |
| LQ104-E16b-2 | LQ104-E16b- | 8:1 | 55.40 | 0.155 | 97.2 |
| (ION-f17) | 2:DSPC:cholesterol:DMG- | | | | |
| | PEG = 30:10:58.4:1.6 | | | | |
| LQ104-E16b-2 | LQ104-E16b- | 8:1 | 56.53 | 0.182 | 97.0 |
| (ION-f18) | 2:DSPC:cholesterol:DMG- | | | | |
| | PEG = 35:10:53.4:1.6 | | | | |
| LQ104-E16b-2 | LQ104-E16b- | 8:1 | 63.93 | 0.180 | 97.6 |
| (ION-f19) | 2:DSPC:cholesterol:DMG- | | | | |
| | PEG = 40:10:48.4:1.6 | | | | |
| LQ104-E16b-2 | LQ104-E16b- | 8:1 | 68.27 | 0.151 | 97.0 |
| (ION-f20) | 2:DSPC:cholesterol:DMG- | | | | |
| | PEG = 45:10:43.4:1.6 | | | | |

It can be seen from Table 11 that the particle size of the LNP preparation prepared in this example was between 55 nm and 120 nm, with the PDI of less than 0.3 and the encapsulation efficiency of above 90%.

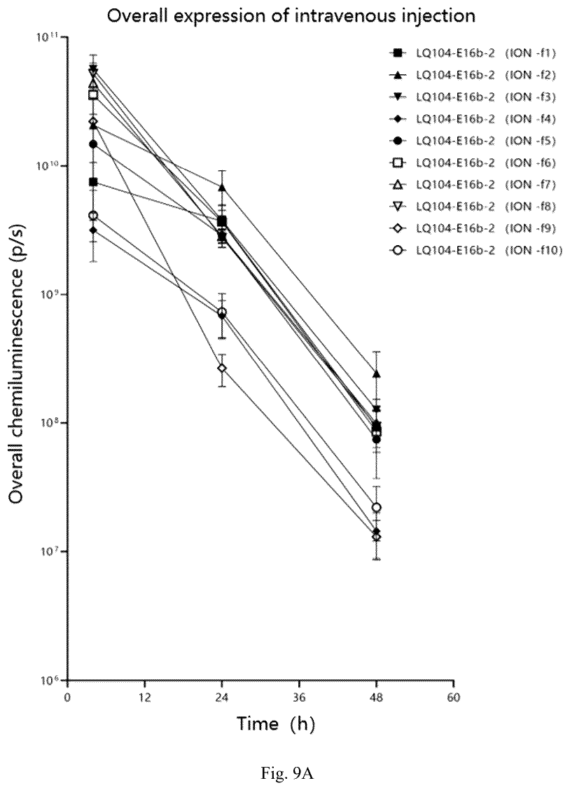

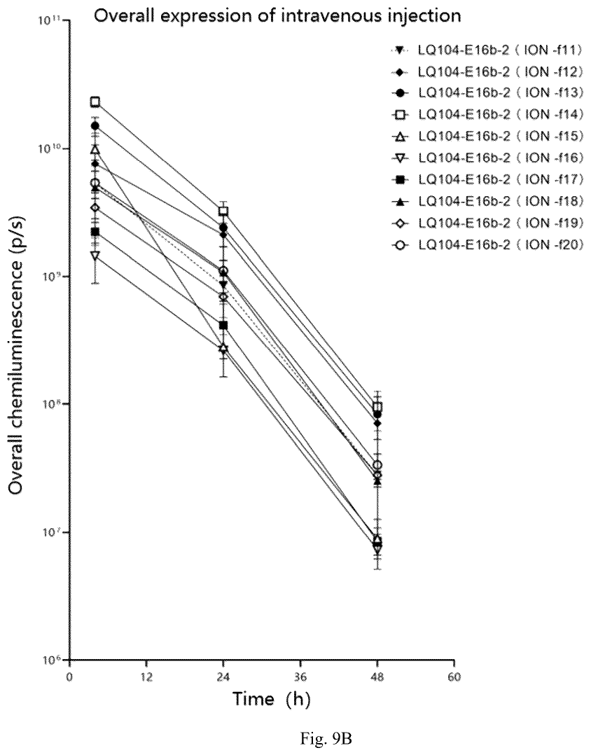

Referring to the mouse in-vivo experimental method of Example 3, all LNP preparations prepared in this example were injected into female Balb/C mice aged 6 to 8 weeks through the tail vein at a dose of 5 g/mouse, and the overall luminescence intensity of the living liver sites of the mice was counted. Test results were shown in FIG. 9A to FIG. 9B and Table 12A to Table 12B. It can be seen that all the LNP preparations prepared in this example had strong expression in mice. Based on our screening tests on the content of ionizable lipid components, results showed that ionizable lipids within the range between 40% and 55% correspond to better in-vivo delivery effect of the LNP preparations.

TABLE 12A
(AUC data in FIG. 9A)

| Group | AUC (p/s*hour) |
|---|---|
| LQ104-E16b-2 (ION -f1) | 1.57E+11 |
| LQ104-E16b-2 (ION -f2) | 3.62E+11 |
| LQ104-E16b-2 (ION -f3) | 6.47E+11 |
| LQ104-E16b-2 (ION -f4) | 4.67E+10 |
| LQ104-E16b-2 (ION -f5) | 2.12E+11 |
| LQ104-E16b-2 (ION -f6) | 4.38E+11 |
| LQ104-E16b-2 (ION -f7) | 5.05E+11 |
| LQ104-E16b-2 (ION -f8) | 5.82E+11 |
| LQ104-E16b-2 (ION -f9) | 2.28E+11 |
| LQ104-E16b-2 (ION -f10) | 5.75E+10 |

TABLE 12B
(AUC data in FIG. 9B)

| Group | AUC (p/s*hour) |
|---|---|
| LQ104-E16b-2 (ION -f11) | 7.23E+10 |
| LQ104-E16b-2 (ION -f12) | 1.23E+11 |
| LQ104-E16b-2 (ION -f13) | 2.05E+11 |
| LQ104-E16b-2 (ION -f14) | 3.05E+11 |
| LQ104-E16b-2 (ION -f15) | 1.05E+11 |
| LQ104-E16b-2 (ION -f16) | 2.03E+10 |
| LQ104-E16b-2 (ION -f17) | 3.15E+10 |
| LQ104-E16b-2 (ION -f18) | 7.36E+10 |
| LQ104-E16b-2 (ION -f19) | 5.00E+10 |
| LQ104-E16b-2 (ION -f20) | 7.84E+10 |

### Example 9

LNP preparations LQ104-E16b-2 (DS-f1) to LQ104-E16b-2 (DS-f7) prepared in Example 6 were stored at 4° C. for 14 days, and their particle sizes and PDI were measured. Results were shown in Table 13.

TABLE 13

| | Sample features | | Increment with | |
|---|---|---|---|---|
| | after placement at | | an immediately | |
| | 4° C. for two weeks | | measured sample | |
| | Particle size | | Particle size | |
| Group | (nm) | PDI | increment (nm) | PDI |
| LQ104-E16b-2 (DS-f1)-- | 86.79 | 0.174 | −3.34 | 0.035 |
| two weeks at 4° C. | | | | |
| LQ104-E16b-2 (DS-f2)-- | 84.43 | 0.187 | 1.91 | 0.106 |
| two weeks at 4° C. | | | | |
| LQ104-E16b-2 (DS-f3)-- | 72.24 | 0.085 | −1.07 | 0.012 |
| two weeks at 4° C. | | | | |
| LQ104-E16b-2 (DS-f4)-- | 71.04 | 0.100 | 0.25 | −0.001 |
| two weeks at 4° C. | | | | |
| LQ104-E16b-2 (DS-f5)-- | 65.25 | 0.076 | −1.37 | −0.025 |
| two weeks at 4° C. | | | | |
| LQ104-E16b-2 (DS-f6)-- | 70.89 | 0.097 | 1.90 | 0.006 |
| two weeks at 4° C. | | | | |
| LQ104-E16b-2 (DS-f7)-- | 66.69 | 0.098 | 0.68 | 0.035 |
| two weeks at 4° C. | | | | |

It can be seen from Table 13 that the LNP preparations prepared by LQl04-E16b-2 according to different DSPC contents showed a particle size variation of less than 5 nm after being stored at 4° C. for 14 days, indicating good sample stability. Similarly, we can also achieve comparable effects by selecting other LNP preparations in the present application.

In addition, the LNP preparations LQ104-E16b-2 (ION-f3), LQ104-E16b-2 (ION-f6), LQ104-E16b-2 (ION-f7), LQ104-E16b-2 (ION-f8), LQ104-E16b-2 (ION-f13), LQ104-E16b-2 (ION-f14), and LQ104-E16b-2 (ION-f20) prepared in Example 8 were divided into 0.3 mL portions and stored at −80° C. It should be noted that each experiment in the present application required the addition of sucrose with a final concentration of 800 as a protective agent during sample freezing, with freezing time of 6 hours or longer to ensure complete freezing of the sample. When the sample was re-thawed, the sample was placed at 4° C. for no less than 90 minutes. By observation, this condition can ensure complete thawing of the LNP preparation. All samples were frozen-thawed for 5 times to measure their particle sizes and PDI. Results were shown in Table 14.

TABLE 14

| | | | Increment with | |
|---|---|---|---|---|
| | Sample features | | an immediately | |
| | after freezing-thawing | | measured sample | |
| | at −80° C. for 5 times | | Particle size | |
| | Particle size | | increment | |
| Group | (nm) | PDI | (nm) | PDI |
| LQ104-E16b-2 | 99.96 | 0.105 | −0.03 | 0.011 |
| (ION-f3)-- | | | | |
| freezing-thawing | | | | |
| for 5 times | | | | |
| LQ104-E16b-2 | 91.79 | 0.349 | 7.61 | 0.209 |
| (ION-f6)-- | | | | |
| freezing-thawing | | | | |
| for 5 times | | | | |
| LQ104-E16b-2 | 90.90 | 0.155 | 0.09 | 0.047 |
| (ION-f7)-- | | | | |
| freezing-thawing | | | | |
| for 5 times | | | | |
| LQ104-E16b-2 | 100.16 | 0.139 | 1.20 | 0.043 |
| (ION-f8)-- | | | | |
| freezing-thawing | | | | |
| for 5 times | | | | |
| LQ104-E16b-2 | 91.22 | 0.371 | 16.02 | 0.257 |
| (ION-f13)-- | | | | |
| freezing-thawing | | | | |
| for 5 times | | | | |
| LQ104-E16b-2 | 81.76 | 0.200 | 0.81 | 0.087 |
| (ION-f14)-- | | | | |
| freezing-thawing | | | | |
| for 5 times | | | | |
| LQ104-E16b-2 | 76.48 | 0.222 | 7.08 | 0.102 |
| (ION-f20)-- | | | | |
| freezing-thawing | | | | |
| for 5 times | | | | |

It can be seen from Table 14 that except for the LQ104-E16b-2 (ION-f13) group, all other groups of LNP preparations showed small particle size variation after freezing-thawing at −80° C. for 5 times, with a variable of less than 10 nm, which preliminarily indicated that the sample stability was relatively good.

### Example 10

An LNP preparation (encapsulating Fluc mRNA) was prepared by referring to the manner in Example 8 according to a molar ratio and a nitrogen-to-phosphorus ratio in Table 15. Similar to Example 8, an mRNA solution in this example was diluted in 25 mM of sodium acetate solution with pH of 5.0, and a solution during dialysis was 20 mM of Tris-acetic acid solution with pH of 7.5. This example was mainly used to verify performance of the LNP preparation of the present application prepared using three components (ionizable lipid, cholesterol, and DMG-PEG) and four components (ionizable lipids, phospholipid, cholesterol, and DMG-PEG), and to investigate a preferred component content of DMG-PEG in the LNP preparation. In this example, we selected a molar percentage content of DMG-PEG within a range of 1.5%-5% for the experiment. A particle size, PDI, and encapsulation efficiency of each LNP were measured, and results were shown in Table 15.

TABLE 15

| | | Nitrogen- | Finished product feature | | |
|---|---|---|---|---|---|
| | Molar percentage content | to- | Particle | | Encapsulation |
| | of preparation components | phosphorus | size | | efficiency |
| Group | (%) | ratio | (nm) | PDI | (%) |
| LQ104-E16b-2 | LQ104-E16b- | 8:1 | 102.27 | 0.130 | 97.5 |
| (PEG-f1) | 2:DSPC:cholesterol:DMG- | | | | |
| | PEG = 40:0:58.4:1.6 | | | | |
| LQ104-E16b-2 | LQ104-E16b- | 8:1 | 83.31 | 0.149 | 97.3 |
| (PEG-f2) | 2:DSPC:cholesterol:DMG- | | | | |
| | PEG = 40:0:58:2 | | | | |
| LQ104-E16b-2 | LQ104-E16b- | 8:1 | 68.84 | 0.168 | 97.6 |
| (PEG-f3) | 2:DSPC:cholesterol:DMG- | | | | |
| | PEG = 40:0:57.5:2.5 | | | | |
| LQ104-E16b-2 | LQ104-E16b- | 8:1 | 65.68 | 0.180 | 97.1 |
| (PEG-f4) | 2:DSPC:cholesterol:DMG- | | | | |
| | PEG = 40:0:57:3 | | | | |
| LQ104-E16b-2 | LQ104-E16b- | 8:1 | 65.99 | 0.220 | 96.9 |
| (PEG-f5) | 2:DSPC:cholesterol:DMG- | | | | |
| | PEG = 40:0:56.5:3.5 | | | | |
| LQ104-E16b-2 | LQ104-E16b- | 8:1 | 63.38 | 0.223 | 97.0 |
| (PEG-f6) | 2:DSPC:cholesterol:DMG- | | | | |
| | PEG = 40:0:56:4 | | | | |
| LQ104-E16b-2 | LQ104-E16b- | 8:1 | 78.59 | 0.230 | 98.1 |
| (PEG-f7) | 2:DSPC:cholesterol:DMG- | | | | |
| | PEG = 40:0:55:5 | | | | |
| LQ104-E16b-2 | LQ104-E16b- | 8:1 | 109.10 | 0.119 | 98.2 |
| (PEG-f8) | 2:DSPC:cholesterol:DMG- | | | | |
| | PEG = 45:0:53.4:1.6 | | | | |
| LQ104-E16b-2 | LQ104-E16b- | 8:1 | 103.80 | 0.113 | 97.6 |
| (PEG-f9) | 2:DSPC:cholesterol:DMG- | | | | |
| | PEG = 45:0:53:2 | | | | |
| LQ104-E16b-2 | LQ104-E16b- | 8:1 | 87.08 | 0.163 | 97.5 |
| (PEG-f10) | 2:DSPC:cholesterol:DMG- | | | | |
| | PEG = 45:0:52.5:2.5 | | | | |
| LQ104-E16b-2 | LQ104-E16b- | 8:1 | 86.75 | 0.190 | 97.5 |
| (PEG-f11) | 2:DSPC:cholesterol:DMG- | | | | |
| | PEG = 45:0:52:3 | | | | |
| LQ104-E16b-2 | LQ104-E16b- | 8:1 | 76.34 | 0.210 | 97.8 |
| (PEG-f12) | 2:DSPC:cholesterol:DMG- | | | | |
| | PEG = 45:0:51.5:3.5 | | | | |
| LQ104-E16b-2 | LQ104-E16b- | 8:1 | 87.00 | 0.234 | 98.6 |
| (PEG-f13) | 2:DSPC:cholesterol:DMG- | | | | |
| | PEG = 45:0:51:4 | | | | |
| LQ104-E16b-2 | LQ104-E16b- | 8:1 | 110.30 | 0.235 | 98.2 |
| (PEG-f14) | 2:DSPC:cholesterol:DMG- | | | | |
| | PEG = 45:0:50:5 | | | | |
| LQ104-E16b-2 | LQ104-E16b- | 8:1 | 95.94 | 0.149 | 98.6 |
| (PEG-f15) | 2:DSPC:cholesterol:DMG- | | | | |
| | PEG = 45:2:51.4:1.6 | | | | |
| LQ104-E16b-2 | LQ104-E16b- | 8:1 | 83.00 | 0.175 | 97.6 |
| (PEG-f16) | 2:DSPC:cholesterol:DMG- | | | | |
| | PEG = 45:2:51:2 | | | | |
| LQ104-E16b-2 | LQ104-E16b- | 8:1 | 92.21 | 0.355 | 97.5 |
| (PEG-f17) | 2:DSPC:cholesterol:DMG- | | | | |
| | PEG = 45:2:50.5:2.5 | | | | |
| LQ104-E16b-2 | LQ104-E16b- | 8:1 | 72.33 | 0.193 | 97.3 |
| (PEG-f18) | 2:DSPC:cholesterol:DMG- | | | | |
| | PEG = 45:2:50:3 | | | | |
| LQ104-E16b-2 | LQ104-E16b- | 8:1 | 68.00 | 0.231 | 97.4 |
| (PEG-f19) | 2:DSPC:cholesterol:DMG- | | | | |
| | PEG = 45:2:49.5:3.5 | | | | |
| LQ104-E16b-2 | LQ104-E16b- | 8:1 | 68.97 | 0.235 | 97.9 |
| (PEG-f20) | 2:DSPC:cholesterol:DMG- | | | | |
| | PEG = 45:2:49:4 | | | | |
| LQ104-E16b-2 | LQ104-E16b- | 8:1 | 75.74 | 0.292 | 97.6 |
| (PEG-f21) | 2:DSPC:cholesterol:DMG- | | | | |
| | PEG = 45:2:48:5 | | | | |

It can be seen from Table 15 that regardless of whether the LQ104 series was prepared from three or four components, its particle size was between 60 nm and 115 nm, PDI was less than 0.3, and encapsulation efficiency was above 9600.

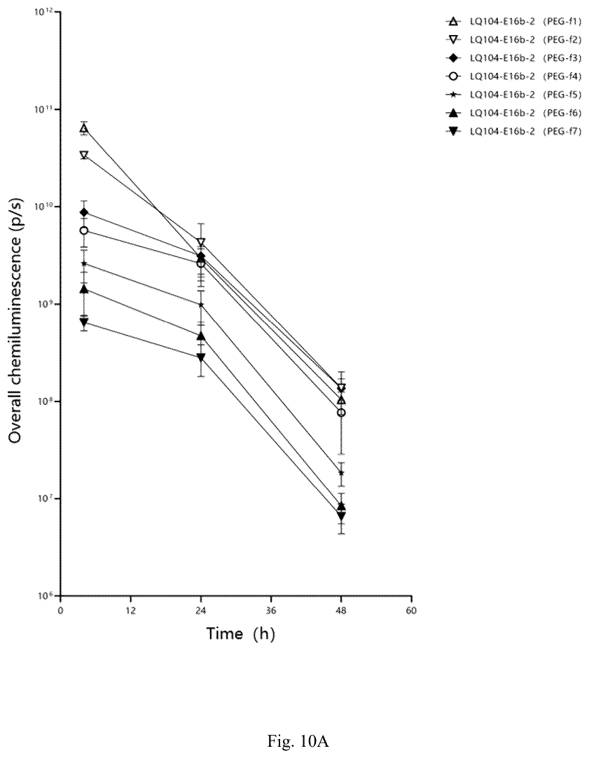

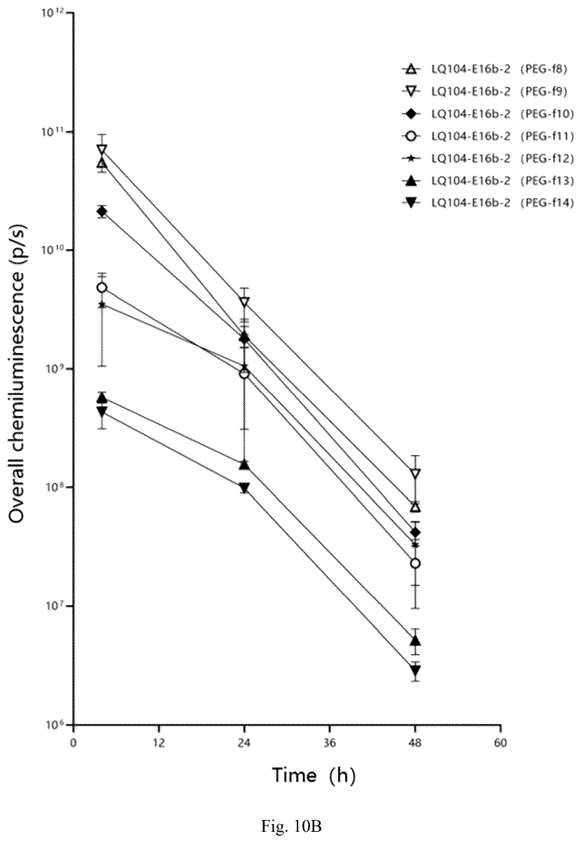

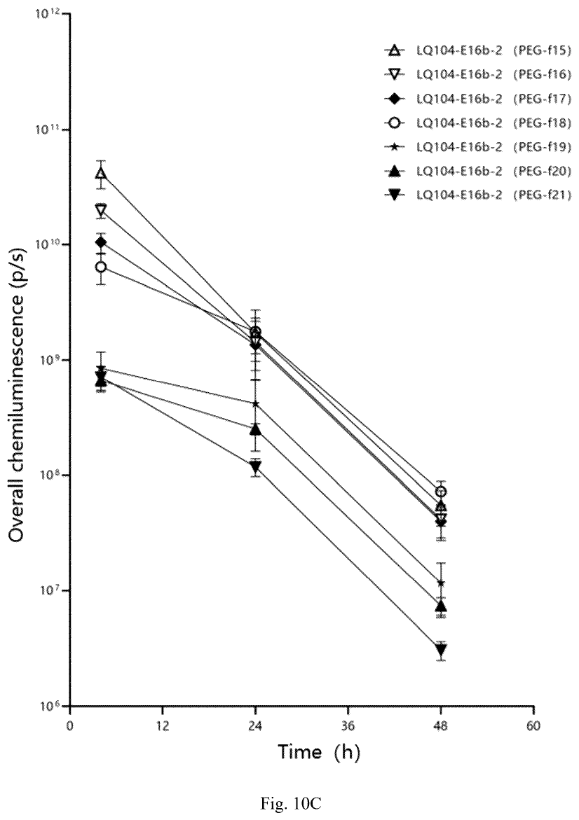

Referring to the mouse in-vivo experimental method of Example 3, all groups of LNP preparations in this example were injected into female Balb/C mice aged 6 to 8 weeks through the tail vein at a dose of 5 μg/mouse, and the overall luminescence intensity of the living liver sites of the mice was counted. Test results were shown in FIG. 10A, FIG. 10B and FIG. 10C as well as Table 16A to Table 16C. It can be seen that all the LNP preparations in this example had strong expression in mice. Moreover, it was also indicated by the experiments that have been carried out that the expression effect was better when the molar percentage content of the PEG lipid was between 1.5% and 2.5%; more preferably, the molar percentage content of the PEG lipid was in a range from 1.5% to 2.0%. In addition, we tested various PEG lipid and found that the prepared LNP preparations can achieve in-vivo delivery and expression.

TABLE 16A
(AUC data in FIG. 10A)

| Group | AUC (p/s*hour) |
|---|---|
| LQ104-E16b-2 (PEG-f1) | 7.15E+11 |
| LQ104-E16b-2 (PEG-f2) | 4.36E+11 |
| LQ104-E16b-2 (PEG-f3) | 1.58E+11 |
| LQ104-E16b-2 (PEG-f4) | 1.15E+11 |
| LQ104-E16b-2 (PEG-f5) | 4.81E+10 |
| LQ104-E16b-2 (PEG-f6) | 2.49E+10 |
| LQ104-E16b-2 (PEG-f7) | 1.27E+10 |

TABLE 16B
(AUC data in FIG. 10B)

| Group | AUC (p/s*hour) |
|---|---|
| LQ104-E16b-2 (PEG-f8) | 5.97E+11 |
| LQ104-E16b-2 (PEG-f9) | 7.88E+11 |
| LQ104-E16b-2 (PEG-f10) | 2.53E+11 |
| LQ104-E16b-2 (PEG-f11) | 6.88E+10 |
| LQ104-E16b-2 (PEG-f12) | 5.90E+10 |
| LQ104-E16b-2 (PEG-f13) | 9.23E+09 |
| LQ104-E16b-2 (PEG-f14) | 6.51E+09 |

TABLE 16C
(AUC data in FIG. 10C)

| Group | AUC (p/s*hour) |
|---|---|
| LQ104-E16b-2 (PEG-f15) | 4.62E+11 |
| LQ104-E16b-2 (PEG-f16) | 2.30E+11 |
| LQ104-E16b-2 (PEG-f17) | 1.35E+11 |
| LQ104-E16b-2 (PEG-f18) | 1.04E+11 |
| LQ104-E16b-2 (PEG-f19) | 1.78E+10 |
| LQ104-E16b-2 (PEG-f20) | 1.23E+10 |
| LQ104-E16b-2 (PEG-f21) | 9.78E+09 |

### Example 11

An LNP preparation (encapsulating Fluc mRNA) was prepared by referring to the manner in Example 8 according to a molar ratio and a nitrogen-to-phosphorus ratio in Table 17. This example was mainly used to verify performance of LNP preparations prepared with different nitrogen-to-phosphorus ratios. We prepared the LNP preparations by setting the nitrogen-to-phosphorus ratio to be 4:1 to 8:1 respectively based on an optimal range of molar percentage contents of ionizable lipid and DSPC verified in the aforementioned examples. A particle size, PDI, and encapsulation efficiency of each LNP preparation were measured, and results were shown in Table 17.

TABLE 17

| | | Nitrogen- | Finished product feature | | |
|---|---|---|---|---|---|
| | Molar percentage content | to- | Particle | | Encapsulation |
| | of preparation | phosphorus | size | | efficiency |
| Group | components (%) | ratio | (nm) | PDI | (%) |
| LQ104E16b-2 | LQ104-E16b- | 4:1 | 61.63 | 0.178 | 97.4 |
| (NP-f1) | 2:DSPC:cholesterol:DMG- | | | | |
| | PEG = 40:0:58:2 | | | | |
| LQ104E16b-2 | LQ104-E16b- | 5:1 | 102.67 | 0.162 | 97.3 |
| (NP-f2) | 2:DSPC:cholesterol:DMG- | | | | |
| | PEG = 40:0:58:2 | | | | |
| LQ104- | LQ104-E16b- | 6:1 | 95.22 | 0.153 | 97.1 |
| E16b-2 | 2:DSPC:cholesterol:DMG- | | | | |
| (NP-f3) | PEG = 40:0:58:2 | | | | |
| LQ104- | LQ104-E16b- | 8:1 | 86.73 | 0.171 | 96.7 |
| E16b-2 | 2:DSPC:cholesterol:DMG- | | | | |
| (NP-f4) | PEG = 40:0:58:2 | | | | |
| LQ104- | LQ104-E16b- | 4:1 | 107.67 | 0.115 | 96.9 |
| E16b-2 | 2:DSPC:cholesterol:DMG- | | | | |
| (NP-f5) | PEG = 45:0:53:2 | | | | |
| LQ104- | LQ104-E16b- | 5:1 | 105.97 | 0.129 | 96.8 |
| E16b-2 | 2:DSPC:cholesterol:DMG- | | | | |
| (NP-f6) | PEG = 45:0:53:2 | | | | |
| LQ104- | LQ104-E16b- | 6:1 | 101.70 | 0.130 | 96.4 |
| E16b-2 | 2:DSPC:cholesterol:DMG- | | | | |
| (NP-f7) | PEG = 45:0:53:2 | | | | |
| LQ104- | LQ104-E16b- | 8:1 | 119.17 | 0.139 | 97.1 |
| E16b-2 | 2:DSPC:cholesterol:DMG- | | | | |
| (NP-f8) | PEG = 45:0:53:2 | | | | |
| LQ104- | LQ104-E16b- | 4:1 | 119.87 | 0.132 | 97.7 |
| E16b-2 | 2:DSPC:cholesterol:DMG- | | | | |
| (NP-f9) | PEG = 45:2:51.4:1.6 | | | | |
| LQ104- | LQ104-E16b- | 5:1 | 99.24 | 0.130 | 97.4 |
| E16b-2 | 2:DSPC:cholesterol:DMG- | | | | |
| (NP-f10) | PEG = 45:2:51.4:1.6 | | | | |
| LQ104- | LQ104-E16b- | 6:1 | 95.28 | 0.142 | 98.7 |
| E16b-2 | 2:DSPC:cholesterol:DMG- | | | | |
| (NP-f11) | PEG = 45:2:51.4:1.6 | | | | |
| LQ104- | LQ104-E16b- | 8:1 | 93.22 | 0.132 | 98.7 |
| E16b-2 | 2:DSPC:cholesterol:DMG- | | | | |
| (NP-f12) | PEG = 45:2:51.4:1.6 | | | | |

It can be seen from Table 17 that the particle size of the LNP preparation in this example was between 60 nm and 120 nm, with the PDI of less than 0.3 and the encapsulation efficiency of above 9600.

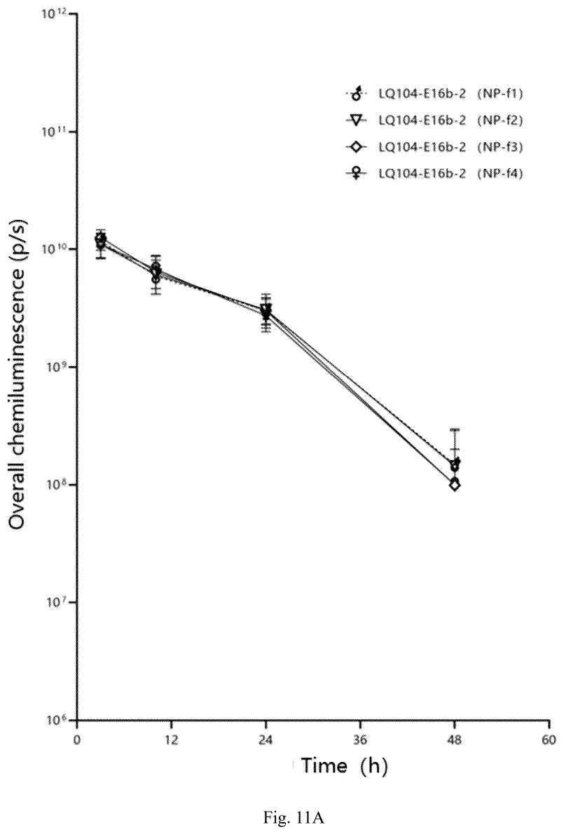

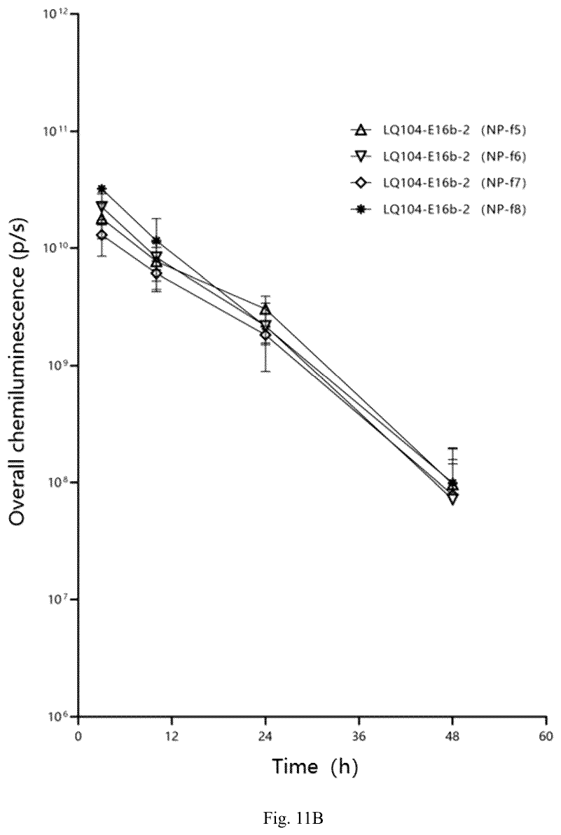

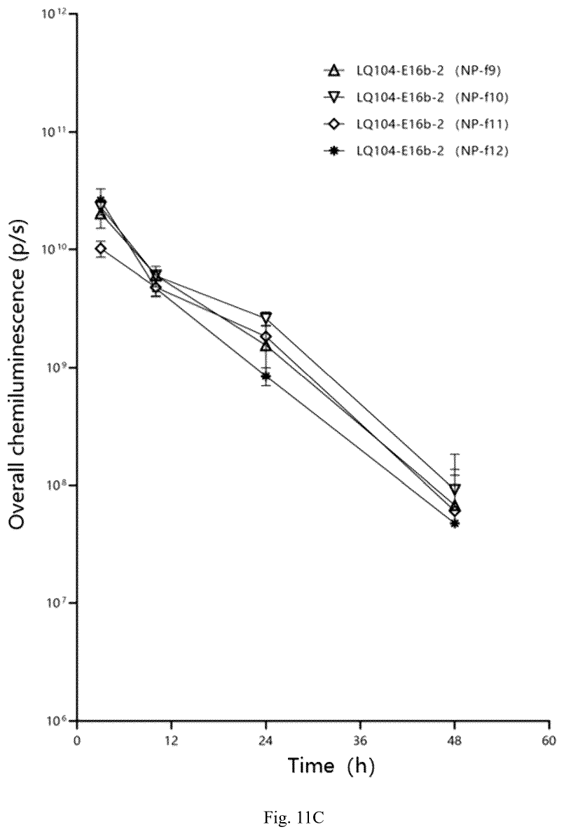

Referring to the mouse in-vivo experimental method of Example 3, all groups of LNP preparations in this example were injected into female Balb/C mice aged 6 to 8 weeks through the tail vein at a dose of 5 μg/mouse, and the overall luminescence intensity of the living liver sites of the mice was counted. Test results were shown in FIG. 11A, FIG. 11B and FIG. 11C as well as Table 18A to Table 18C. It can be seen that the LNP preparation in this example had strong expression in mice when the nitrogen-to-phosphorus ratio was in a range of 4:1 to 8:1.

TABLE 18A
(AUC data in FIG. 11A)

| Group | AUC (p/s*hour) |
|---|---|
| LQ104-E16b-2(NP-f1) | 1.63E+11 |
| LQ104-E16b-2(NP-f2) | 1.63E+11 |
| LQ104-E16b-2(NP-f3) | 1.70E+11 |
| LQ104-E16b-2(NP-f4) | 1.63E+11 |

TABLE 18B
(AUC data in FIG. 11B)

| Group | AUC (p/s*hour) |
|---|---|
| LQ104-E16b-2(NP-f5) | 2.03E+11 |
| LQ104-E16b-2(NP-f6) | 2.09E+11 |
| LQ104-E16b-2(NP-f7) | 1.45E+11 |
| LQ104-E16b-2(NP-f8) | 2.77E+11 |

TABLE 18C
(AUC data in FIG. 11C)

| Group | AUC (p/s*hour) |
|---|---|
| LQ104-E16b-2(NP-f9) | 1.65E+11 |
| LQ104-E16b-2(NP-f10) | 1.95E+11 |
| LQ104-E16b-2(NP-f11) | 1.22E+11 |
| LQ104-E16b-2(NP-f12) | 1.58E+11 |

### Example 12

An LNP preparation (encapsulating Fluc mRNA) was prepared by referring to the manner in Example 8 according to a molar ratio and a nitrogen-to-phosphorus ratio in Table 19. In this example, ionizable lipids prepared in Preparation Example 3 to Preparation Example 17 were selected, and this example was mainly used to verify performance of the LNP preparation with three components (ionizable lipid, cholesterol, and DMG-PEG) in the present application. A particle size, PDI, and encapsulation efficiency of each LNP were measured, and results were shown in Table 19.

TABLE 19

| | | Nitrogen- | Finished product feature | | |
|---|---|---|---|---|---|
| | Molar percentage content | to- | Particle | | Encapsulation |
| | of preparation | phosphorus | size | | efficiency |
| Group | components (%) | ratio | (nm) | PDI | (%) |
| LQ104(f1) | LQ104:DSPC:cholesterol:DMG- | 8:1 | 64.13 | 0.129 | 97.5 |
| | PEG = 40:11:47.4:1.6 | | | | |
| LQ104(f2) | LQ104:DSPC:cholesterol:DMG- | 8:1 | 88.20 | 0.113 | 96.7 |
| | PEG = 45:0:53:2 | | | | |
| LQ104- | LQ104-E15b- | 8:1 | 86.30 | 0.158 | 96.3 |
| E15b-1(f2) | 1:DSPC:cholesterol:DMG- | | | | |
| | PEG = 45:0:53:2 | | | | |
| LQ104- | LQ104-E15b- | 8:1 | 86.96 | 0.167 | 97.6 |
| E15b-2(f2) | 2:DSPC:cholesterol:DMG- | | | | |
| | PEG = 45:0:53:2 | | | | |
| LQ104- | LQ104-E15b- | 8:1 | 70.17 | 0.167 | 96.7 |
| E15b-3(f2) | 3:DSPC:cholesterol:DMG- | | | | |
| | PEG = 45:0:53:2 | | | | |
| LQ104- | LQ104-E16b- | 8:1 | 83.35 | 0.141 | 96.7 |
| E16b-1(f2) | 1:DSPC:cholesterol:DMG- | | | | |
| | PEG = 45:0:53:2 | | | | |
| LQ104- | LQ104-E16b- | 8:1 | 88.77 | 0.124 | 97.2 |
| E16b-2(f2) | 2:DSPC:cholesterol:DMG- | | | | |
| | PEG = 45:0:53:2 | | | | |
| LQ104- | LQ104-E16b- | 8:1 | 89.89 | 0.124 | 97.2 |
| E16b-2(f3) | 2:DSPC:cholesterol:DMG- | | | | |
| | PEG = 50:0:48:2 | | | | |
| LQ104- | LQ104-E16b- | 8:1 | 110.13 | 0.113 | 96.2 |
| E16b-2(f4) | 2:DSPC:cholesterol:DMG- | | | | |
| | PEG = 55:0:43:2 | | | | |
| LQ104- | LQ104-E16b- | 8:1 | 127.90 | 0.086 | 95.7 |
| E16b-2(f5) | 2:DSPC:cholesterol:DMG- | | | | |
| | PEG = 60:0:38:2 | | | | |
| LQ104- | LQ104-E16b- | 8:1 | 76.09 | 0.150 | 95.4 |
| E16b-3(f2) | 3:DSPC:cholesterol:DMG- | | | | |
| | PEG = 45:0:53:2 | | | | |
| LQ104- | LQ104-E17b- | 8:1 | 81.62 | 0.146 | 95.0 |
| E17b-1(f2) | 1:DSPC:cholesterol:DMG- | | | | |
| | PEG = 45:0:53:2 | | | | |
| LQ104- | LQ104-E17b- | 8:1 | 79.39 | 0.133 | 96.2 |
| E17b-2(f2) | 2:DSPC:cholesterol:DMG- | | | | |
| | PEG = 45:0:53:2 | | | | |
| LQ104- | LQ104-E17b- | 8:1 | 82.69 | 0.161 | 95.6 |
| E17b-3(f2) | 3:DSPC:cholesterol:DMG- | | | | |
| | PEG = 45:0:53:2 | | | | |
| LQ104- | LQ104-E17b- | 8:1 | 86.69 | 0.132 | 96.2 |
| E17b-4(f2) | 4:DSPC:cholesterol:DMG- | | | | |
| | PEG = 45:0:53:2 | | | | |
| LQ104- | LQ104-E18b- | 8:1 | 83.64 | 0.135 | 94.9 |
| E18b-2(f2) | 2:DSPC:cholesterol:DMG- | | | | |
| | PEG = 45:0:53:2 | | | | |
| LQ104- | LQ104-E18b- | 8:1 | 87.32 | 0.155 | 96.0 |
| E18b-3(f2) | 3:DSPC:cholesterol:DMG- | | | | |
| | PEG = 45:0:53:2 | | | | |
| LQ104- | LQ104- | 8:1 | 69.70 | 0.135 | 95.6 |
| H3(f2) | H3:DSPC:cholesterol:DMG- | | | | |
| | PEG = 45:0:53:2 | | | | |

It can be seen from Table 19 that the particle sizes of the LNP preparations in this example were all between 60 nm and 130 nm, with the PDI of less than 0.3 and the encapsulation efficiency of above 90%.

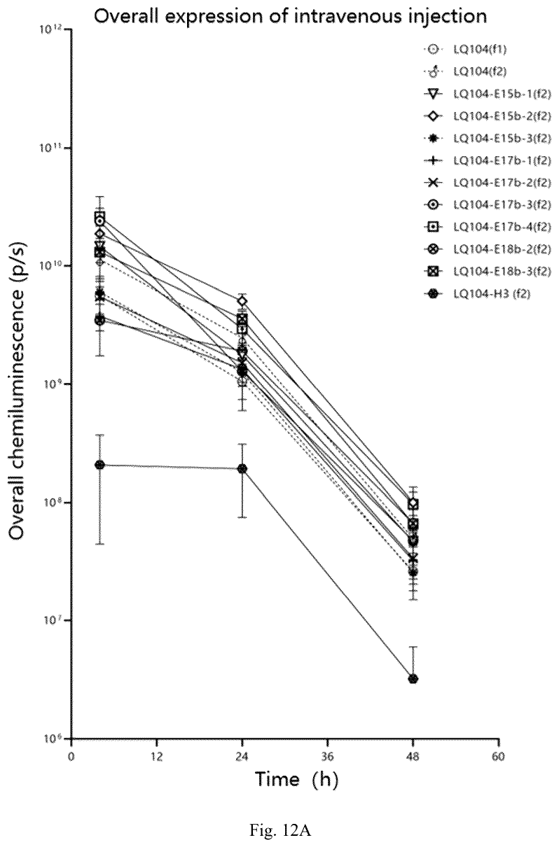

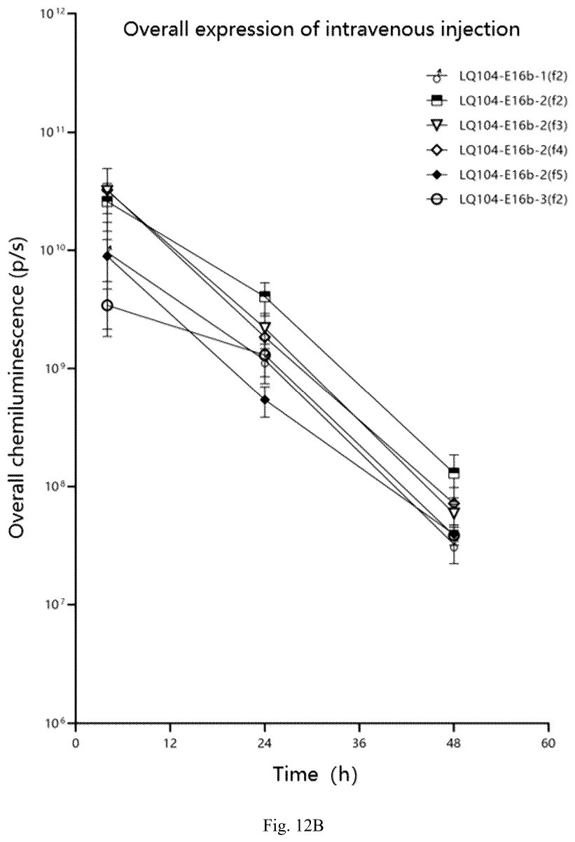

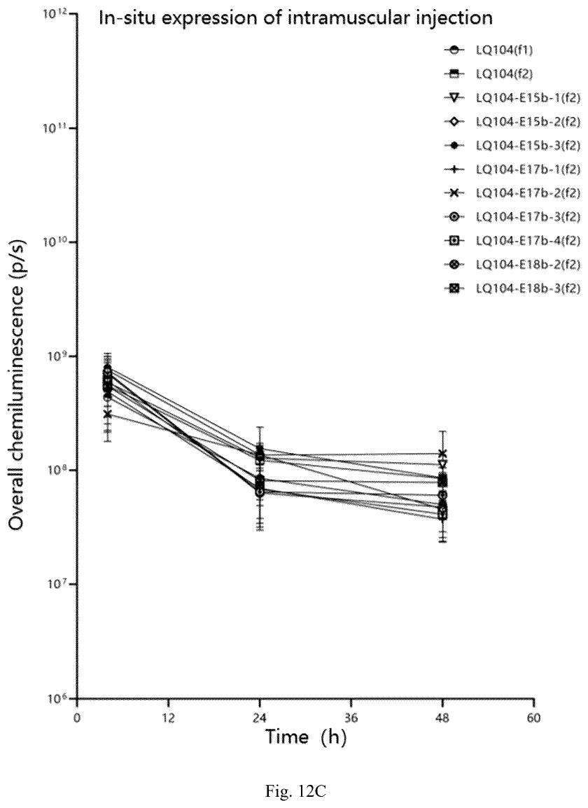

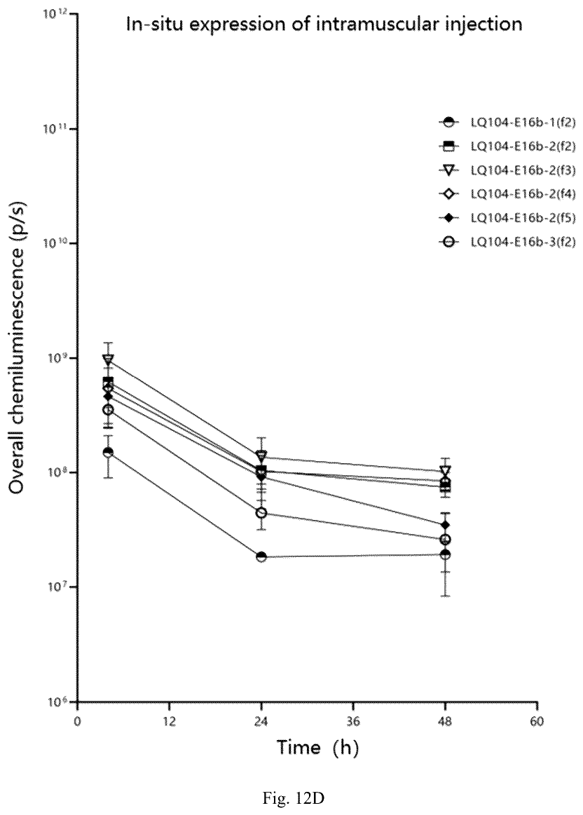

Referring to the mouse in-vivo experimental method of Example 3, each group of LNP preparations in this example was injected into female Balb/C mice aged 6 to 8 weeks through tail vein injection or intramuscular injection of the lower limbs at a dose of 5 μg/mouse, and overall luminescence intensity of the liver (a main expression area when administered through tail vein injection) or lower limb administration site (an expression area mainly focused when administered through intramuscular injection of the lower limbs) was counted. Test results were shown in FIG. 12A to FIG. 12D and Table 20A to Table 20D. Wherein, FIG. 12A and FIG. 12B showed the luminescence statistical results of the liver sites in mice after intravenous injection administration, while FIG. 12C and FIG. 12D showed the luminescence statistical results of the lower limb administration sites in mice after intramuscular injection administration. It can be seen from FIG. 12A to FIG. 12D that all the LNP preparations in this example had strong expression in mice.

TABLE 20A
(AUC data in FIG. 12A)

| Group | AUC (p/s*hour) |
|---|---|
| LQ104(f1) | 7.98E+10 |
| LQ104(f2 | 1.68E+11 |
| LQ104-E15b-1(f2) | 1.85E+11 |
| LQ104-E15b-2(f2) | 3.00E+11 |
| LQ104-E15b-3(f2) | 8.85E+10 |
| LQ104-E17b-1(f2) | 6.78E+10 |
| LQ104-E17b-2(f2) | 8.89E+10 |
| LQ104-E17b-3(f2) | 2.68E+11 |
| LQ104-E17b-4(f2) | 3.26E+11 |
| LQ104-E18b-2(f2) | 7.76E+10 |
| LQ104-E18b-3(f2) | 2.11E+11 |

TABLE 20B
(AUC data in FIG. 12B)

| Group | AUC (p/s*hour) |
|---|---|
| LQ104-E16b-1(f2) | 1.22E+11 |
| LQ104-E16b-2(f2) | 3.50E+11 |
| LQ104-E16b-2(f3) | 3.69E+11 |
| LQ104-E16b-2(f4) | 3.67E+11 |
| LQ104-E16b-2(f5) | 1.01E+11 |
| LQ104-E16b-3(f2) | 6.34E+10 |

TABLE 20C
(AUC data in FIG. 12C)

| Group | AUC (p/s*hour) |
|---|---|
| LQ104(f1) | 6.83E+09 |
| LQ104(f2) | 9.21E+09 |
| LQ104-E15b-1(f2) | 1.01E+10 |
| LQ104-E15b-2(f2) | 1.11E+10 |
| LQ104-E15b-3(f2) | 1.24E+10 |
| LQ104-E17b-1(f2) | 6.86E+09 |
| LQ104-E17b-2(f2) | 7.77E+09 |
| LQ104-E17b-3(f2) | 9.34E+09 |
| LQ104-E17b-4(f2) | 8.06E+09 |
| LQ104-E18b-2(f2) | 8.96E+09 |
| LQ104-E18b-3(f2) | 8.19E+09 |

TABLE 20D
(AUC data in FIG. 12D)

| Group | AUC (p/s*hour) |
|---|---|
| LQ104-E16b-1(f2) | 2.13E+09 |
| LQ104-E16b-2(f2) | 9.36E+09 |
| LQ104-E16b-2(f3) | 1.38E+10 |
| LQ104-E16b-2(f4) | 8.67E+09 |
| LQ104-E16b-2(f5) | 7.04E+09 |
| LQ104-E16b-3(f2) | 4.82E+09 |

### Example 13

In this example, LQ104-E16b-2 obtained through Preparation Example 7 was selected as the ionizable lipid, 16 LNP preparations (encapsulating Fluc mRNA) were prepared by referring to the manner in Example 8 according to a molar ratio and a nitrogen-to-phosphorus ratio in Table 21, and their particle sizes, PDI, and encapsulation efficiency were measured.

TABLE 21

| | | Nitrogen- | Finished product feature | | |
|---|---|---|---|---|---|
| | Molar percentage content | to- | Particle | | Encapsulation |
| | of preparation | phosphorus | size | | efficiency |
| Group | components (%) | ratio | (nm) | PDI | (%) |
| LQ104- | LQ104-E16b- | 8:1 | 293.40 | 0.383 | 97.2 |
| E16b-2 | 2:DSPC:cholesterol:DMG- | | | | |
| (PEG-f1) | PEG = 45:0:54.75:0.25 | | | | |
| LQ104- | LQ104-E16b- | 8:1 | 144.23 | 0.196 | 98.0 |
| E16b-2 | 2:DSPC:cholesterol:DMG- | | | | |
| (PEG-f2) | PEG = 45:0:54.5:0.5 | | | | |
| LQ104- | LQ104-E16b- | 8:1 | 127.00 | 0.123 | 98.3 |
| E16b-2 | 2:DSPC:cholesterol:DMG- | | | | |
| (PEG-f3) | PEG = 45:0:54.25:0.75 | | | | |
| LQ104- | LQ104-E16b- | 8:1 | 101.30 | 0.088 | 98.0 |
| E16b-2 | 2:DSPC:cholesterol:DMG- | | | | |
| (PEG-f4) | PEG = 45:0:54:1 | | | | |
| LQ104- | LQ104-E16b- | 8:1 | 82.46 | 0.141 | 96.7 |
| E16b-2 | 2:DSPC:cholesterol:DMG- | | | | |
| (PEG-f5) | PEG = 45:0:53.4:1.6 | | | | |
| LQ104- | LQ104-E16b- | 8:1 | 87.72 | 0.181 | 96.5 |
| E16b-2 | 2:DSPC:cholesterol:DMG- | | | | |
| (PEG-f6) | PEG = 45:0:53:2 | | | | |
| LQ104- | LQ104-E16b- | 8:1 | 75.55 | 0.269 | 95.6 |
| E16b-2 | 2:DSPC:cholesterol:DMG- | | | | |
| (PEG-f7) | PEG = 45:0:52.5:2.5 | | | | |
| LQ104- | LQ104-E16b- | 8:1 | 84.72 | 0.246 | 95.7 |
| E16b-2 | 2:DSPC:cholesterol:DMG- | | | | |
| (PEG-f8) | PEG = 45:0:52:3 | | | | |
| LQ104- | LQ104-E16b- | 8:1 | 160.60 | 0.227 | 99.1 |
| E16b-2 | 2:DSPC:cholesterol:DMG- | | | | |
| (PEG-f9) | PEG = 45:2:52.75:0.25 | | | | |
| LQ104- | LQ104-E16b- | 8:1 | 128.10 | 0.183 | 98.3 |
| E16b-2 | 2:DSPC:cholesterol:DMG- | | | | |
| (PEG-f10) | PEG = 45:2:52.5:0.5 | | | | |
| LQ104- | LQ104 E16b- | 8:1 | 104.20 | 0.108 | 98.5 |
| E16b-2 | 2:DSPC:cholesterol:DMG- | | | | |
| (PEG-f11) | PEG = 45:2:52.25:0.75 | | | | |
| LQ104- | LQ104-E16b- | 8:1 | 92.91 | 0.135 | 98.0 |
| E16b-2 | 2:DSPC:cholesterol:DMG- | | | | |
| (PEG-f12) | PEG = 45:2:52:1 | | | | |
| LQ104- | LQ104-E16b- | 8:1 | 82.06 | 0.173 | 96.9 |
| E16b-2 | 2:DSPC:cholesterol:DMG- | | | | |
| (PEG-f13) | PEG = 45:2:51.4:1.6 | | | | |
| LQ104- | LQ104-E16b- | 8:1 | 75.43 | 0.165 | 98.6 |
| E16b-2 | 2:DSPC:cholesterol:DMG- | | | | |
| (PEG-f14) | PEG = 45:2:51:2 | | | | |
| LQ104- | LQ104-E16b- | 8:1 | 77.54 | 0.227 | 96.8 |
| E16b-2 | 2:DSPC:cholesterol:DMG- | | | | |
| (PEG-f15) | PEG = 45:2:50.5:2.5 | | | | |
| LQ104- | LQ104-E16b- | 8:1 | 71.03 | 0.222 | 95.7 |
| E16b-2 | 2:DSPC:cholesterol:DMG- | | | | |
| (PEG-f16) | PEG = 45:2:50:3 | | | | |

It can be seen from Table 21 that the particle size of each group of LNP preparations prepared from LQ104-E16b-2 was between 70 nm and 150 nm when DMG-PEG2000 was between 0.5 mol % and 3 mol %; PDI was less than 0.3; and encapsulation efficiency was all above 90% and above 95.6%.

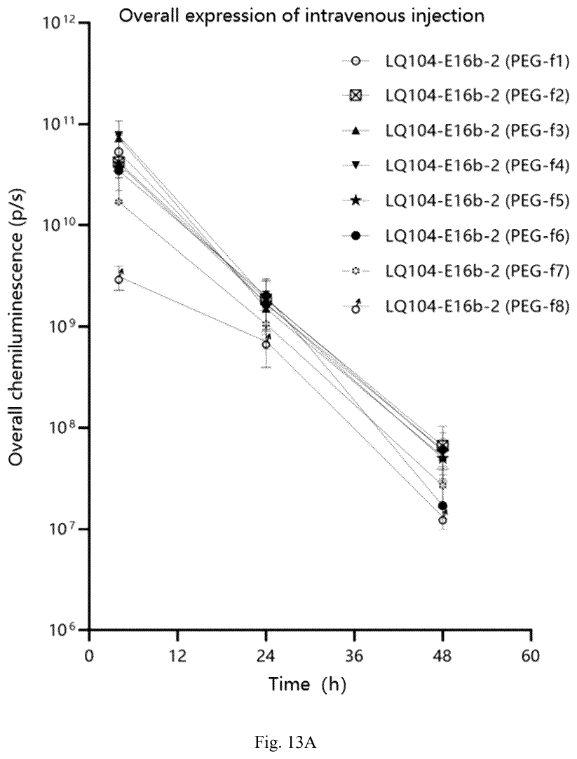

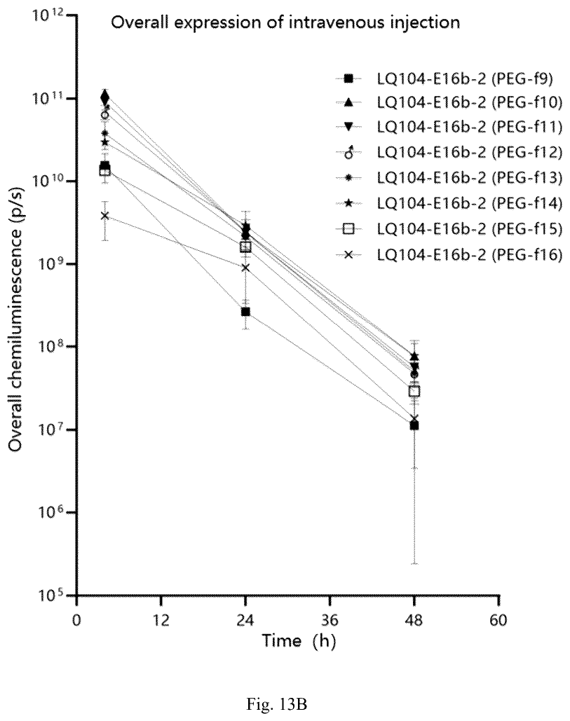

Similarly, referring to the mouse in-vivo experimental method of Example 3, all LNP preparations prepared in this example were injected into female Balb/C mice aged 6 to 8 weeks through the tail vein at a dose of 5 ag/mouse, and the overall luminescence intensity of the liver area was counted by using the same method as Example 3. Test results were shown in Table 22 and Table 23 as well as FIG. 13A and FIG. 13B. It can be seen that the LNP preparation tested in this example had strong expression in mice. Wherein, the overall luminescence intensity of LQ104-E16b-2 (PEG-f1) to LQ104-E16b-2 (PEG-f6) was higher in the 0% DSPC group, indicating that the LNP preparation corresponding to the PEG lipid with the molar percentage content ranging from 0.25% to 2% in this group had better in-vivo delivery ability; and overall luminescence intensity of LQ104-E16b-2 (PEG-f9) to LQ104-E16b-2 (PEG-f15) was higher in the 2% DSPC group, indicating that the LNP preparation corresponding to the PEG lipid with the molar percentage content ranging from 0.25% to 2.5% in this group had better in-vivo delivery ability.

TABLE 22
(AUC data in FIG. 13A)

| Group | AUC (p/s*hour) |
|---|---|
| LQ104-E16b-2 (PEG-f1) | 5.71E+11 |
| LQ104-E16b-2 (PEG-f2) | 4.63E+11 |
| LQ104-E16b-2 (PEG-f3) | 7.64E+11 |
| LQ104-E16b-2 (PEG-f4) | 8.18E+11 |
| LQ104-E16b-2 (PEG-f5) | 4.43E+11 |
| LQ104-E16b-2 (PEG-f6) | 3.92E+11 |
| LQ104-E16b-2 (PEG-f7) | 1.94E+11 |
| LQ104-E16b-2 (PEG-f8) | 4.74E+10 |

TABLE 23
(AUC data in FIG. 13B)

| Group | AUC (p/s*hour) |
|---|---|
| LQ104-E16b-2 (PEG-f9) | 1.61E+11 |
| LQ104-E16b-2 (PEG-f10) | 1.21E+12 |
| LQ104-E16b-2 (PEG-f11) | 9.72E+11 |
| LQ104-E16b-2 (PEG-f12) | 7.49E+11 |
| LQ104-E16b-2 (PEG-f13) | 4.30E+11 |
| LQ104-E16b-2 (PEG-f14) | 3.64E+11 |
| LQ104-E16b-2 (PEG-f15) | 1.71E+11 |
| LQ104-E16b-2 (PEG-f16) | 5.82E+10 |

In the related art, it is difficult to achieve a good delivery effect when the phospholipid content in the LNP preparation was below 4 mol %. However, in the LNP preparation prepared using the ionizable lipid compound provided by the present application, even when the phospholipid component was as low as 4 mol % or less, such as in a range from 0 mol % to 2 mol %, as demonstrated in this example, the prepared LNP preparation still had great properties and delivery ability. At the same time, without increasing the PEG lipid content, such as in the range of 0.5 mol % to 2.5 mol %, the prepared LNP preparation still had good properties and delivery ability, thereby reducing the risk of affecting efficacy and safety by PEG lipid increase.

What is claimed is:

1. A nitrogen-containing chain compound represented by a formula (I) or a pharmaceutically acceptable salt thereof, characterized in that, 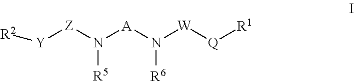
wherein, Z and W are independently C₃-C₁₀ alkylene;
Y and Q are independently; 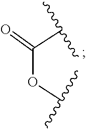 [SMILES: *OC(*)=O]
A is C₂-C₆ alkylene, 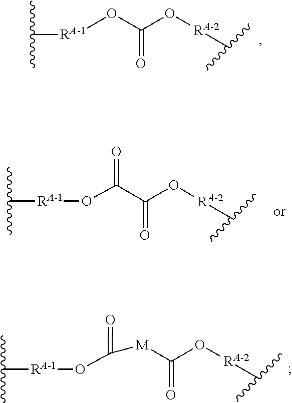
R^{A-1} and R^{A-2} are each independently C₂-C₆ alkylene;
M is C₁-C₆ alkylene;
R¹ and R² are independently C₆-C₂₀ alkyl;
R⁵ is C₂-C₁₀ alkyl that is unsubstituted or substituted with 1, 2, or 3 R⁵⁻¹;
each R⁵⁻¹ is independently hydroxyl or 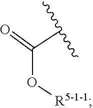
each R⁵⁻¹⁻¹ is independently C₆-C₂₀ alkyl;
R⁶ is C₂-C₁₀ alkyl that is unsubstituted or substituted with 1, 2, or 3 R⁶⁻¹;
each R⁶⁻¹ is independently hydroxyl or 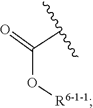
and
each R⁶⁻¹⁻¹ is independently C₆-C₂₀ alkyl.

2. The nitrogen-containing chain compound represented by the formula (I) or the pharmaceutically acceptable salt thereof of claim 1, characterized in that, 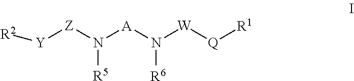
wherein, Z and W are independently C₄-C₁₀ alkylene;
Y and Q are independently 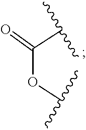 [SMILES: *OC(*)=O]
A is C₂-C₆ alkylene, 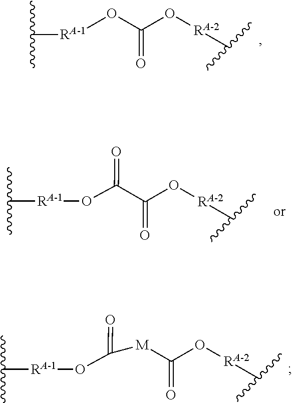
R^{A-1} and R^{A-2} are each independently C₂-C₆ alkylene;
M is C₁-C₆ alkylene;
R¹ and R² are independently C₆-C₂₀ alkyl;
R⁵ is C₂-C₁₀ alkyl that is unsubstituted or substituted with 1, 2, or 3 R⁵⁻¹;
each R⁵⁻¹ is independently hydroxyl or 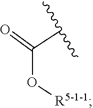
R⁵⁻¹⁻¹ is independently C₆-C₂₀ alkyl;
R⁶ is C₂-C₁₀ alkyl that is unsubstituted or substituted with 1, 2, or 3 R⁶⁻¹;
each R⁶⁻¹ is independently hydroxyl or 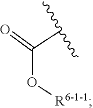
and
R⁶⁻¹⁻¹ is independently C₆-C₂₀ alkyl.

3. The nitrogen-containing chain compound represented by the formula (I) or the pharmaceutically acceptable salt thereof of claim 1, characterized in that the nitrogen-containing chain compound represented by the formula (I) meets one or more of the following conditions:
(1) in Z, C₃-C₁₀ alkylene is C₃-C₈ alkylene, preferably straight-chain alkane, such as 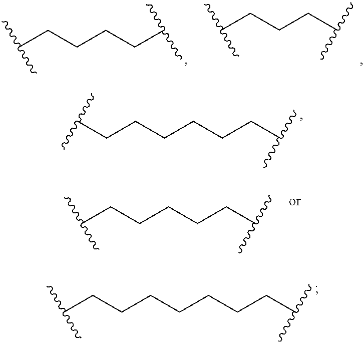 [SMILES: *CCC* ; *CCCC* ; *CCCCCC* ; *CCCCC* ; *CCCCCCC*]
(2) in W, C₃-C₁₀ alkylene is C₃-C₈ alkylene, preferably straight-chain alkane, such as 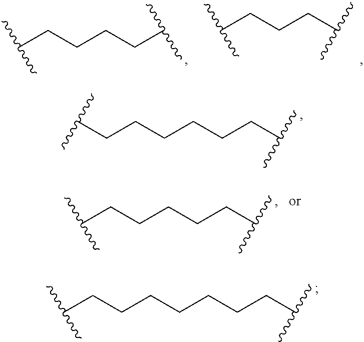 [SMILES: *CCC* ; *CCCC* ; *CCCCCC* ; *CCCCC* ; *CCCCCCC*]
(3) in A, C₂-C₆ alkylene is 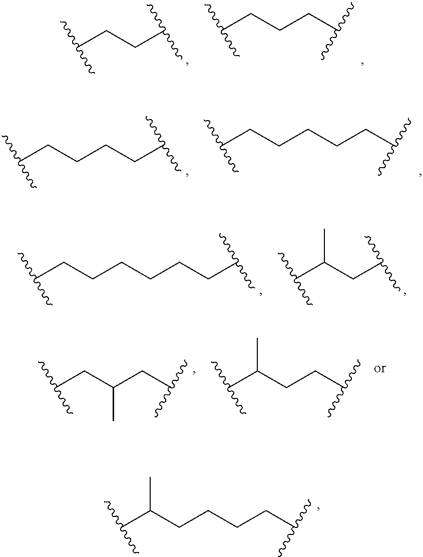
such as, 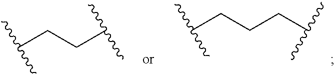
(4) in R¹, C₆-C₂₀ alkyl is C₁₀-C₁₉, such as 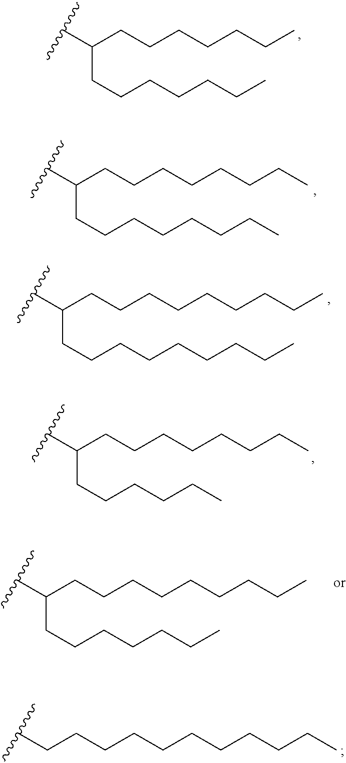 [SMILES: *C(CCCCCCC)CCCCCCC ; *C(CCCCCCCC)CCCCCCCC ; *C(CCCCCCCCC)CCCCCCCCC ; *C(CCCCCC)CCCCCCCC ; *C(CCCCCCC)CCCCCCCCC ; *CCCCCCCCCCC]
and
(5) in R², C₆-C₂₀ alkyl is C₁₀-C₁₉, such as

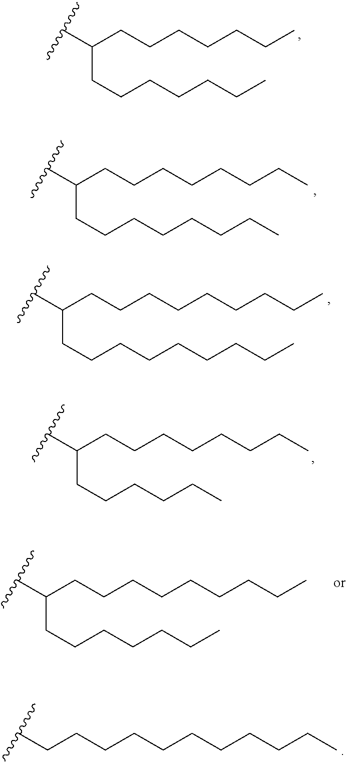 [SMILES: *C(CCCCCCC)CCCCCCC ; *C(CCCCCCCC)CCCCCCCC ; *C(CCCCCCCCC)CCCCCCCCC ; *C(CCCCCC)CCCCCCCC ; *C(CCCCCCC)CCCCCCCCC ; *CCCCCCCCCCC]

4. The nitrogen-containing chain compound represented by the formula (I) or the pharmaceutically acceptable salt thereof of claim 2, characterized in that the nitrogen-containing chain compound represented by the formula (I) meets one or more of the following conditions:
(1) in Z, C₄-C₁₀ alkylene is C₅-C₈ alkylene, preferably straight-chain alkane, such as 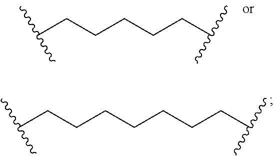 [SMILES: *CCCCC* ; *CCCCCCC*]
(2) in W, C₄-C₁₀ alkylene is C₅-C₈ alkylene, preferably straight-chain alkane, such as 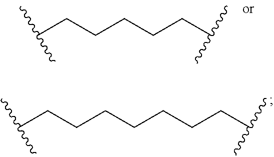 [SMILES: *CCCCC* ; *CCCCCCC*]
(3) in A C₂-C₆ alkylene is 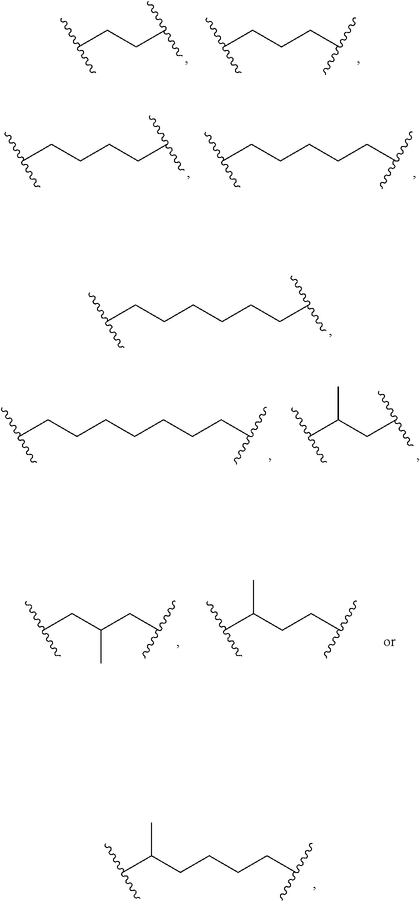
such as 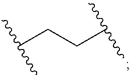 [SMILES: *CC*]
(4) in R^{A-1}, C₂-C₆ alkylene is 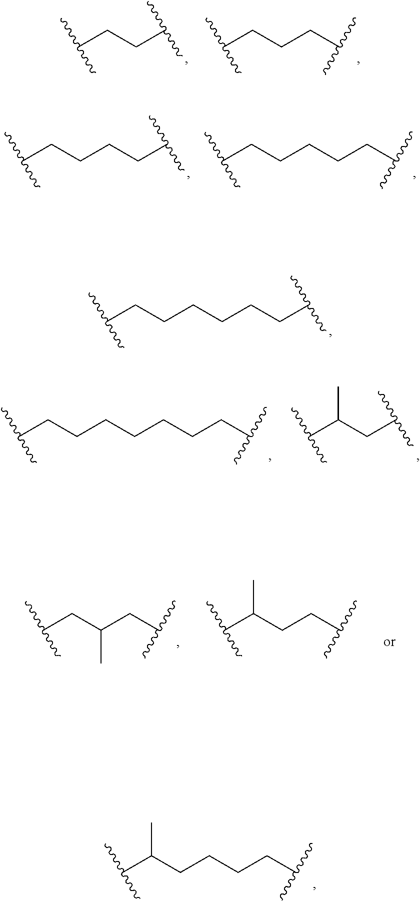
such as 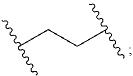 [SMILES: *CC*]
(5) in R^{A-2} C₂-C₆ alkylene is 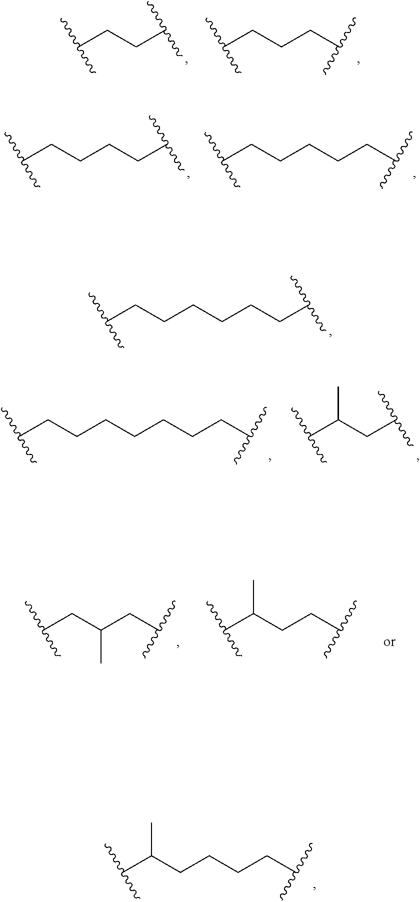
such as 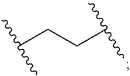 [SMILES: *CC*]
(6) in M, C₁-C₆ alkylene is 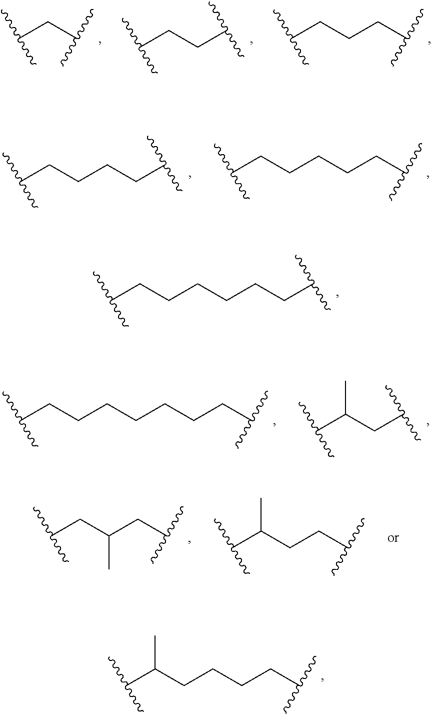
such as 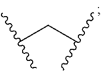 [SMILES: *C*]
(7) in R¹, C₆-C₂₀ alkyl is C₁₀-C₁₈, such as 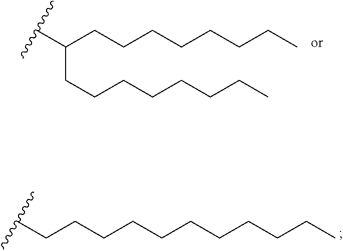 [SMILES: *C(CCCCCCCC)CCCCCCCC ; *CCCCCCCCCCC]
(8) in R², C₆-C₂₀ alkyl is C₁₀-C₁₈, such as 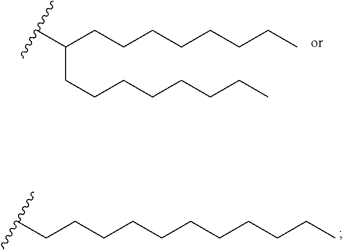 [SMILES: *C(CCCCCCCC)CCCCCCCC ; *CCCCCCCCCCC]
(9) in R⁵, C₂-C₁₀ alkyl is C₂-C₈ alkyl, such as 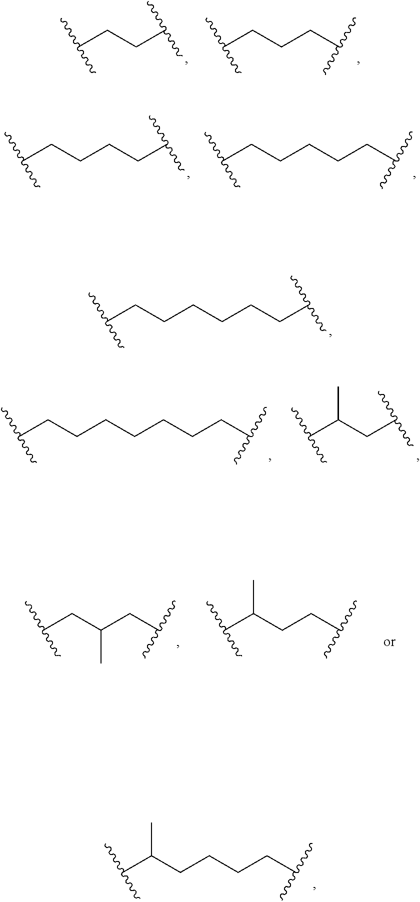
and also such as 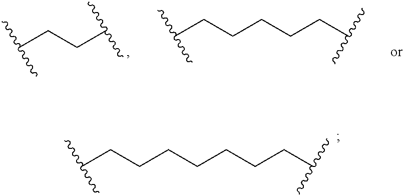
(10) in R⁵⁻¹⁻¹, C₆-C₂₀ alkyl is C₁₁-C₁₈, such as 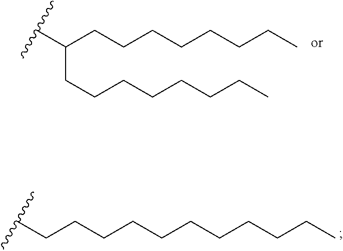 [SMILES: *C(CCCCCCCC)CCCCCCCC ; *CCCCCCCCCCC]
(11) in R⁶, C₂-C₁₀ alkyl is C₂-C₈ alkyl, such as 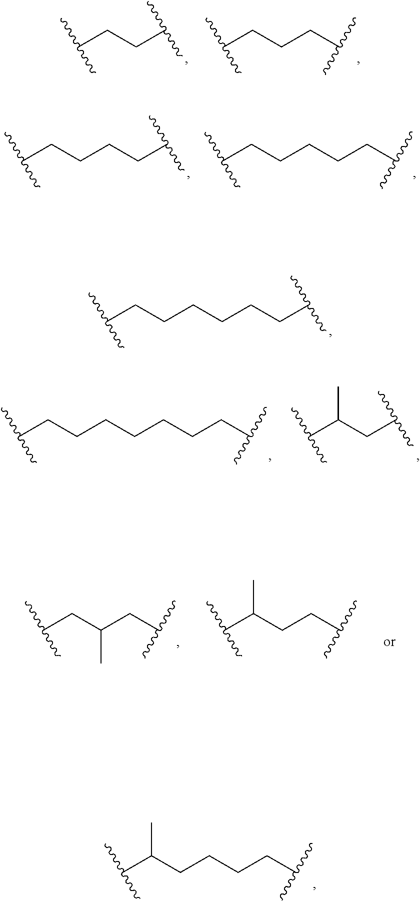
and also such as 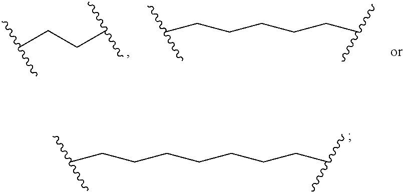
(12) in R⁶⁻¹⁻¹, C₆-C₂₀ alkyl is C₁₁-C₁₈, such as 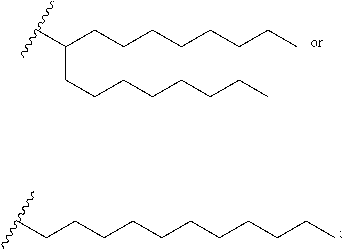 [SMILES: *C(CCCCCCCC)CCCCCCCC ; *CCCCCCCCCCC]
and
(13) the nitrogen-containing chain compound represented by the formula (I) is a nitrogen-containing chain compound represented by a formula (I-a)

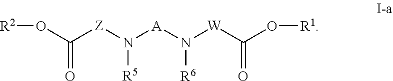

5. The nitrogen-containing chain compound represented by the formula (I) or the pharmaceutically acceptable salt thereof of claim 1, characterized in that the nitrogen-containing chain compound represented by the formula (I) meets one or more of the following conditions:
(1) Z and W are the same;
(2) R¹ and R² are the same;
(3) R⁵ and R⁶ are the same; and
(4) Q and Y are the same.

6. The nitrogen-containing chain compound represented by the formula (I) or the pharmaceutically acceptable salt thereof of claim 2, characterized in that the nitrogen-containing chain compound represented by the formula (I) meets one or more of the following conditions:
(1) Z and W are independently C₅-C₈ alkylene;
(2) A is C₂-C₆ alkylene, or 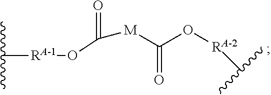
(3) R^{A-1} and R^{A-2} are independently C₂-C₄ alkylene;
(4) M is methylene;
(5) R¹ and R² are independently C₁₀-C₁₈, such as C₁₀-C₁₂, and also such as 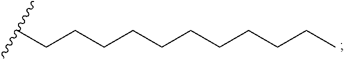 [SMILES: *CCCCCCCCCCC]
(6) R⁵ is C₂-C₈ alkyl that is substituted with 1, 2, or 3 R⁵⁻¹;
(7) R⁵⁻¹⁻¹ is C₁₀-C₁₈ alkyl, such as C₁₄-C₁₈ alkyl, and also such as 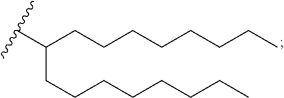 [SMILES: *C(CCCCCCCC)CCCCCCCC]
(8) R⁶ is C₂-C₈ alkyl that is substituted with 1, 2, or 3 R⁶⁻¹;
(9) R⁶⁻¹⁻¹ is C₁₀-C₁₈ alkyl, such as C₁₄-C₁₈ alkyl, and also such as 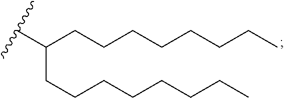 [SMILES: *C(CCCCCCCC)CCCCCCCC]
(10) the nitrogen-containing chain compound represented by the formula (I) is a bilaterally symmetrical compound;
(11) Y is 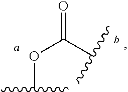
wherein a is connected to R², and b is connected to Z; and
(12) Q is 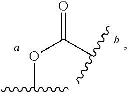
wherein a is connected to R¹, and b is connected to W;
preferably, the nitrogen-containing chain compound represented by the formula (I) is a solution 1 or a solution 2:
solution 1:
Y is 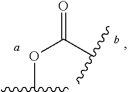
wherein a is connected to R², and b is connected to Z;
Q is 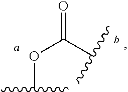
wherein a is connected to R¹, and b is connected to W;
Z and W are independently C₅-C₈ alkylene;
A is C₂-C₆ alkylene, or 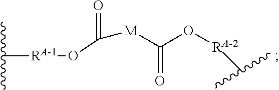
R^{A-1} and R^{A-2} are independently C₂-C₄ alkylene;
M is methylene;
R¹ and R² are independently C₁₀-C₁₈;
R⁵ is C₂-C₈ alkyl that is substituted with 1, 2, or 3 R⁵⁻¹;
R⁵⁻¹⁻¹ is C₁₀-C₁₈ alkyl;
R⁶ is C₂-C₈ alkyl that is substituted with 1, 2, or 3 R⁶⁻¹; and
R⁶⁻¹⁻¹ is C₁₀-C₁₈ alkyl;
solution 2:
Q and Y are the same;
Z and W are the same;
R¹ and R² are the same;
R⁵ and R⁶ are the same;
Z and W are independently C₅-C₈ alkylene;
A is C₂-C₆ alkylene, or 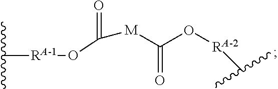
R^{A-1} and R^{A-2} are independently C₂-C₄ alkylene;
M is methylene;
R¹ and R² are independently 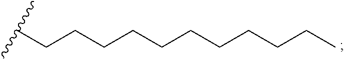 [SMILES: *CCCCCCCCCCC]
R⁵ is C₂-C₈ alkyl that is substituted with 1, 2, or 3 R⁵⁻¹;
R⁵⁻¹⁻¹ is C₁₄-C₁₈ alkyl;
R⁶ is C₂-C₈ alkyl that is substituted with 1, 2, or 3 R⁶⁻¹; and
R⁶⁻¹⁻¹ is C₁₄-C₁₈ alkyl.

7. The nitrogen-containing chain compound represented by the formula (I) or the pharmaceutically acceptable salt thereof of claim 1, characterized in that the nitrogen-containing chain compound represented by the formula (I) meets one or more of the following conditions:
(1) Z and W are independently C₃-C₈ alkylene;
(2) A is C₂-C₆ alkylene, or 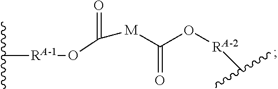
(3) R^{A-1} and R^{A-2} are independently C₂-C₄ alkylene;
(4) M is methylene;
(5) R¹ and R² are independently C₁₀-C₂₀ alkyl preferably 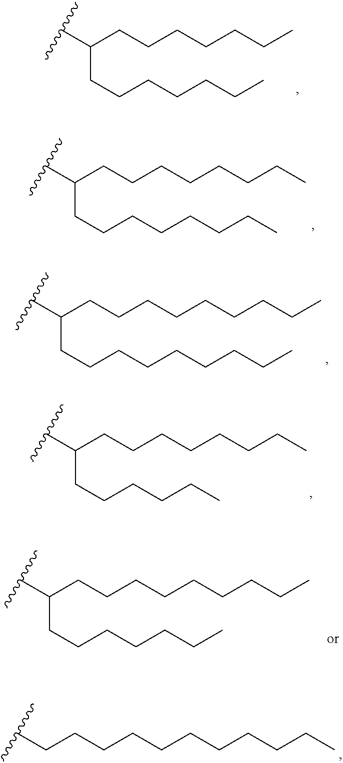 [SMILES: *C(CCCCCCC)CCCCCCC ; *C(CCCCCCCC)CCCCCCCC ; *C(CCCCCCCCC)CCCCCCCCC ; *C(CCCCCC)CCCCCCCC ; *C(CCCCCCC)CCCCCCCCC ; *CCCCCCCCCCC]
and more preferably 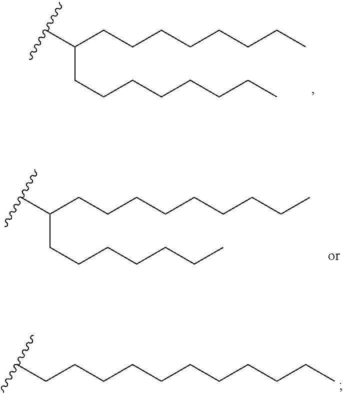 [SMILES: *C(CCCCCCCC)CCCCCCCC ; *C(CCCCCCC)CCCCCCCCC ; *CCCCCCCCCCC]
(6) R⁵ is C₂-C₈ alkyl that is substituted with 1, 2, or 3 R⁵⁻¹;
(7) R⁵⁻¹⁻¹ is C₁₀-C₁₈ alkyl, such as C₁₄-C₁₈ alkyl, and also such as 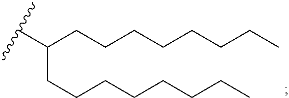 [SMILES: *C(CCCCCCCC)CCCCCCCC]
(8) R⁶ is C₂-C₈ alkyl that is substituted with 1, 2, or 3 R⁶⁻¹;
(9) R⁶⁻¹⁻¹ is C₁₀-C₁₈ alkyl, such as C₁₄-C₁₈ alkyl, and also such as 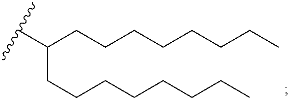 [SMILES: *C(CCCCCCCC)CCCCCCCC]
(10) the nitrogen-containing chain compound represented by the formula (I) is a bilaterally symmetrical compound;
(11) Y is 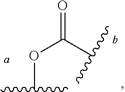
wherein a is connected to R², and b is connected to Z; and
(12) Q is 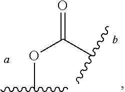
wherein a is connected to R¹, and b is connected to W;
preferably, the nitrogen-containing chain compound represented by the formula (I) is a solution 3:
Y is 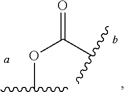
wherein a is connected to R², and b is connected to Z;
Q is 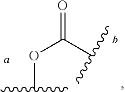
wherein a is connected to R¹, and b is connected to W;
Z and W are independently C₃-C₈ alkylene;
A is C₂-C₆ alkylene, or 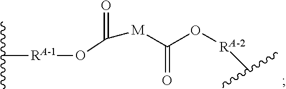
R^{A-1} and R^{A-2} are independently C₂-C₄ alkylene;
M is methylene;
R¹ and R² are independently C₁₀-C₂₀ alkyl;
R⁵ is C₂-C₈ alkyl that is substituted with 1, 2, or 3 R⁵⁻¹; and
R⁵⁻¹⁻¹ is C₁₀-C₁₈ alkyl;
R⁶ is C₂-C₈ alkyl that is substituted with 1, 2, or 3 R⁶⁻¹; and
R⁶⁻¹⁻¹ is C₁₀-C₁₈ alkyl.

8. The nitrogen-containing chain compound represented by the formula (I) or the pharmaceutically acceptable salt thereof of claim 1, characterized in that the nitrogen-containing chain compound represented by the formula (I) meets one or more of the following conditions:
(1) Z is 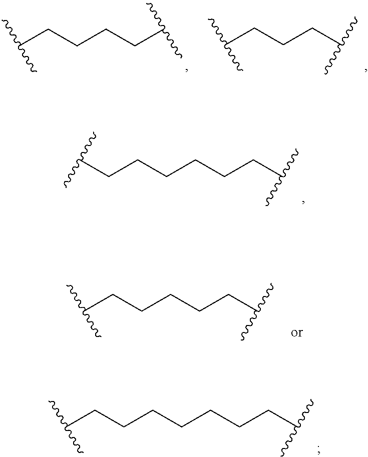 [SMILES: *CCCC* ; *CCC* ; *CCCCCC* ; *CCCCC* ; *CCCCCCC*]
(2) W is 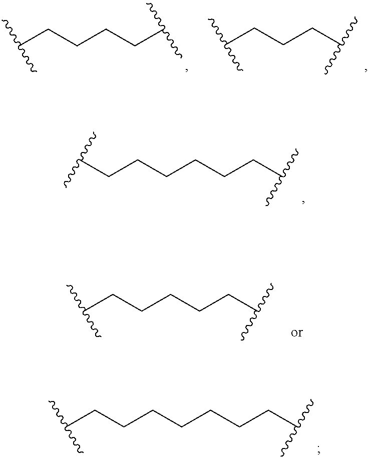 [SMILES: *CCCC* ; *CCC* ; *CCCCCC* ; *CCCCC* ; *CCCCCCC*]
(3) R¹ is 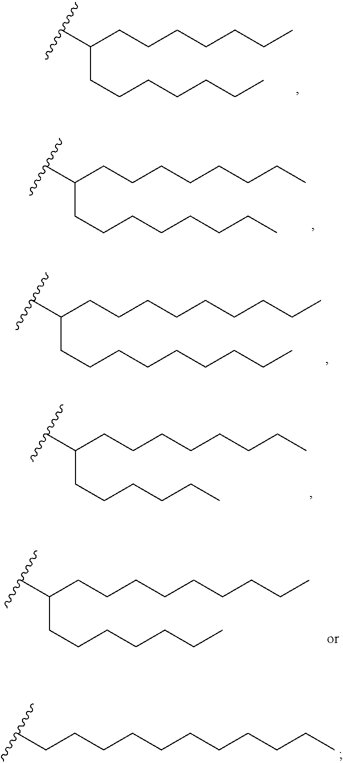 [SMILES: *C(CCCCCCC)CCCCCCC ; *C(CCCCCCCC)CCCCCCCC ; *C(CCCCCCCCC)CCCCCCCCC ; *C(CCCCCC)CCCCCCCC ; *C(CCCCCCC)CCCCCCCCC ; *CCCCCCCCCCC]
(4) R² is 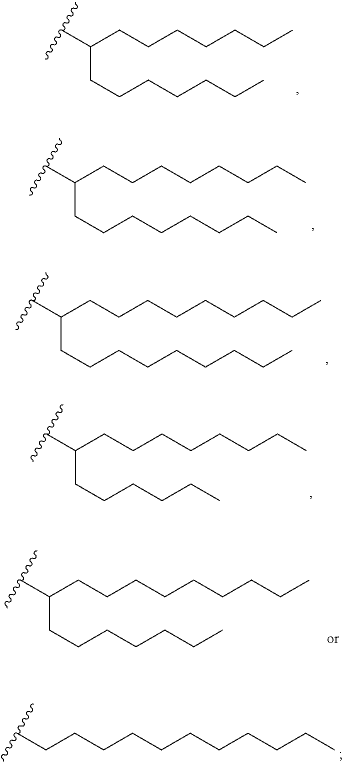 [SMILES: *C(CCCCCCC)CCCCCCC ; *C(CCCCCCCC)CCCCCCCC ; *C(CCCCCCCCC)CCCCCCCCC ; *C(CCCCCC)CCCCCCCC ; *C(CCCCCCC)CCCCCCCCC ; *CCCCCCCCCCC]
(5) R⁵ is 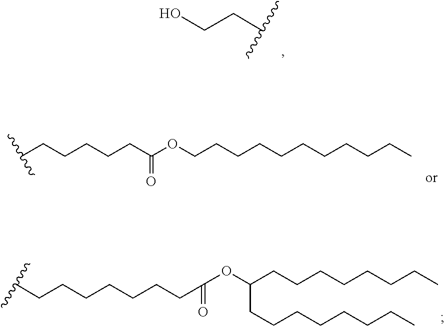
(6) R⁶ is 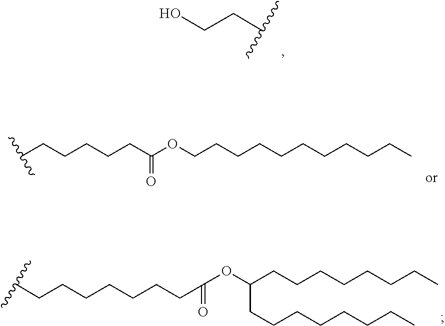
and
(7) A is

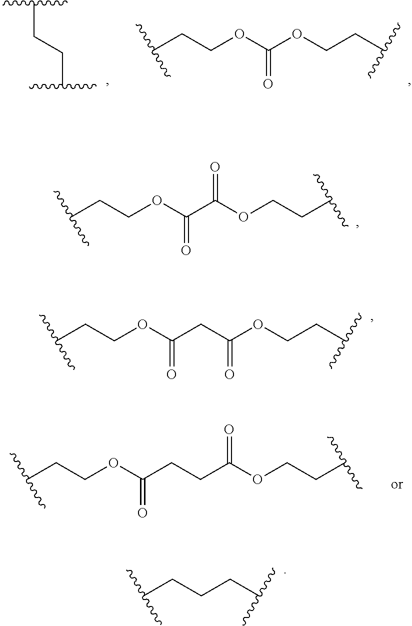

9. The nitrogen-containing chain compound represented by the formula (I) or the pharmaceutically acceptable salt thereof of claim 1, characterized in that the nitrogen-containing chain compound represented by the formula (I) is any one of the following compounds:

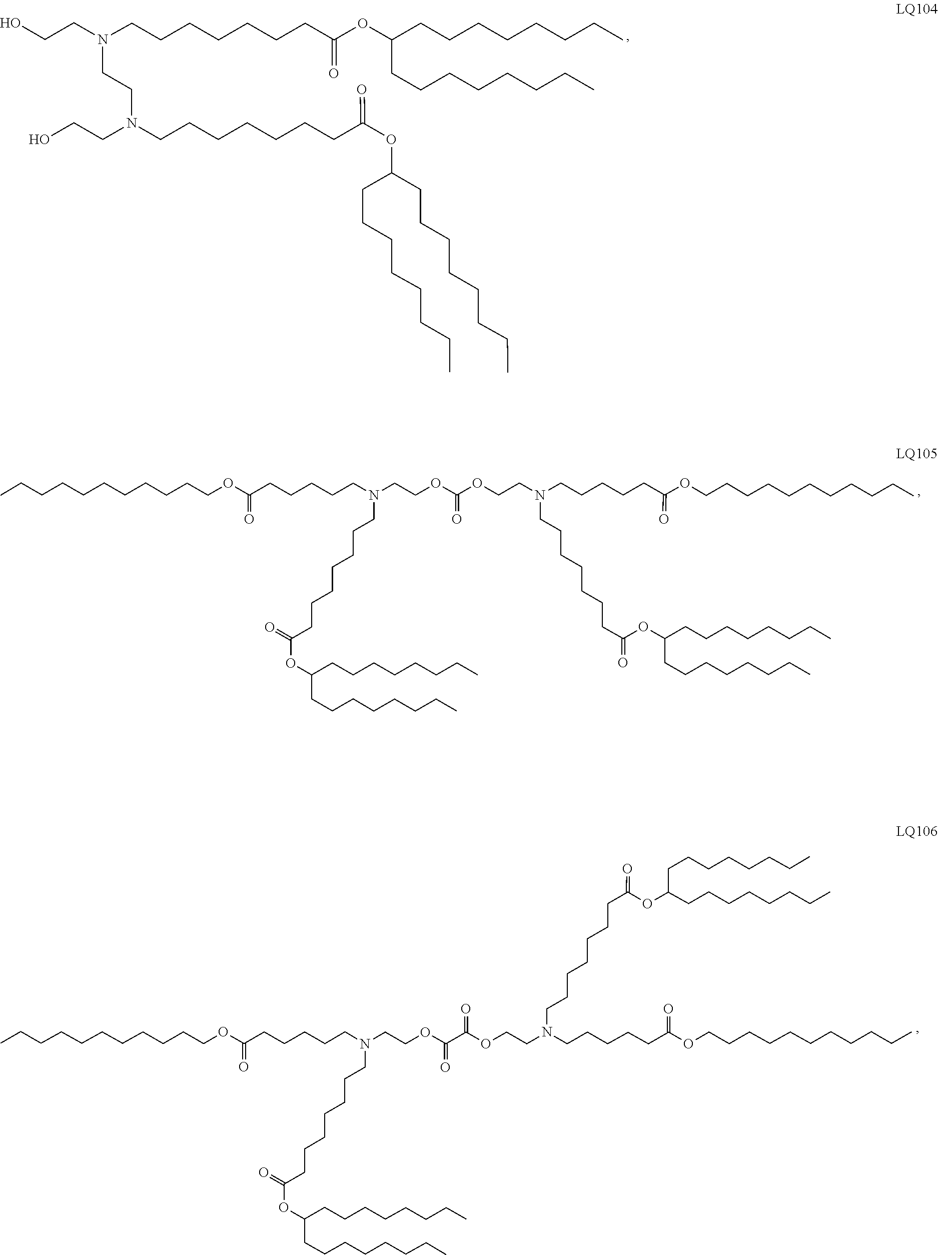

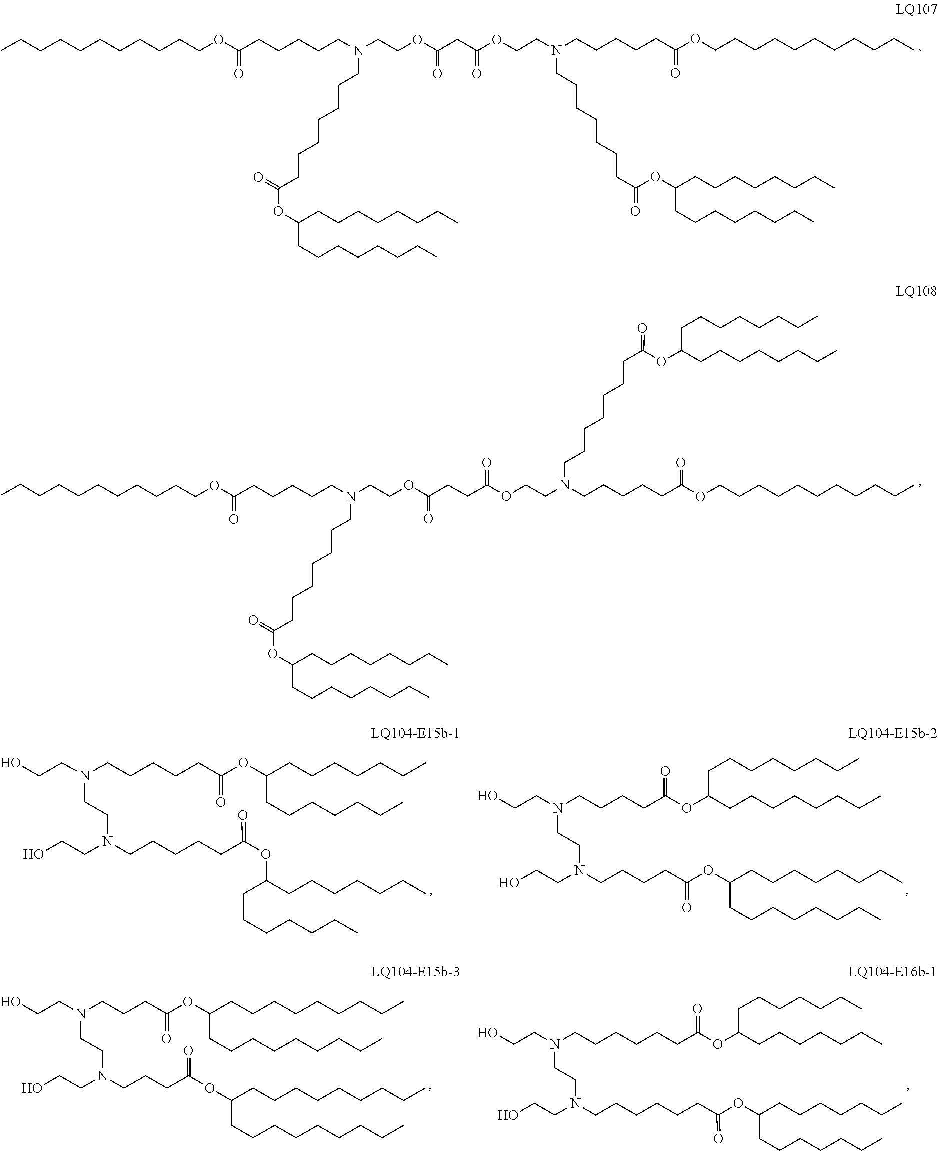

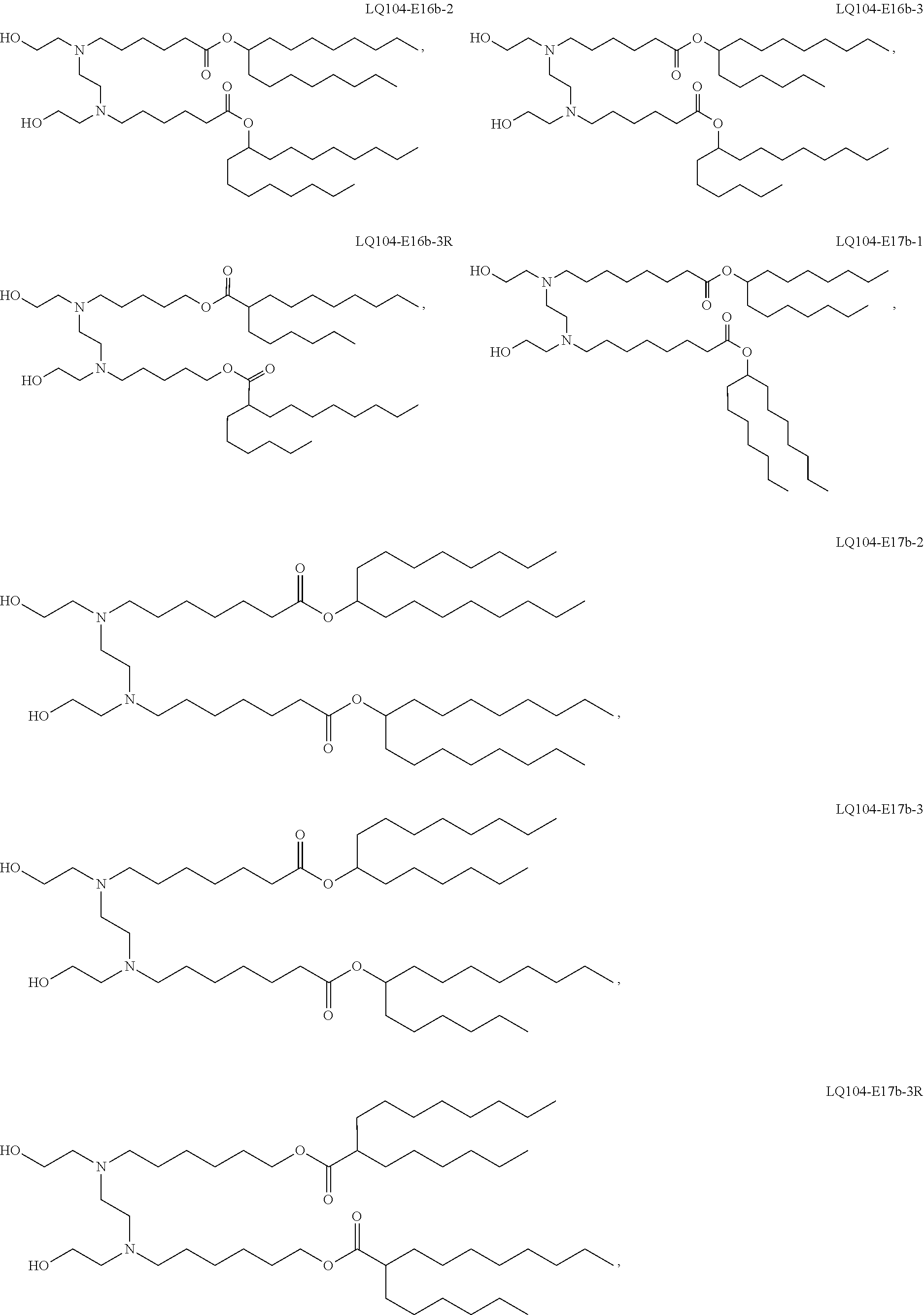

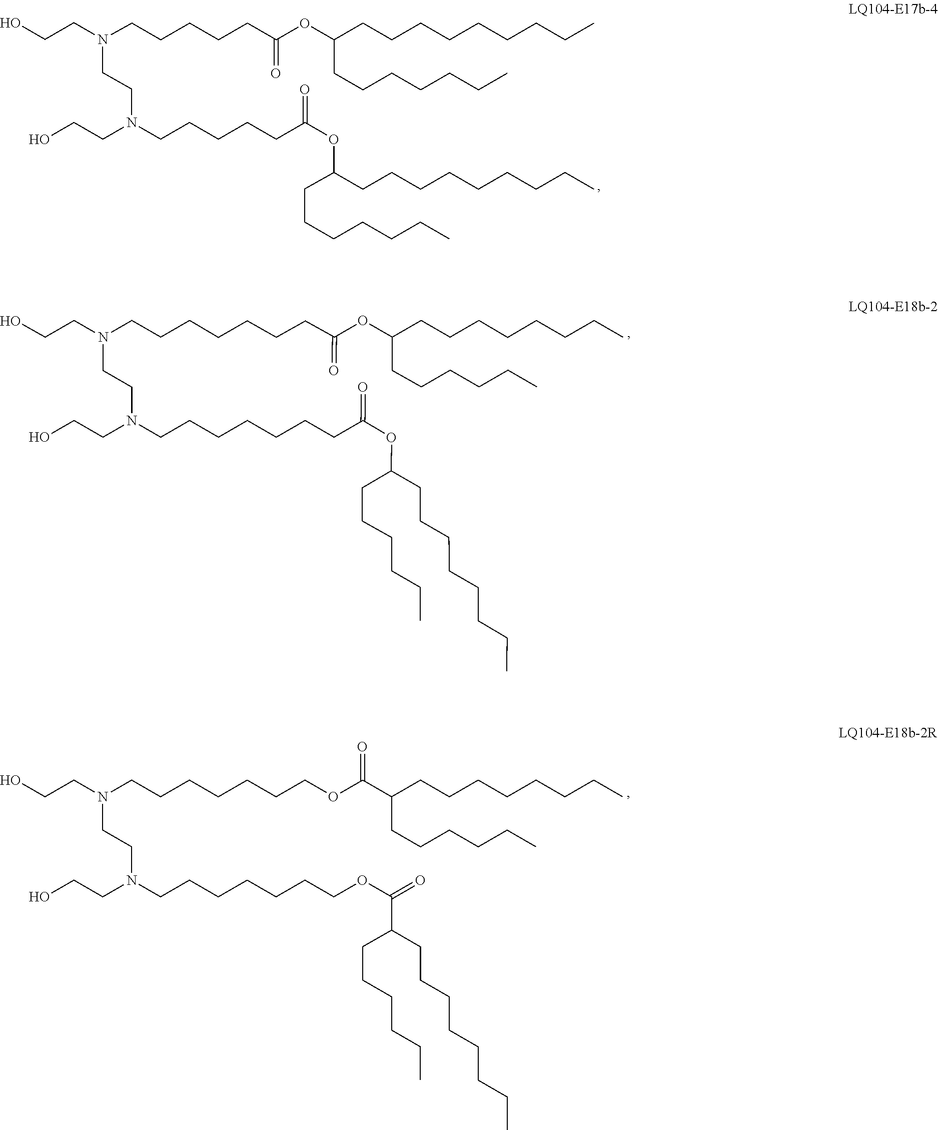 [SMILES: CCCCCCCCCC(CCCCCCC)OC(=O)CCCCCN(CCO)CCN(CCO)CCCCCC(=O)OC(CCCCCCC)CCCCCCCCC ; CCCCCCCCC(CCCCCC)OC(=O)CCCCCCCN(CCO)CCN(CCO)CCCCCCCC(=O)OC(CCCCCC)CCCCCCCC ; CCCCCCCCC(CCCCCC)C(=O)OCCCCCCCN(CCO)CCN(CCO)CCCCCCCOC(=O)C(CCCCCC)CCCCCCCC]

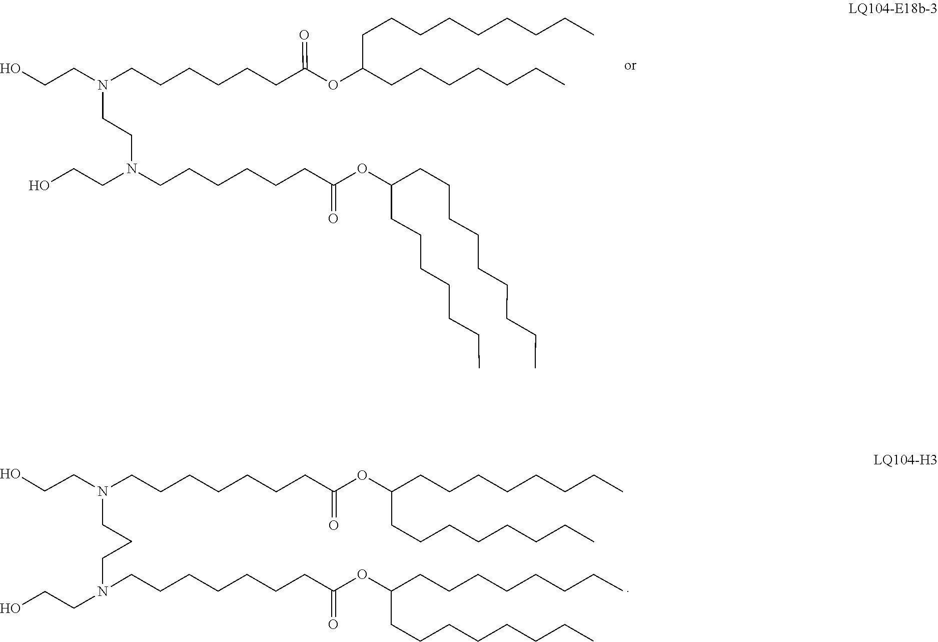 [SMILES: CCCCCCCCCC(CCCCCCC)OC(=O)CCCCCCN(CCO)CCN(CCO)CCCCCCC(=O)OC(CCCCCCC)CCCCCCCCC ; CCCCCCCCC(CCCCCCCC)OC(=O)CCCCCCCN(CCO)CCCN(CCO)CCCCCCCC(=O)OC(CCCCCCCC)CCCCCCCC]

10. A preparation method of the nitrogen-containing chain compound represented by the formula (I), characterized by comprising the following steps: performing, in a solvent and in the presence of a base and an iodized salt, a coupling reaction represented by the following formula on a compound represented by a formula (I-1) and a compound represented by a formula (I-2); 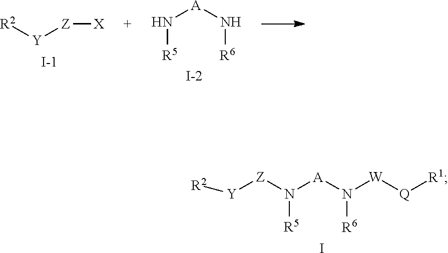
wherein, X is halogen, A is ₂-C₆ alkylene, and Y, Q, Z, W, R⁵, R⁶, R¹ and R² are described as claim 1; and Y is the same as Q, R¹ is the same as R², and Z is the same as W;
preferably, the preparation method of the nitrogen-containing chain compound represented by the formula (I) meets one or more of the following conditions:
(1) in the coupling reaction, the halogen is fluorine, chlorine, bromine, or iodine, such as bromine;
(2) in the coupling reaction, a molar ratio of the compound represented by the formula (I-2) to the compound represented by the formula (I-1) is 1:(1-3), such as 1:2.6;
(3) in the coupling reaction, the base is basic carbonate, such as K₂CO₃;
(4) in the coupling reaction, a molar ratio of the compound represented by the formula (I-2) to the base is 1:(1-5), such as 1:3.5;
(5) in the coupling reaction, the solvent is an ether solvent or/and a nitrile solvent; the ether solvent may be methyl tert-butyl ether; the nitrile solvent may be acetonitrile; and a volume ratio of the nitrile solvent to the ether solvent may be 1:1;
(6) in the coupling reaction, a mass-to-volume ratio of the compound represented by the formula (I-2) to the solvent is in a range from 10 mg/mL to 50 mg/mL, such as 16 mg/mL;
(7) in the coupling reaction, the iodized salt is a basic iodized salt, such as KI;
(8) in the coupling reaction, a molar ratio of the compound represented by the formula (I-2) to the iodized salt is 1:(1-2), such as 1:1.2; and
(9) in the coupling reaction, a reaction temperature of the coupling reaction is in a range from 50° C. to 100° C., such as 80° C.

11. A lipid carrier, characterized by comprising a substance Z, wherein the substance Z is selected from one or more of the compound represented by the formula (I) of claim 1 or a pharmaceutically acceptable salt thereof.

12. The lipid carrier of claim 11, characterized in that the lipid carrier meets one or more of the following conditions:
(1) the lipid carrier further comprises a diluent;
(2) the lipid carrier further comprises phospholipid;
(3) the lipid carrier further comprises PEG lipid; and
(4) the lipid carrier further comprises sterol.

13. The lipid carrier of claim 12, characterized in that the lipid carrier meets one or more of the following conditions:
(1) the diluent is phosphate buffer saline or Tris buffer;
(2) the phospholipid is a phospholipid molecule with a charged polar end and a fatty chain non-polar end, such as distearoyl phosphatidylcholine, 1,2-dimyristoyl-sn-glycero-3-phosphocholine, dioleoyl phosphorylcholine, palmitoyl phosphorylcholine, 1,2-distearoyl phosphatidylcholine, nonadecanoyl phosphatidylcholine or palmitoyl phosphorylcholine;
(3) the PEG lipid is a lipid molecule modified with a polyethylene glycol hydrophilic end; preferably, the PEG lipid is preferably selected from one or more of PEG-modified phosphatidyl ethanolamine, PEG-modified phosphatidic acid, PEG-modified ceramide, PEG-modified dialkyl amine, PEG-modified diacylglycerol, and PEG-modified dialkylglycerol, for example, the PEG lipid is PEG-modified dimyristoyl glycerol;
(4) the sterol comprises animal, plant, or fungal sterols; and preferably, the sterol is selected from one or more of cholesterol, sitosterol, ergosterol, campesterol, stigmasterol, brassinosteroid, tomatidine, ursolic acid, and α-tocopherol, such as cholesterol;
(5) in the lipid carrier, a molar ratio of the substance Z to the sterol is (0.5-5):1, preferably (0.5-3):1, such as (0.6-2):1; and also such as, 0.66:1, 0.68:1, 0.69:1, 0.70:1, 0.71:1, 0.72:1, 0.74:1, 0.76:1, 0.77:1, 0.79:1, 0.82:1, 0.83:1, 0.84:1, 0.85:1, 0.86:1, 0.87:1, 0.88:1, 0.89:1, 0.9:1, 0.91:1, 0.92:1, 0.93:1, 0.94:1, 0.97:1, 0.99:1, 1.04:1, 1.07:1, 1.1:1, 1.16:1, 1.23:1, 1.28:1, 1.30:1, 1.32:1, 1.41:1, 1.52:1, 1.58:1, 1.64:1, 1.65:1, 1.74:1, 1.79:1 or 1.96:1;
(6) in the lipid carrier, a molar ratio of the substance Z to the phospholipid is (1-25):1, preferably (2-25):1, such as 22.5:1, 20:1, 17.5:1, 15:1, 11.25:1, 10:1, 8.75:1, 7.5:1, 6.67:1, 5:1, 4.75:1, 4.5:1, 4:1, 3.9:1, 3.6:1, 3.3:1, 3:1, 2.86:1, 2.5:1 or 2.2:1;
(7) in the lipid carrier, a molar ratio of the substance Z to the PEG lipid is (16-130):1, preferably (16-80):1, such as (16-40):1; and also such as 16:1, 18:1, 18.8:1, 20:1, 21.9:1, 22.5:1, 25:1, 26.9:1, 27.5:1, 28.1:1, 29.6:1, 30:1, 31.25:1, 33.3:1, or 37.5:1;
(8) a molar content of the substance Z is in a range from 30 mol % to 60 mol %; preferably in a range from 40 mol % to 55 mol %, such as 40 mol %, 43 mol %, 45 mol %, 47.4 mol %, 50 mol %, or 55 mol %;
(9) a molar content of the phospholipid is in a range from 0 mol % to 30 mol %; preferably in a range from 0 mol % to 18 mol %, such as 0 mol %, 2 mol %, 4 mol %, 6 mol %, 8 mol %, 10 mol %, 11 mol %, 12 mol %, 14 mol %, 16 mol %, or 18 mol %;
(10) a molar content of the sterol is in a range from 15 mol % to 60 mol %; preferably in a range from 40.4 mol % to 58.4 mol %, such as 40.4 mol %, 41 mol %, 42.4 mol %, 43 mol %, 43.4 mol %, 44.4 mol %, 46.4 mol %, 47.4 mol %, 48 mol %, 48.4 mol %, 49 mol %, 49.4 mol %, 49.5 mol %, 50 mol %, 50.4 mol %, 50.5 mol %, 51 mol %, 51.4 mol %, 51.5 mol %, 52 mol %, 52.25 mol %, 52.4 mol %, 52.5 mol %, 52.75 mol %, 53 mol %, 53.4 mol %, 54 mol %, 54.25 mol %, 54.4 mol %, 54.5 mol %, 54.75 mol %, 55 mol %, 56 mol %, 56.4 mol %, 56.5 mol %, 57 mol %, 57.5 mol %, 58 mol % or 58.4 mol %; and
(11) a molar content of the PEG lipid is in a range from 0 mol % to 10 mol %; such as in a range from 0.5 mol % to 2.5 mol %, and also such as 0.25 mol %, 0.5 mol %, 0.75 mol %, 1 mol %, 1.5 mol %, 1.6 mol %, 2 mol %, 2.5 mol %, 3 mol %, 3.5 mol %, 4 mol %, or 5 mol %; further in a range from 0.5 mol % to 2 mol %; it may be in a range from 0.5 mol % to 1.5 mol %, or in a range from 1.5 mol % to 2.5 mol %; and it may further be in a range from 1.6 mol % or 2 mol %;
preferably, the lipid carrier is a solution 1, a solution 2, a solution 3 or a solution 4:
solution 1: the lipid carrier consists of the substance Z, the diluent, the phospholipid, the PEG lipid and the sterol;
solution 2: the lipid carrier consists of the substance Z, the diluent, the PEG lipid and the sterol;
solution 3: the lipid carrier consists of the substance Z, the phospholipid, the PEG lipid and the sterol; and
solution 4: the lipid carrier consists of the substance Z, the PEG lipid and the sterol.

14. The lipid carrier of claim 11, characterized in that the lipid carrier does not contain phospholipid;
preferably, the lipid carrier meets one or more of the following conditions:
(1) when the lipid carrier does not contain the phospholipid, in the lipid carrier, a molar ratio of a substance Z to sterol is (0.6-2):1; preferably 0.68:1, 0.69:1, 0.70:1, 0.71:1, 0.72:1, 0.74:1, 0.76:1, 0.77:1, 0.79:1, 0.82:1, 0.83:1, 0.84:1, 0.85:1, 0.86:1, 0.87:1, 0.88:1, 0.89:1, 0.9:1, 0.91:1, 0.92:1, 0.93:1, 0.94:1, 0.97:1, 0.99:1, 1.04:1, 1.07:1, 1.1:1, 1.16:1, 1.23:1, 1.28:1, 1.30:1, 1.41:1, 1.52:1 or 1.58:1;
(2) when the lipid carrier does not contain the phospholipid, in the lipid carrier, a molar ratio of the substance Z to PEG lipid is (16-35):1, preferably 16:1, 18:1, 20:1, 21.9:1, 22.5:1, 25:1, 26.9:1, 27.5:1, 28.1:1, 29.6:1 or 30:1;
(3) when the lipid carrier does not contain the phospholipid, a molar content of the sterol in the lipid carrier is about in a range from 15 mol % to 60 mol %, preferably in a range from 40.4% mol % to 58.4 mol %, such as 43 mol %, 43.4 mol %, 44.4 mol %, 45 mol %, 46.4 mol %, 47.4 mol %, 48 mol %, 48.4 mol %, 49 mol %, 49.4 mol %, 49.5 mol %, 50 mol %, 50.4 mol %, 50.5 mol %, 51 mol %, 51.4 mol %, 51.5 mol %, 52 mol %, 52.4 mol %, 52.25 mol %, 52.5 mol %, 52.75 mol %, 53 mol %, 53.4 mol %, 54 mol %, 54.2 mol %, 53.4 mol %, 54 mol %, 54.25 mol %, 54.4 mol %, 54.5 mol %, 54.75 mol %, 55 mol %, 56 mol %, 56.4 mol %, 56.5 mol %, 57 mol %, 57.5 mol %, 58 mol % or 58.4 mol %; and also such as in a range from 52.5 mol % to 54.5 mol %, and further such as in a range from 53 mol % to 54.5 mol %; and
(4) when the lipid carrier does not contain the phospholipid, or when a content of the phospholipid is below 4 mol %, a molar content of the PEG lipid in the lipid carrier is about in a range from 0 mol % to 10 mol %, such as 0.25 mol %, 0.5 mol %, 0.75 mol %, 1 mol %, 1.5 mol %, 1.6 mol %, 2 mol %, 2.5 mol %, 3 mol %, 3.5 mol %, 4 mol % or 5 mol %; preferably, in a range from 0.25 mol % to 3 mol %, such as in a range from 0.5 mol % to 2.5 mol %, or in a range from 0.5 mol % to 2 mol %.

15. A lipid nanoparticle, characterized by comprising a therapeutic agent or a prophylactic agent and the lipid carrier of claim 11.

16. The lipid nanoparticle of claim 15, characterized in that the lipid nanoparticle meets one or more of the following conditions:
(1) the therapeutic agent or the prophylactic agent are/is one or two or more nucleic acids; preferably, the therapeutic agent or the prophylactic agent are/is single-stranded deoxyribonucleic acid, double-stranded DNA, small interfering RNA, asymmetric double-stranded small interfering RNA, microRNA, small hairpin RNA, circular RNA, transfer RNA or messenger RNA, preferably mRNA; such as firefly luciferase mRNA or SARS-CoV-2 spike protein mRNA;
(2) a nitrogen-to-phosphorus ratio in the lipid nanoparticle is in a range from 2:1 to 30:1, preferably in a range from 2:1 to 20:1, such as in a range from 3:1 to 20:1, and also such as in a range from 3:1 to 16:1;
(3) a particle size of the lipid nanoparticle is in a range from 10 nm to 200 nm, preferably in a range from 40 nm to 150 nm, such as in a range from 60 nm to 150 nm, and also such as in a range from 50 nm to 150 nm;
(4) in the lipid nanoparticle, a mass ratio of the lipid carrier to the therapeutic agent or the prophylactic agent is in a range from (3-80):1, preferably in a range from (6-60):1; and
(5) in the lipid nanoparticle, the lipid carrier encapsulates the therapeutic agent or the prophylactic agent.

17. A composition, characterized by comprising a substance Z, wherein the substance Z is the compound represented by the formula (I) or the pharmaceutically acceptable salt thereof of claim 1.

18. The composition of claim 17, characterized by further comprising one or more of a diluent, phospholipid, PEG lipid, sterol, and a therapeutic agent or a prophylactic agent; wherein
preferably, in the composition, the diluent is phosphate buffer saline or Tris buffer, the phospholipid is a phospholipid molecule with a charged polar end and a fatty chain non-polar end, such as distearoyl phosphatidylcholine, 1,2-dimyristoyl-sn-glycero-3-phosphocholine, dioleoyl phosphorylcholine, palmitoyl phosphorylcholine, 1,2-distearoyl phosphatidylcholine, nonadecanoyl phosphatidylcholine or palmitoyl phosphorylcholine, the PEG lipid is a lipid molecule modified with a polyethylene glycol hydrophilic end; preferably, the PEG lipid is preferably selected from one or more of PEG-modified phosphatidyl ethanolamine, PEG-modified phosphatidic acid, PEG-modified ceramide, PEG-modified dialkyl amine, PEG-modified diacylglycerol, and PEG-modified dialkylglycerol, for example, the PEG lipid is PEG-modified dimyristoyl glycerol, and the sterol comprises animal, plant, or fungal sterols; and preferably, the sterol is selected from one or more of cholesterol, sitosterol, ergosterol, campesterol, stigmasterol, brassinosteroid, tomatidine, ursolic acid, and α-tocopherol, such as cholesterol; or, the therapeutic agent or the prophylactic agent are/is one or two or more nucleic acids; preferably, the therapeutic agent or the prophylactic agent are/is single-stranded deoxyribonucleic acid, double-stranded DNA, small interfering RNA, asymmetric double-stranded small interfering RNA, microRNA, small hairpin RNA, circular RNA, transfer RNA or messenger RNA, preferably mRNA; such as firefly luciferase mRNA or SARS-CoV-2 spike protein mRNA;
more preferably, in the composition, the substance Z forms the lipid carrier of claim 11 with one or more of the diluent, the phospholipid, the PEG lipid, and the sterol; or, in the composition, an encapsulation efficiency of the therapeutic agent or the prophylactic agent is at least 50%, preferably at least 70%;
further preferably, in the composition, the lipid carrier forms the lipid nanoparticle of claim 15 with the therapeutic agent or the prophylactic agent; or, in the composition, a polymer dispersity index of the composition is not higher than 0.5, such as not higher than 0.3.
